(19) United States
(12) Patent Application Publication
(10) Pub. No.: US 2024/0279666 A1
(43) Pub. Date: Aug. 22, 2024

(54) METHODS OF TREATING ALZHEIMER'S DISEASE

(71) Applicant: Aarhus Universitet, Aarhus C (DK)

(72) Inventor: Olav Michael Andersen, Aarhus C (DK)

(21) Appl. No.: 18/570,220

(22) PCT Filed: Jul. 1, 2022
(86) PCT No.: PCT/EP2022/068313

§ 371 Date: Dec. 14, 2023

(30) Foreign Application Priority Data
Jul. 2, 2021 (EP) 21183395.9

(51) Int. Cl. C12N 15/113; A61K 48/00; A61P 25/28

(52) U.S. Cl. CPC C12N 15/1138; A61K 48/005; A61P 25/28; C12N 2310/11; C12N 2310/322; C12N 2310/351

(57) ABSTRACT

The present disclosure concerns antisense oligonucleotide (ASO) capable of binding to a target site on the pre-mRNA of SORL1. The present disclosure further concerns a composition comprising said ASO. The present disclosure further concerns an ASO for use in medicine. The present disclosure further concerns an ASO for use in the prevention, treatment and/or alleviation of Alzheimer's Disease (AD), or a disease or disorder associated with Alzheimer's Disease. The present disclosure further concerns a method for mediating exon skipping in SORL1 transcripts, a method of determining the efficiency of ASO mediated SORL1 exon skipping, a method for testing if a patient identified with a SORL1 mutation will benefit from treatment with an ASO mediating exon skipping, and a method of producing an ASO.

### TECHNICAL FIELD

Disclosed herein are compounds, compositions and methods for modulating splicing of SORL1 mRNA in a cell, tissue or animal. Also provided are uses of disclosed compounds and compositions in the manufacture of a medicament for treatment of diseases and disorders, including Alzheimer's disease (AD). Specifically, the present disclosure relates to antisense oligonucleotides (ASOs) causing exon skipping in a SORL1 transcript.

### BACKGROUND

SORLA is important for Amyloid Precursor Protein (APP) transport out of the endosomes where, if not counteracted by SORLA, amyloidogenic processing of APP into pathogenic fragments (i.e. the Amyloid β-peptide (Aβ)) occurs. This SORLA-assisted transport of APP ensures a decreased cleavage of APP by the β-secretase, thereby reducing the production of the β-C-terminal fragment (CTF) that can subsequently be further processed to generate amyloid beta (Aβ) peptides.

In AD, Aβ accumulates in amyloid plaques within the brain, and is the most important pathological hallmark of the disease. However, the etiology of the disease is rather linked to the level of β-CTF, and other cargo proteins, that in AD cannot be recycled out of the endosome, leading to endosomal swelling and dysfunctional endosomal activity (i.e. the Endosomal Traffic Jam hypothesis for Alzheimer's disease).

The ability of SORLA to engage in endosomal recycling is linked to a motif in its cytoplasmic tail (i.e. the FANSHY motif) that is important for interaction with the retromer complex and which assists to traffic cargo out of endosomes.

The SORL1 gene—encoding the endosomal sorting receptor SORLA—has been associated with the development of Alzheimer's disease during the last 15 years. More recently, large whole-exome sequencing studies have identified how SORL1 is the gene harbouring the most genetic variation across the human genome in groups of AD patients.

The combined group of “loss-of-function” (LOF) variants located in SORL1 is linked with a 36-fold increased Odds Ratio (OR=36) risk of early-onset AD and 7-fold increased risk of late-onset AD. The overall group of missense variants has been found to be associated with a 2.7-fold and 1.9-fold increased risk of early-onset and late-onset AD, respectively.

Interestingly, SORL1 variants from patients with Alzheimer's disease spread across the entire SORL1 gene, and thus >25% of all variants locate to the genomic region encoding the eleven complement-type repeat (CR) domains. CR-domains represent the main ligand-binding site in all known receptors that contain clusters of CR-domains. Also the cluster of CR-domains of SORLA is involved in binding to ligands, incl. APP. Consequently, mutations in CR domains can have grave consequences on the functionality of SORLA, both with regard to ligand binding but also with regard to misfolding and ER retention of the protein.

While knowledge has been gained with regard genetic markers predicting a risk or causal connection for developing AD, no feasible treatment for AD is available to date and AD remains to be an immense burden to patients and the health care system.

### SUMMARY

Recently, the inventors of the present disclosure have gathered evidence that additional SORLA mutations, namely mutations in the CR domains of SORLA are associated with AD. Two subgroups of missense variants that locate in the region of SORL1 encoding the cluster of eleven CR-domains have been identified, which are associated with an increased risk of AD (data not published).

CR-domains represent the main ligand-binding site in all known receptors that contain clusters of CR-domains. Also the cluster of CR-domains of SORLA is involved in binding to ligands, incl. APP. However, the typical binding of any ligand does not depend on any isolated CR-domain, but many studies have rather shown how binding is achieved by combined interaction of a number of CR-domains with several epitopes on their ligand.

The inventors of the present disclosure have, by carefully studying SORLA domains and their functionality in physiology and pathology, realized that it is a feasible approach to target single or more SORLA CR domains with the aim to remove CR domains that are non-functional due to mutations, since the presence or absence of individual CR-domains has only subtle effects on the affinity for SORLA ligands. As disclosed herein, mutated CR domains are removed from SORLA by employing an exon-skipping approach, where specifically designed antisense oligonucleotides (ASOs) are used to remove exons of CR domains carrying a mutation. As a consequence, a functional SORLA protein, lacking for example one CR domain, or in the case of mutations in several CR domains, lacking more than one CR domain, can be produced. This approach has several advantages.

Firstly, ASO's delivered to the brain of AD patients can restore functional SORLA protein in situ. AD patients carrying CR-mutations may be producing SORLA protein, however, due to the mutation this protein is not functional. For example, this mutated SORLA protein can miss-fold and can get pathologically retained in the endoplasmatic reticulum (ER). By removing the mutated CR exon by exon skipping, functional SORLA protein can be produced that retains its functionality with regard to ligand binding, as well as ensuring that SORLA, comprising of many important domains, can proceed through the endosomal pathway in a physiological manner. In summary, instead of a mutated protein that completely abolishes the functionality of the whole protein, a variant lacking one or more CR domains can be produced, this variant being able to function physiologically or near-physiologically.

Secondly, mutated SORLA protein can have a dominant negative effect on non-mutated SORLA (produced from a non-affected allele) due to SORLA dimer formation. The mutated SORLA may lead to misfolding and retention of the non-mutated SORLA in the dimer. As such, the level of functional SORLA would be even more reduced, and as a consequence amyloidogenic processing of APP into pathogenic fragments cannot be counteracted any longer. However, by using the teaching of the present disclosure, i.e. ASO mediated exon-skipping of mutated SORLA CR domains, SORLA protein lacking one or more CR domains will be produced in the cell, and this variant will not induce misfolding neither of itself or the other unaffected SORLA variant in the dimer.

In a main aspect, the present disclosure concerns an antisense oligonucleotide (ASO) that binds to and/or is complementary to a target site on the pre-mRNA of SORL1, wherein the nucleotide sequence of the target site is comprised in a nucleotide sequence selected from the group consisting of
SEQ ID NO: 36, SEQ ID NO: 37, SEQ ID NO: 38, SEQ ID NO: 39,
SEQ ID NO: 40, SEQ ID NO: 41, SEQ ID NO: 42, SEQ ID NO: 43,
SEQ ID NO: 44, SEQ ID NO: 45 and SEQ ID NO: 46,
or a nucleotide sequence having at least 80% sequence identity or homology to a nucleotide sequence selected from the group consisting of
SEQ ID NO: 36, SEQ ID NO: 37, SEQ ID NO: 38, SEQ ID NO: 39,
SEQ ID NO: 40, SEQ ID NO: 41, SEQ ID NO: 42, SEQ ID NO: 43,
SEQ ID NO: 44, SEQ ID NO: 45 and SEQ ID NO: 46,
for example at least 80%, 85%, 90%, 95%, 98% or 99% sequence identity or homology thereto.

In a further aspect, the present disclosure concerns a composition comprising said oligonucleotide.

In a further aspect, the present disclosure is directed to said antisense oligonucleotide (ASO) and/or said composition, for use in medicine.

In a further aspect, the present disclosure is directed to said antisense oligonucleotide (ASO) and/or said composition, for use in the prevention, treatment and/or alleviation of Alzheimer's Disease (AD), or a disease or disorder associated with Alzheimer's Disease.

In a further aspect, the present disclosure is directed the use of said antisense oligonucleotide or said composition in the manufacture of a medicament for the treatment Alzheimer's disease (AD), or a disease or disorder associated with Alzheimer's Disease.

In a further aspect, the present disclosure is directed to a method for mediating exon skipping in SORL1 transcripts in a cell, tissue or organ using said antisense oligonucleotide or/or said composition,
where the exon is selected from the group consisting of exon 23, exon 24, exon 25, exon 26, exon 27, exon 28, exon 29, exon 30, exon 31, exon 32 and exon 33 of SORL1.

In a further aspect, the present disclosure is directed to a method of determining the efficiency of ASO mediated SORL1 exon skipping in a subject, the method comprising the following steps:
a) analyzing the levels of shed SORLA in a first sample comprising cerebrospinal fluid derived from a patient, obtained before treatment with an ASO,
b) analyzing the levels of shed SORLA in a second sample comprising cerebrospinal fluid derived from the same patient as in a), obtained after treatment with an ASO,
c) comparing the level of shed SORLA in the samples of a) and b),
   thereby determining exon skipping if the level of shed SORLA in b) is higher than in a).

In a further aspect, the present disclosure is directed to a method for testing if a patient identified with a SORL1 mutation will benefit from treatment with an ASO mediating exon skipping, the method comprising the steps of:
a) identifying a mutation in any one of exon 23 to 33 of SORL1,
b) introducing the SORL1 mutation identified in the patient into a cell line,
c) selecting one or more ASO that targets the exon carrying the identified mutation,
d) contacting a first aliquot of cells of with medium comprising the selected one or more ASO, and contacting a second aliquot of cells of with medium not comprising any ASO,
e) analyzing the levels of shed SORLA in the first and the second aliquot,
f) comparing the level of shed SORLA in the first and the second aliquot,
thereby determining that the patient will benefit from treatment with the one or more ASO if the level of shed SORLA in the first aliquot is higher than in the second aliquot.

In a further aspect, the present disclosure is directed to a method of producing an ASO suitable for treatment of a patient with AD, wherein the patient carries a mutation in an exon encoding a complement-type repeat (CR) domain of SORLA, the method comprising the following steps:
a) identifying, for example in silico identifying, an ASO according to any one of claims 1 to 21,
b) determining if the target site of the ASO comprises the mutation, or if the target size of the ASO does not comprise the mutation, and
   thereby determining that the ASO that binds to a target site not comprising the mutation is suitable for treatment of a patient with AD.

### DESCRIPTION OF DRAWINGS

FIG. 1: Schematic representation of SORLA domain assembly

The human SORLA polypeptide contains 2214 amino acids that folds into a number of protein domains, including a VPS10p-domain, a YWTD-b-propeller-domain connected with an EGF-domain, eleven CR-domains, six 3Fn-domains, a transmembrane domain, and a cytoplasmic tail domain. The CR-domains are encoded by Exons 23-33). (Ex=Exon)

FIG. 2: SORLA CR-domain structure and sequence

A and B: CR-domain sequences contain approximately 40 amino acids including six strictly conserved cysteines (cysteine=“C”) that form three intradomain disulfides (black bars connecting cysteines). Four residues with acidic side chains are also conserved and they function in octahedral coordination of a calcium ion. CR1 here depicts an exemplary CR-domain.

C: Alignment of the eleven CR-domain sequences of SORLA, with domain boundaries following their individual exon structures (i.e. exons 23-33). The numbers on top of the alignment indicate the relation between the amino acids in B and C.

FIG. 3: Schematics how antisense oligonucleotides (ASO) can be used for exon skipping

Each of the eleven CR-domains is coded by its own exon (exons 23-33). Each of these exons contains multiples of three nucleotides, and skipping of an exon does therefore not affect the reading frame of downstream exons. ASO (depicted as wavy lines) for individual exons, targeting the 3′ splice site, the 5′ splice site and/or one or more exonic splice enhancer sites (ESE), can accordingly be used to cure Alzheimer's disease by removing the mutated exon from SORL1 transcripts, as here exemplified by exon 23.

FIG. 4: Schematics for ASO treatment and cellular SORLA activity

A) SORLA expressed from wildtype alleles has eleven CR-domains (11×) and functions in endosomal cargo recycling.

B) SORLA expressed from an allele with a pathogenic SORL1 variant (indicated as black CR domain), e.g. a variant characteristic for an AD patient, e.g. a variant of the ONC or CC type, the variant causing receptor misfolding and ER-retention, which potentially also affects the translation product from the wildtype allele.

C) SORLA from disease-alleles treated with exon-skipping ASO will contain ten functional CR-domains (10×) and functions indistinguishable from the full-length SORLA protein.

FIG. 5: ASOs targeting exon 23, exon 27, exon 33

A) Targeting strategy for inducing exon skipping of exon 23. The table shows four ASOs (ASO23.1-ASO23.4), the respective ASO sequence and the respective RNA target sequence.

B) Targeting strategy for inducing exon skipping of exon 27. The table shows four ASOs (ASO27.1-ASO27.4), the respective ASO sequence and the respective RNA target sequence.

C) Targeting strategy for inducing exon skipping of exon 33. The table shows four ASOs (ASO33.1-ASO33.4), the respective ASO sequence and the respective RNA target sequence.

D) Four identified ASOs for targeting exon 23, in relation to their respective binding sites on the precursor mRNA. Bars in different shades of grey indicate binding sites for splice factors.

E) Four identified ASOs for targeting exon 27, in relation to their respective binding sites on the precursor mRNA. Bars in different shades of grey indicate binding sites for splice factors.

F) Four identified ASOs for targeting exon 33, in relation to their respective binding sites on the precursor mRNA. Bars in different shades of grey indicate binding sites for splice factors.

In D)-F), the width of each bar represents the sequence putatively bound by a splicing factor. The predicted strength to promote or repress splicing of an exon is expressed as a score for each splicing factor. A positive score represents an exon splice enhancers (ESE), while a negative score represents an exon splice repressors ESR). The numbers below the sequence indicate the nucleotide position within the sequence comprising the exon plus 25 bp flanking intron sequence (lowercase) on each side.

FIG. 6: Exon 23 skipping efficiency measured by RT-PCR on human cell lines transfected with exon 23 targeting ASOs

We tested 4 ASOs (ASO23.1, ASO23.2, ASO23.3, and ASO23.4) directed against exon splice enhancer (ESE) elements of SORL1 exon 23, by transfection of HEK293 cells, harvesting endogenous SORL1 mRNA and did RT-PCR using a primer pair spanning the region around exon 23. PCR products were separated by agarose gel electrophoresis, and data from two independent experiments are shown (upper and lower figure). In both experiments we observed clear evidence that ASO23.2 and ASO23.3 induced skipping of exon 23, as demonstrated by the presence of a shorter PCR-product (see arrow) that migrates identical to products generated using a plasmid encoding the exon 23-deleted fragment (pΔEx23, a recombinantly produced variant lacking exon 23 used as control) as template. pFL (plasmid encoding full-length SORLA) served as control template to identify PCR-products corresponding to fragments that have exon 23 included.

FIG. 7: SORL1 delta-Exon23 expression

A) Western blot analysis of lysates and medium from N2a cells transfected with construct encoding SORLA full-length or SORLA-delta-Exon23. The levels of full length SORLA in lysate (left) is indistinguishable from the levels of SORLA without Exon 23 (note: the band in the second replicate of delta Ex23 is partly covered by an air bubble), but nevertheless as intense as the bands in the neighbouring lanes. The levels of full length SORLA in medium (right) is indistinguishable from the levels of SORLA without Exon 23. Thus, SOLRLA without exon 23 is shed similar to full length SORLA, and as such retains its functionality.

B) Western blot analysis of lysates and medium from N2a cells transfected with SORLA full-length, SORLA-D1105H (pathogenic mutation), SORLA without exon 23, or SORLA-R1080C (pathogenic mutation). The level of sSORLA in medium of deleted CR1 (i.e. Delta exon 23) is similar as for full-length SORLA, whereas both pathogenic mutations strongly reduce the amount of sSORLA production (sSORLA=shed SORLA).

FIG. 8: Function expression of SORLA delta-Exon23

A) Western blot analysis of medium from N2a cells transfected with APP alone (“control”, lanes 1-2), double-transfected with APP/full-length SORLA (“full-length”, lanes 3-4), or double-transfected with APP/SORLA deltaEx23 (“delta Exon23”, lanes 5-6). Amount of shed APP (i.e. sAPPa) is detected using antibody WO2 and amount of shed SORLA (i.e. sSORLA) is detected using a polyclonal serum for the SORLA luminal fragment.

Cells with no exogenous SORLA overexpression (control) show a strong signal for sAPP in the medium. In contrast, medium from cells transfected with either full-length

SORLA or the SORLA construct with deletion of exon 23 show strongly reduced levels of sAPP. Conclusively, SORLA deleted of CR1 (encoded by exon 23) is as efficient in reducing APP processing as is the full-length SORLA receptor.

B-C) Independent experiments again showed that the level of shed SORLA (sSORLA) is similar between SORLA-WT and SORLA-ΔEx23, showing that deletion of CR1 (encoded by Exon 23) has no observable impact on receptor biology.

We also again observed that the two SORLA variants (full-length and SORLA-delta-Exon23) protein have indistinguishable effect on lowering sAPPα production by decreasing APP proteolysis.

D) Blots from three independent replicates were quantified, and data presented as means of duplicate samples with levels relative to cells with no exogenous SORLA.

The quantification further shows that SORLA ΔEx23 is as effective as WT to decrease shed APPa (sAPPa) (ns=non-significant).

FIG. 9: SORL1 delta-Exon33 expression

N2a cells were transfected with constructs for either SORL1-WT or SORL1-ΔEx33 and lysates and conditioned medium from cells were analysed by Western blotting using antibodies for SORLA or Actin (in lysate samples) or shed SORLA (sSORLA) (in medium samples).

Expression of endogenous SORLA is low/absent in N2a cells and as such not detectable in non-transfected cells (blank). SORLA lacking CR domain 11 (encoded by Exon 33 which is omitted from construct SORL1-ΔEx33) was detected at similar levels in lysates and in medium compared to wildtype SORLA. Similar detection in medium indicates that deletion of CR11 has no observable impact on SORLA receptor biology, i.e. even SORLA lacking CR11 is processed and shed similarly to SORLA-WT.

FIG. 10: Exon-skipping technology expanded to additional SORL1 CR-domain exons

HEK293 cells were transfected with constructs engineered to generate SORLA proteins deleted for individual CR-domains, i.e. CR1 (ΔEx23), CR2 (ΔEx24), CR3 (ΔEx25), CR4 (ΔEx26), CR5 (ΔEx27), CR6 (ΔEx28), CR7+8 (ΔEx29+30), CR8 (ΔEx30), CR9 (ΔE31), CR10 (ΔE32) or CR11 (ΔE33). Lysates were prepared from cells harvested 72 hours post-transfection, proteins separated by 26-lane SDS-PAGE NuPAGE system, and analysed by Western blotting analysis with a polyclonal SORLA serum from rabbit (sol-SORLA).

SORLA blots are known to show double bands when expressing SORLA in HEK cells, with an upper band (running slower in the gel due to a larger molecular size) representing mature SORLA, and a lower band (running faster in the gel due to a smaller molecular size; see arrows).

Each of the construct led to expression of a specific, CR-domain-deleted SORLA receptor. Interestingly, some deletions showed a surprising result suggesting that potentially not all CR-domains can be deleted without disturbance of receptor function, i.e. CR4 (ΔE26) and CR9 (ΔEx31) where the SORLA double band pattern is disturbed (*).

FL=full-length SORLA, Δ=delta indicating omitted exon/domain, Ex=exon, CR.

### DETAILED DESCRIPTION

### Definitions

As used herein, the singular forms “a”, “an” and “the” include plural referents unless the context clearly states otherwise.

The term “some embodiments” can include one, or more than one embodiment.

The terms “Alzheimer's disease” and “AD” are used interchangeably throughout the description.

The use of the word “a” or “an” when used throughout the text or in conjunction with the term “comprising” in the claims and/or the specification may mean “one,” but it is also consistent with the meaning of “one or more,” “at least one,” and “one or more than one.” Thus, for example, reference to “ASO” includes a plurality of such ASOs, such as one or more ASOs, at least one ASOs, or two or more ASOs.

The term “SORLA” as used herein is synonymous to the terms SORLA, Sortilin-related receptor, sortilin related receptor 1, SORL1, Low-density lipoprotein receptor relative with 11 ligand-binding repeats, LDLR relative with 11 ligand-binding repeats, LR11, SorLA-1, Sorting protein-related receptor containing LDLR class A repeats and gp250. Human sorLA is annotated in UniProt under the accession number Q92673.

The terms homology, identity and similarity, with respect to a polynucleotide (or polypeptide), as defined herein are used interchangeably and refer to the percentage of nucleic acids (or amino acids) in the candidate sequence that are, homolog, identical or similar, respectively, to the residues of a corresponding native nucleic acids (or amino acids), after aligning the sequences and introducing gaps, if necessary, to achieve the maximum percent identity/similarity, and considering any conservative substitutions according to the NCIUB rules (hftp://www.chem.qmul.ac.uk/iubmb/misc/naseq.html; NC-IUB, Eur J Biochem (1985)) as part of the sequence identity. In particular, the percentage of similarity refers to the percentage of residues conserved with similar physiochemical properties. Neither 5′ or 3′ extensions nor insertions (for nucleic acids) or N′ or C′ extensions nor insertions (for polypeptides) result in a reduction of identity or similarity. Methods and computer programs for the alignments are well known in the art. Generally, a given similarity between two sequences implies that the identity between these sequences is at least equal to the similarity; for example, if two sequences are 80% similar to one another, they cannot be less than 80% identical to one another—but could be sharing 90% identity.

As defined herein the term “at least 80% homology, similarity or identity” means at least 85%, at least 90%, at least 95%, at least 98% or at least 99% homology, similarity or identity throughout the present disclosure.

### ASO Mediated Exon-Skipping

Antisense oligonucleotides can be used to induce exon-skipping in the pre-mRNA transcripts (also referred to as precursor mRNA). This type of antisense-mediated splicing modulation uses antisense oligonucleotides (ASOs) to manipulate the splicing, for example by sterically blocking the binding of splicing factors to pre-mRNA transcripts (also referred to as precursor mRNA).

In a main aspect, the present disclosure concerns an antisense oligonucleotide (ASO) that binds to and/or is complementary to a target site on the pre-mRNA of SORL1, wherein the nucleotide sequence of the target site is comprised in a nucleotide sequence selected from the group consisting of
SEQ ID NO: 36, SEQ ID NO: 37, SEQ ID NO: 38, SEQ ID NO: 39,
SEQ ID NO: 40, SEQ ID NO: 41, SEQ ID NO: 42, SEQ ID NO: 43,
SEQ ID NO: 44, SEQ ID NO: 45 and SEQ ID NO: 46,
or a nucleotide sequence having at least 80% sequence identity or homology to a nucleotide sequence selected from the group consisting of
SEQ ID NO: 36, SEQ ID NO: 37, SEQ ID NO: 38, SEQ ID NO: 39,
SEQ ID NO: 40, SEQ ID NO: 41, SEQ ID NO: 42, SEQ ID NO: 43,
SEQ ID NO: 44, SEQ ID NO: 45 and SEQ ID NO: 46,
for example at least 80%, 85%, 90%, 95%, 98% or 99% sequence identity or homology thereto.

In an alternative aspect, the present disclosure concerns an antisense oligonucleotide (ASO) capable of binding to a target site on the pre-mRNA of SORL1, wherein the nucleotide sequence of the target site is comprised in a nucleotide sequence selected from the group consisting of
SEQ ID NO: 36, SEQ ID NO: 37, SEQ ID NO: 38, SEQ ID NO: 39,
SEQ ID NO: 40, SEQ ID NO: 41, SEQ ID NO: 42, SEQ ID NO: 43,
SEQ ID NO: 44, SEQ ID NO: 45 and SEQ ID NO: 46,
or a nucleotide sequence having at least 80% sequence identity or homology to a nucleotide sequence selected from the group consisting of
SEQ ID NO: 36, SEQ ID NO: 37, SEQ ID NO: 38, SEQ ID NO: 39,
SEQ ID NO: 40, SEQ ID NO: 41, SEQ ID NO: 42, SEQ ID NO: 43,
SEQ ID NO: 44, SEQ ID NO: 45 and SEQ ID NO: 46,
for example at least 80%, 85%, 90%, 95%, 98% or 99% sequence identity or homology thereto.

The person skilled in the art will appreciate that sufficient binding of an ASO to a target site requires binding via complementary bases.

The person skilled in the art will appreciate that a pre-mRNA of SORL1 is understood as a transcript from one or more SORL1 exons with or without additional nucleotides as transcribed from upstream and/or downstream sequences flanking the respective exon(s). For example, the pre-mRNA of SORL1 may comprise an
Exon 23 transcript comprising 100 nucleotides transcribed from the upstream/downstream flanking intron (SEQ ID NO: 36),
Exon 24 transcript comprising 100 nucleotides transcribed from the upstream/downstream flanking intron (SEQ ID NO: 37),
Exon 25 transcript comprising 100 nucleotides transcribed from the upstream/downstream flanking intron (SEQ ID NO: 38),
Exon 26 transcript comprising 100 nucleotides transcribed from the upstream/downstream flanking intron (SEQ ID NO: 39),
Exon 27 transcript comprising 100 nucleotides transcribed from the upstream/downstream flanking intron (SEQ ID NO: 40),
Exon 28 transcript comprising 100 nucleotides transcribed from the upstream/downstream flanking intron (SEQ ID NO: 41),
Exon 29 transcript comprising 100 nucleotides transcribed from the upstream/downstream flanking intron (SEQ ID NO: 42),
Exon 30 transcript comprising 100 nucleotides transcribed from the upstream/downstream flanking intron (SEQ ID NO: 43),
Exon 31 transcript comprising 100 nucleotides transcribed from the upstream/downstream flanking intron (SEQ ID NO: 44),
Exon 32 transcript comprising 100 nucleotides transcribed from the upstream/downstream flanking intron (SEQ ID NO: 45), or an
Exon 33 transcript comprising 100 nucleotides transcribed from the upstream/downstream flanking intron (SEQ ID NO: 46),
   wherein exon 23 encodes SORLA CR-domain 1, exon 24 encodes SORLA CR-domain 2, exon 25 encodes SORLA CR-domain 3, exon 26 encodes SORLA CR-domain 4, exon 27 encodes SORLA CR-domain 5, exon 28 encodes SORLA CR-domain 6, exon 29 encodes SORLA CR-domain 7, exon 30 encodes SORLA CR-domain 8, exon 31 encodes SORLA CR-domain 9, exon 32 encodes SORLA CR-domain 10 and exon 33 encodes SORLA CR-domain 11.

In some embodiments of the present disclosure, the nucleotide sequence of the target site comprises a nucleotide sequence of SEQ ID NO: 36, or a nucleotide sequence having at least 80% sequence identity or homology to SEQ ID NO: 36 for example at least 80%, 85%, 90%, 95%, 98% or 99% sequence identity or homology thereto.

In some embodiments of the present disclosure, the nucleotide sequence of the target site comprises a nucleotide sequence of SEQ ID NO: 37, or a nucleotide sequence having at least 80% sequence identity or homology to SEQ ID NO: 37 for example at least 80%, 85%, 90%, 95%, 98% or 99% sequence identity or homology thereto.

In some embodiments of the present disclosure, the nucleotide sequence of the target site comprises a nucleotide sequence of SEQ ID NO: 38, or a nucleotide sequence having at least 80% sequence identity or homology to SEQ ID NO: 38 for example at least 80%, 85%, 90%, 95%, 98% or 99% sequence identity or homology thereto.

In some embodiments of the present disclosure, the nucleotide sequence of the target site comprises a nucleotide sequence of SEQ ID NO: 39, or a nucleotide sequence having at least 80% sequence identity or homology to SEQ ID NO: 39 for example at least 80%, 85%, 90%, 95%, 98% or 99% sequence identity or homology thereto.

In some embodiments of the present disclosure, the nucleotide sequence of the target site comprises a nucleotide sequence of SEQ ID NO: 40, or a nucleotide sequence having at least 80% sequence identity or homology to SEQ ID NO: 40 for example at least 80%, 85%, 90%, 95%, 98% or 99% sequence identity or homology thereto.

In some embodiments of the present disclosure, the nucleotide sequence of the target site comprises a nucleotide sequence of SEQ ID NO: 41, or a nucleotide sequence having at least 80% sequence identity or homology to SEQ ID NO: 41 for example at least 80%, 85%, 90%, 95%, 98% or 99% sequence identity or homology thereto.

In some embodiments of the present disclosure, the nucleotide sequence of the target site comprises a nucleotide sequence of SEQ ID NO: 42, or a nucleotide sequence having at least 80% sequence identity or homology to SEQ ID NO: 42 for example at least 80%, 85%, 90%, 95%, 98% or 99% sequence identity or homology thereto.

In some embodiments of the present disclosure, the nucleotide sequence of the target site comprises a nucleotide sequence of SEQ ID NO: 43, or a nucleotide sequence having at least 80% sequence identity or homology to SEQ ID NO: 43 for example at least 80%, 85%, 90%, 95%, 98% or 99% sequence identity or homology thereto.

In some embodiments of the present disclosure, the nucleotide sequence of the target site comprises a nucleotide sequence of SEQ ID NO: 44, or a nucleotide sequence having at least 80% sequence identity or homology to SEQ ID NO: 44 for example at least 80%, 85%, 90%, 95%, 98% or 99% sequence identity or homology thereto.

In some embodiments of the present disclosure, the nucleotide sequence of the target site comprises a nucleotide sequence of SEQ ID NO: 45, or a nucleotide sequence having at least 80% sequence identity or homology to SEQ ID NO: 45 for example at least 80%, 85%, 90%, 95%, 98% or 99% sequence identity or homology thereto.

In some embodiments of the present disclosure, the nucleotide sequence of the target site comprises a nucleotide sequence of SEQ ID NO: 46, or a nucleotide sequence having at least 80% sequence identity or homology to SEQ ID NO: 46 for example at least 80%, 85%, 90%, 95%, 98% or 99% sequence identity or homology thereto.

In some embodiments of the present disclosure, the oligonucleotide binding to and/or complementary to the target site causes exon skipping of an exon encoding a complement-type repeat (CR) domains of SORLA, wherein the exon comprises or consists of a nucleotide sequence selected from the group consisting of
exon 23 as set forth in SEQ ID NO: 1,
exon 24 as set forth in SEQ ID NO: 2,
exon 25 as set forth in SEQ ID NO: 3,
exon 26 as set forth in SEQ ID NO: 4,
exon 27 as set forth in SEQ ID NO: 5,
exon 28 as set forth in SEQ ID NO: 6,
exon 29 as set forth in SEQ ID NO: 7,
exon 30 as set forth in SEQ ID NO: 8,
exon 31 as set forth in SEQ ID NO: 9,
exon 32 as set forth in SEQ ID NO: 10, and
exon 33 as set forth in SEQ ID NO: 11,
   or a nucleotide sequence having at least 80% sequence identity or homology to any one of SEQ ID NO: 1 to SEQ ID NO: 11, for example at least 80%, 85%, 90%, 95%, 98% or 99% sequence identity or homology thereto.

In some embodiments of the present disclosure, the exon comprises or consists of a nucleotide sequence selected from the group consisting of
exon 23 as set forth in SEQ ID NO: 1,
exon 24 as set forth in SEQ ID NO: 2,
exon 25 as set forth in SEQ ID NO: 3,
exon 26 as set forth in SEQ ID NO: 4,
exon 27 as set forth in SEQ ID NO: 5,
exon 28 as set forth in SEQ ID NO: 6,
exon 29 as set forth in SEQ ID NO: 7,
exon 30 as set forth in SEQ ID NO: 8,
exon 31 as set forth in SEQ ID NO: 9,
exon 32 as set forth in SEQ ID NO: 10, and
exon 33 as set forth in SEQ ID NO: 11,
   or a nucleotide sequence having at least 80% sequence identity or homology to any one of SEQ ID NO: 1 to SEQ ID NO: 11, for example at least 80%, 85%, 90%, 95%, 98% or 99% sequence identity or homology thereto.

In some embodiments of the present disclosure, the target site causes exon skipping of an exon encoding a complement-type repeat (CR) domains of SORLA, wherein the exon comprises or consists of a nucleotide sequence consisting of exon 23 as set forth in SEQ ID NO: 1.

In some embodiments of the present disclosure, the target site causes exon skipping of an exon encoding a complement-type repeat (CR) domains of SORLA, wherein the exon comprises or consists of a nucleotide sequence consisting of exon 24 as set forth in SEQ ID NO: 2.

In some embodiments of the present disclosure, the target site causes exon skipping of an exon encoding a complement-type repeat (CR) domains of SORLA, wherein the exon comprises or consists of a nucleotide sequence consisting of exon 25 as set forth in SEQ ID NO: 3.

In some embodiments of the present disclosure, the target site causes exon skipping of an exon encoding a complement-type repeat (CR) domains of SORLA, wherein the exon comprises or consists of a nucleotide sequence consisting of exon 26 as set forth in SEQ ID NO: 4.

In some embodiments of the present disclosure, the target site causes exon skipping of an exon encoding a complement-type repeat (CR) domains of SORLA, wherein the exon comprises or consists of a nucleotide sequence consisting of exon 27 as set forth in SEQ ID NO: 5.

In some embodiments of the present disclosure, the target site causes exon skipping of an exon encoding a complement-type repeat (CR) domains of SORLA, wherein the exon comprises or consists of a nucleotide sequence consisting of exon 28 as set forth in SEQ ID NO: 6.

In some embodiments of the present disclosure, the target site causes exon skipping of an exon encoding a complement-type repeat (CR) domains of SORLA, wherein the exon comprises or consists of a nucleotide sequence consisting of exon 29 as set forth in SEQ ID NO: 7.

In some embodiments of the present disclosure, the target site causes exon skipping of an exon encoding a complement-type repeat (CR) domains of SORLA, wherein the exon comprises or consists of a nucleotide sequence consisting of exon 30 as set forth in SEQ ID NO: 8.

In some embodiments of the present disclosure, the target site causes exon skipping of an exon encoding a complement-type repeat (CR) domains of SORLA, wherein the exon comprises or consists of a nucleotide sequence consisting of exon 31 as set forth in SEQ ID NO: 9.

In some embodiments of the present disclosure, the target site causes exon skipping of an exon encoding a complement-type repeat (CR) domains of SORLA, wherein the exon comprises or consists of a nucleotide sequence consisting of exon 32 as set forth in SEQ ID NO: 10.

In some embodiments of the present disclosure, the target site causes exon skipping of an exon encoding a complement-type repeat (CR) domains of SORLA, wherein the exon comprises or consists of a nucleotide sequence consisting of exon 33 as set forth in SEQ ID NO: 11.

In some embodiments of the present disclosure, the oligonucleotide is between 10 to 30 nucleotides in length, such as 10 to 13 nucleotides, such as 10 to 16 nucleotides, such as 10 to 19 nucleotides, such as 10 to 22 nucleotides, such as 10 to 23 nucleotides, such as 10 to 26 nucleotides, such as 10 to 29 nucleotides, such as 10 to 30 nucleotides.

In some embodiments of the present disclosure, the oligonucleotide is at least 10 nucleotides long, such as at least 12 nucleotides, and/or at least 14 nucleotides, and/or at least 16 nucleotides and/or at least 18 nucleotides, and/or at least 20, and/or at least 22 nucleotides, and/or at least 24 nucleotides, and/or at least 26 nucleotides, and/or at least 28 nucleotides, and/or at least 30 nucleotides long.

In some embodiments of the present disclosure, the oligonucleotide is 21 nucleotides long.

In some embodiments of the present disclosure, the oligonucleotide has a GC-content of 40 to 60%, such as 45 to 55%.

In some embodiments of the present disclosure, the oligonucleotide comprises a backbone comprising of phosphorothioate (PS).

In some embodiments of the present disclosure, the oligonucleotide further comprises modifications at at least one nucleotide position, or at each nucleotide position.

In some embodiments of the present disclosure, the modification is a modification of the nucleic acid backbone, the nucleobase, the ribose sugar and/or 2′-ribose substitutions.

In some embodiments of the present disclosure, the oligonucleotide comprises a 2′-O-methoxyethyl sugar modification.

In some embodiments of the present disclosure, the oligonucleotide comprises a 2′-O-methy ribose modification.

In some embodiments of the present disclosure, the target site is at the 3′ splice site boundary, at the 5′ splice site boundary and/or at an exonic splice enhancer (ESE) site.

In some embodiments of the present disclosure, the target site consists or comprises of a nucleotide sequence selected from the group consisting of
SEQ ID NO: 12, SEQ ID NO: 13, SEQ ID NO: 14, SEQ ID NO: 15,
SEQ ID NO: 20, SEQ ID NO: 21, SEQ ID NO: 22, SEQ ID NO: 23,
SEQ ID NO: 28, SEQ ID NO: 29, SEQ ID NO: 30, SEQ ID NO: 31,
SEQ ID NO: 36, SEQ ID NO: 37, SEQ ID NO: 38, SEQ ID NO: 39,
SEQ ID NO: 40, SEQ ID NO: 41, SEQ ID NO: 42, SEQ ID NO: 43,
SEQ ID NO: 44, SEQ ID NO: 45 and SEQ ID NO: 46,
or a nucleotide sequence having at least 80% sequence identity or homology to a nucleotide sequence selected from the group consisting of
SEQ ID NO: 12, SEQ ID NO: 13, SEQ ID NO: 14, SEQ ID NO: 15,
SEQ ID NO: 20, SEQ ID NO: 21, SEQ ID NO: 22, SEQ ID NO: 23,
SEQ ID NO: 28, SEQ ID NO: 29, SEQ ID NO: 30, SEQ ID NO: 31,
SEQ ID NO: 36, SEQ ID NO: 37, SEQ ID NO: 38, SEQ ID NO: 39,
SEQ ID NO: 40, SEQ ID NO: 41, SEQ ID NO: 42, SEQ ID NO: 43,
SEQ ID NO: 44, SEQ ID NO: 45 and SEQ ID NO: 46,
for example at least 80%, 85%, 90%, 95%, 98% or 99% sequence identity or homology thereto.

In some embodiments of the present disclosure, the target site consists or comprises of a nucleotide sequence as set forth in SEQ ID NO: 12, or a nucleotide sequence having at least 80% sequence identity or homology to SEQ ID NO: 12 for example at least 80%, 85%, 90%, 95%, 98% or 99% sequence identity or homology thereto.

In some embodiments of the present disclosure, the target site consists or comprises of a nucleotide sequence as set forth in SEQ ID NO: 13, or a nucleotide sequence having at least 80% sequence identity or homology to SEQ ID NO: 13 for example at least 80%, 85%, 90%, 95%, 98% or 99% sequence identity or homology thereto.

In some embodiments of the present disclosure, the target site consists or comprises of a nucleotide sequence as set forth in SEQ ID NO: 14, or a nucleotide sequence having at least 80% sequence identity or homology to SEQ ID NO: 14 for example at least 80%, 85%, 90%, 95%, 98% or 99% sequence identity or homology thereto.

In some embodiments of the present disclosure, the target site consists or comprises of a nucleotide sequence as set forth in SEQ ID NO: 15, or a nucleotide sequence having at least 80% sequence identity or homology to SEQ ID NO: 15 for example at least 80%, 85%, 90%, 95%, 98% or 99% sequence identity or homology thereto.

In some embodiments of the present disclosure, the target site consists or comprises of a nucleotide sequence as set forth in SEQ ID NO: 20, or a nucleotide sequence having at least 80% sequence identity or homology to SEQ ID NO: 20 for example at least 80%, 85%, 90%, 95%, 98% or 99% sequence identity or homology thereto.

In some embodiments of the present disclosure, the target site consists or comprises of a nucleotide sequence as set forth in SEQ ID NO: 21, or a nucleotide sequence having at least 80% sequence identity or homology to SEQ ID NO: 21 for example at least 80%, 85%, 90%, 95%, 98% or 99% sequence identity or homology thereto.

In some embodiments of the present disclosure, the target site consists or comprises of a nucleotide sequence as set forth in SEQ ID NO: 22, or a nucleotide sequence having at least 80% sequence identity or homology to SEQ ID NO: 22 for example at least 80%, 85%, 90%, 95%, 98% or 99% sequence identity or homology thereto.

In some embodiments of the present disclosure, the target site consists or comprises of a nucleotide sequence as set forth in SEQ ID NO: 23, or a nucleotide sequence having at least 80% sequence identity or homology to SEQ ID NO: 23 for example at least 80%, 85%, 90%, 95%, 98% or 99% sequence identity or homology thereto.

In some embodiments of the present disclosure, the target site consists or comprises of a nucleotide sequence as set forth in SEQ ID NO: 28, or a nucleotide sequence having at least 80% sequence identity or homology to SEQ ID NO: 28 for example at least 80%, 85%, 90%, 95%, 98% or 99% sequence identity or homology thereto.

In some embodiments of the present disclosure, the target site consists or comprises of a nucleotide sequence as set forth in SEQ ID NO: 29, or a nucleotide sequence having at least 80% sequence identity or homology to SEQ ID NO: 29 for example at least 80%, 85%, 90%, 95%, 98% or 99% sequence identity or homology thereto.

In some embodiments of the present disclosure, the target site consists or comprises of a nucleotide sequence as set forth in SEQ ID NO: 30, or a nucleotide sequence having at least 80% sequence identity or homology to SEQ ID NO: 30 for example at least 80%, 85%, 90%, 95%, 98% or 99% sequence identity or homology thereto.

In some embodiments of the present disclosure, the target site consists or comprises of a nucleotide sequence as set forth in SEQ ID NO: 31, or a nucleotide sequence having at least 80% sequence identity or homology to SEQ ID NO: 31 for example at least 80%, 85%, 90%, 95%, 98% or 99% sequence identity or homology thereto.

In some embodiments of the present disclosure, the target site consists or comprises of a nucleotide sequence as set forth in SEQ ID NO: 36, or a nucleotide sequence having at least 80% sequence identity or homology to SEQ ID NO: 36 for example at least 80%, 85%, 90%, 95%, 98% or 99% sequence identity or homology thereto.

In some embodiments of the present disclosure, the target site consists or comprises of a nucleotide sequence as set forth in SEQ ID NO 37, or a nucleotide sequence having at least 80% sequence identity or homology to SEQ ID NO: 37 for example at least 80%, 85%, 90%, 95%, 98% or 99% sequence identity or homology thereto.

In some embodiments of the present disclosure, the target site consists or comprises of a nucleotide sequence as set forth in SEQ ID NO: 38, or a nucleotide sequence having at least 80% sequence identity or homology to SEQ ID NO: 38 for example at least 80%, 85%, 90%, 95%, 98% or 99% sequence identity or homology thereto.

In some embodiments of the present disclosure, the target site consists or comprises of a nucleotide sequence as set forth in SEQ ID NO: 39, or a nucleotide sequence having at least 80% sequence identity or homology to SEQ ID NO: 39 for example at least 80%, 85%, 90%, 95%, 98% or 99% sequence identity or homology thereto.

In some embodiments of the present disclosure, the target site consists or comprises of a nucleotide sequence as set forth in SEQ ID NO: 40, or a nucleotide sequence having at least 80% sequence identity or homology to SEQ ID NO: 40 for example at least 80%, 85%, 90%, 95%, 98% or 99% sequence identity or homology thereto.

In some embodiments of the present disclosure, the target site consists or comprises of a nucleotide sequence as set forth in SEQ ID NO: 41, or a nucleotide sequence having at least 80% sequence identity or homology to SEQ ID NO: 41 for example at least 80%, 85%, 90%, 95%, 98% or 99% sequence identity or homology thereto.

In some embodiments of the present disclosure, the target site consists or comprises of a nucleotide sequence as set forth in SEQ ID NO: 42, or a nucleotide sequence having at least 80% sequence identity or homology to SEQ ID NO: 42 for example at least 80%, 85%, 90%, 95%, 98% or 99% sequence identity or homology thereto.

In some embodiments of the present disclosure, the target site consists or comprises of a nucleotide sequence as set forth in SEQ ID NO: 43, or a nucleotide sequence having at least 80% sequence identity or homology to SEQ ID NO: 43 for example at least 80%, 85%, 90%, 95%, 98% or 99% sequence identity or homology thereto.

In some embodiments of the present disclosure, the target site consists or comprises of a nucleotide sequence as set forth in SEQ ID NO: 44, or a nucleotide sequence having at least 80% sequence identity or homology to SEQ ID NO: 44 for example at least 80%, 85%, 90%, 95%, 98% or 99% sequence identity or homology thereto.

In some embodiments of the present disclosure, the target site consists or comprises of a nucleotide sequence as set forth in SEQ ID NO: 45, or a nucleotide sequence having at least 80% sequence identity or homology to SEQ ID NO: 45 for example at least 80%, 85%, 90%, 95%, 98% or 99% sequence identity or homology thereto.

In some embodiments of the present disclosure, the target site consists or comprises of a nucleotide sequence as set forth in SEQ ID NO: 46, or a nucleotide sequence having at least 80% sequence identity or homology to SEQ ID NO: 46 for example at least 80%, 85%, 90%, 95%, 98% or 99% sequence identity or homology thereto.

In some embodiments of the present disclosure, the oligonucleotide consists or comprises of a nucleotide sequence selected from the group consisting of
SEQ ID NO: 16, SEQ ID NO: 17, SEQ ID NO: 18, SEQ ID NO: 19,
SEQ ID NO: 24, SEQ ID NO: 25, SEQ ID NO: 26, SEQ ID NO: 27,
SEQ ID NO: 32, SEQ ID NO: 33, SEQ ID NO: 34 and SEQ ID NO: 35
or a nucleotide sequence having at least 80% sequence identity or homology to a nucleotide sequence selected from the group consisting of
SEQ ID NO: 16, SEQ ID NO: 17, SEQ ID NO: 18, SEQ ID NO: 19,
SEQ ID NO: 24, SEQ ID NO: 25, SEQ ID NO: 26, SEQ ID NO: 27,
SEQ ID NO: 32, SEQ ID NO: 33, SEQ ID NO: 34 and SEQ ID NO: 35,
for example at least 80%, 85%, 90%, 95%, 98% or 99% sequence identity or homology thereto.

In some embodiments of the present disclosure, the oligonucleotide consists or comprises of a nucleotide sequence as set forth in SEQ ID NO: 16, or a nucleotide sequence having at least 80% sequence identity or homology to SEQ ID NO: 16, for example at least 80%, 85%, 90%, 95%, 98% or 99% sequence identity or homology thereto.

In some embodiments of the present disclosure, the oligonucleotide consists or comprises of a nucleotide sequence as set forth in SEQ ID NO: 17, or a nucleotide sequence having at least 80% sequence identity or homology to SEQ ID NO: 17, for example at least 80%, 85%, 90%, 95%, 98% or 99% sequence identity or homology thereto.

In some embodiments of the present disclosure, the oligonucleotide consists or comprises of a nucleotide sequence as set forth in SEQ ID NO: 18, or a nucleotide sequence having at least 80% sequence identity or homology to SEQ ID NO: 18, for example at least 80%, 85%, 90%, 95%, 98% or 99% sequence identity or homology thereto.

In some embodiments of the present disclosure, the oligonucleotide consists or comprises of a nucleotide sequence as set forth in SEQ ID NO: 19, or a nucleotide sequence having at least 80% sequence identity or homology to SEQ ID NO: 19, for example at least 80%, 85%, 90%, 95%, 98% or 99% sequence identity or homology thereto.

In some embodiments of the present disclosure, the oligonucleotide consists or comprises of a nucleotide sequence as set forth in SEQ ID NO: 24, or a nucleotide sequence having at least 80% sequence identity or homology to SEQ ID NO: 24, for example at least 80%, 85%, 90%, 95%, 98% or 99% sequence identity or homology thereto.

In some embodiments of the present disclosure, the oligonucleotide consists or comprises of a nucleotide sequence as set forth in SEQ ID NO: 25, or a nucleotide sequence having at least 80% sequence identity or homology to SEQ ID NO: 25, for example at least 80%, 85%, 90%, 95%, 98% or 99% sequence identity or homology thereto.

In some embodiments of the present disclosure, the oligonucleotide consists or comprises of a nucleotide sequence as set forth in SEQ ID NO: 26, or a nucleotide sequence having at least 80% sequence identity or homology to SEQ ID NO: 26, for example at least 80%, 85%, 90%, 95%, 98% or 99% sequence identity or homology thereto.

In some embodiments of the present disclosure, the oligonucleotide consists or comprises of a nucleotide sequence as set forth in SEQ ID NO: 27, or a nucleotide sequence having at least 80% sequence identity or homology to SEQ ID NO: 27, for example at least 80%, 85%, 90%, 95%, 98% or 99% sequence identity or homology thereto.

In some embodiments of the present disclosure, the oligonucleotide consists or comprises of a nucleotide sequence as set forth in SEQ ID NO: 32, or a nucleotide sequence having at least 80% sequence identity or homology to SEQ ID NO: 32, for example at least 80%, 85%, 90%, 95%, 98% or 99% sequence identity or homology thereto.

In some embodiments of the present disclosure, the oligonucleotide consists or comprises of a nucleotide sequence as set forth in SEQ ID NO: 33, or a nucleotide sequence having at least 80% sequence identity or homology to SEQ ID NO: 33, for example at least 80%, 85%, 90%, 95%, 98% or 99% sequence identity or homology thereto.

In some embodiments of the present disclosure, the oligonucleotide consists or comprises of a nucleotide sequence as set forth in SEQ ID NO: 34, or a nucleotide sequence having at least 80% sequence identity or homology to SEQ ID NO: 34, for example at least 80%, 85%, 90%, 95%, 98% or 99% sequence identity or homology thereto.

In some embodiments of the present disclosure, the oligonucleotide consists or comprises of a nucleotide sequence as set forth in SEQ ID NO: 35, or a nucleotide sequence having at least 80% sequence identity or homology to SEQ ID NO: 35, for example at least 80%, 85%, 90%, 95%, 98% or 99% sequence identity or homology thereto.

In some embodiments of the present disclosure, the oligonucleotide hybridizes specifically under high stringency solution hybridization conditions to target site.

In some embodiments of the present disclosure, upon binding to the target site, the oligonucleotide prevents splicing factors from binding.

In some embodiments of the present disclosure, the oligonucleotide is conjugated to a moiety or to a nanoparticle formulation.

In some embodiments of the present disclosure, the moiety is a cell-targeting moiety and/or a cell-penetrating moiety.

In some embodiments of the present disclosure, the oligonucleotide is conjugated to Triantennary N-acetylgalactosamine (GalNAc) moiety and/or a peptide.

In some embodiments of the present disclosure, the oligonucleotide is targeted to a 5′ splice site, a 3′ splice site and/or an exonic splice enhancer site (ESE).

The oligonucleotide according to any one of the preceding claims, wherein the oligonucleotide further comprises at least one additional nucleotide, at least two additional nucleotides, at least three additional nucleotides, at one or both ends of the oligonucleotide.

In a further aspect, the present disclosure is directed to a composition comprising said oligonucleotide.

In some embodiments of the present disclosure, the composition is a pharmaceutical composition.

In some embodiments of the present disclosure, the composition further comprises a pharmaceutically acceptable carrier.

In some embodiments of the present disclosure, the composition comprises one or more of said oligonucleotides.

In some embodiments of the present disclosure, the composition comprises the ASO as set forth in SEQ ID NO: 16 and SEQ ID NO: 17.

In some embodiments of the present disclosure, the composition comprises the ASO as set forth in SEQ ID NO: 16 and SEQ ID NO: 18.

In some embodiments of the present disclosure, the composition comprises the ASO as set forth in SEQ ID NO: 16 and SEQ ID NO: 19.

In some embodiments of the present disclosure, the composition comprises the ASO as set forth in SEQ ID NO: 17 and SEQ ID NO: 18.

In some embodiments of the present disclosure, the composition comprises the ASO as set forth in SEQ ID NO: 17 and SEQ ID NO: 19.

In some embodiments of the present disclosure, the composition comprises the ASO as set forth in SEQ ID NO: 18 and SEQ ID NO: 19.

In some embodiments of the present disclosure, the composition comprises the ASO as set forth in SEQ ID NO: 16 and SEQ ID NO: 17 and SEQ ID NO: 18.

In some embodiments of the present disclosure, the composition comprises the ASO as set forth in SEQ ID NO: 16 and SEQ ID NO: 18 and SEQ ID NO: 19.

In some embodiments of the present disclosure, the composition comprises the ASO as set forth in SEQ ID NO: 17 and SEQ ID NO: 18 and SEQ ID NO: 19.

In some embodiments of the present disclosure, the composition comprises the ASO as set forth in SEQ ID NO: 16 and SEQ ID NO: 17 and SEQ ID NO: 18 and SEQ ID NO: 19.

In some embodiments of the present disclosure, the composition comprises the ASO as set forth in SEQ ID NO: 24 and SEQ ID NO: 25.

In some embodiments of the present disclosure, the composition comprises the ASO as set forth in SEQ ID NO: 24 and SEQ ID NO: 26.

In some embodiments of the present disclosure, the composition comprises the ASO as set forth in SEQ ID NO: 24 and SEQ ID NO: 27.

In some embodiments of the present disclosure, the composition comprises the ASO as set forth in SEQ ID NO: 25 and SEQ ID NO: 26.

In some embodiments of the present disclosure, the composition comprises the ASO as set forth in SEQ ID NO: 25 and SEQ ID NO: 27.

In some embodiments of the present disclosure, the composition comprises the ASO as set forth in SEQ ID NO: 26 and SEQ ID NO: 27.

In some embodiments of the present disclosure, the composition comprises the ASO as set forth in SEQ ID NO: 24 and SEQ ID NO: 25 and SEQ ID NO: 26.

In some embodiments of the present disclosure, the composition comprises the ASO as set forth in SEQ ID NO: 24 and SEQ ID NO: 26 and SEQ ID NO: 27.

In some embodiments of the present disclosure, the composition comprises the ASO as set forth in SEQ ID NO: 25 and SEQ ID NO: 26 and SEQ ID NO: 27.

In some embodiments of the present disclosure, the composition comprises the ASO as set forth in SEQ ID NO: 24 and SEQ ID NO: 25 and SEQ ID NO: 26 and SEQ ID NO: 27.

In some embodiments of the present disclosure, the composition comprises the ASO as set forth in SEQ ID NO: 32 and SEQ ID NO: 33.

In some embodiments of the present disclosure, the composition comprises the ASO as set forth in SEQ ID NO: 32 and SEQ ID NO: 34.

In some embodiments of the present disclosure, the composition comprises the ASO as set forth in SEQ ID NO: 32 and SEQ ID NO: 35.

In some embodiments of the present disclosure, the composition comprises the ASO as set forth in SEQ ID NO: 33 and SEQ ID NO: 34.

In some embodiments of the present disclosure, the composition comprises the ASO as set forth in SEQ ID NO: 33 and SEQ ID NO: 35.

In some embodiments of the present disclosure, the composition comprises the ASO as set forth in SEQ ID NO: 34 and SEQ ID NO: 35.

In some embodiments of the present disclosure, the composition comprises the ASO as set forth in SEQ ID NO: 32 and SEQ ID NO: 33 and SEQ ID NO: 34.

In some embodiments of the present disclosure, the composition comprises the ASO as set forth in SEQ ID NO: 32 and SEQ ID NO: 34 and SEQ ID NO: 35.

In some embodiments of the present disclosure, the composition comprises the ASO as set forth in SEQ ID NO: 33 and SEQ ID NO: 34 and SEQ ID NO: 35.

In some embodiments of the present disclosure, the composition comprises the ASO as set forth in SEQ ID NO: 32 and SEQ ID NO: 33 and SEQ ID NO: 34 and SEQ ID NO: 35.

In a further aspect, the present disclosure is directed to said antisense oligonucleotide (ASO) and/or said composition, for use in medicine.

In a further aspect, the present disclosure is directed to said antisense oligonucleotide (ASO) and/or said composition, for use in the prevention, treatment and/or alleviation of Alzheimer's Disease (AD), or a disease or disorder associated with Alzheimer's Disease.

The person skilled in the art will appreciate that the herein disclosed invention can be used to prevent AD and/or treat AD and/or alleviate symptoms of AD in AD patients carrying pathologic mutations in SORLA CR-domains. AD is a devastative disease affecting the brain on a structural level. It may therefore be beneficial to provide ASO mediating exon-skipping of pathologically mutated CR domains at an early stage, e.g. prior to the onset of symptoms, or at an early AD stage, or prior to substantive structural brain remodeling. For example, the herein described approach would be applicable to treat family members of AD patients prior to disease development.

In some embodiments of the present disclosure, the one or more ASOs of the present invention, or a composition comprising the one or more ASOs of the present invention are administered to an individual prior to the onset of AD symptoms.

In some embodiments of the present disclosure, the one or more ASOs of the present invention, or a composition comprising the one or more ASOs of the present invention are administered to an individual prior to the onset of AD symptoms.

In some embodiments of the present disclosure, the one or more ASOs of the present invention, or a composition comprising the one or more ASOs of the present invention are administered to an individual when a family member is diagnosed with AD.

Alternatively, the herein disclosed invention would be applicable to treat patients with early symptoms.

In some embodiments of the present disclosure, the one or more ASOs of the present invention, or a composition comprising the one or more ASOs of the present invention are administered to an individual with early AD symptoms.

Alternatively, the herein disclosed invention would be applicable to treat patients at various disease stages of AD.

In some embodiments of the present disclosure, the one or more ASOs of the present invention, or a composition comprising the one or more ASOs of the present invention are administered to an individual with a varying degree of AD symptoms.

Further, the herein disclosed invention would, due to its nature of restoring physiologic SORLA function, by applicable to treat patients at late stages of AD.

In some embodiments of the present disclosure, the one or more ASOs of the present invention, or a composition comprising the one or more ASOs of the present invention are administered to a patient at late stage of AD.

In some embodiments of the present disclosure, an effective amount of the antisense oligonucleotide is administered to the eye, to the spinal cord, to cerebrospinal fluid, to the brain and/or to the liver.

In some embodiments of the present disclosure, an effective amount of the antisense oligonucleotide is administered to an individual when a relative of said individual is diagnosed with Alzheimer's disease.

In some embodiments of the present disclosure, an effective amount of the antisense oligonucleotide is administered to an individual when it becomes known that a relative of said individual is suffering or has suffered from Alzheimer's disease.

It may, for example, become known that a relative of an individual is suffering from AD when said relative is diagnosed by a clinician. Alternatively, it may, for example become known that a relative of said individual is suffering or has suffered from Alzheimer's disease by obtaining information from other sources, such as family records or memories.

A relative of an individual may be understood as a family member of an individual.

A relative of an individual may be understood as a person sharing genetic material with said individual.

For example, a relative of an individual is a great-grandmother.

For example, a relative of an individual is a great-grandfather.

For example, a relative of an individual is a grandmother.

For example, a relative of an individual is a grandfather.

For example, a relative of an individual is a mother.

For example, a relative of an individual is a father.

For example, a relative of an individual is a sibling.

For example, a relative of an individual is a brother.

For example, a relative of an individual is a daughter.

For example, a relative of an individual is a son.

In some embodiments of the present disclosure, an effective amount of the antisense oligonucleotide is administered to an individual once it is established that one or more family members of said individual suffer from Alzheimer's disease.

In some embodiments of the present disclosure, an effective amount of the antisense oligonucleotide is administered to an individual once it is established that one or more family members of said individual suffer from Alzheimer's disease.

The person skilled in the art will appreciate a genetic inherence of a risk to develop AD.

The person skilled in the art will appreciate that administration of an antisense oligonucleotide of the invention to an individual at risk for developing AD, e.g. having a risk when being a relative of a person diagnosed with AD, will offer, for example, the possibility of preventing AD prior to disease onset, or of reducing symptoms of AD.

In a further aspect, the present disclosure is directed the use of said antisense oligonucleotide or said composition in the manufacture of a medicament for the treatment Alzheimer's disease (AD), or a disease or disorder associated with Alzheimer's Disease.

In another aspect, the present disclosure is directed the use of said antisense oligonucleotide for the preparation of a medicament for treating Alzheimer's Disease (AD), or a disease or disorder associated with Alzheimer's Disease.

In a further aspect, the present disclosure is directed to a method for mediating exon skipping in SORL1 transcripts in a cell, tissue or organ using said antisense oligonucleotide (ASO) and/or said composition,
where the exon is selected from the group consisting of exon 23, exon 24, exon 25, exon 26, exon 27, exon 28, exon 29, exon 30, exon 31, exon 32 and exon 33 of SORL1.

In some embodiments of the present disclosure, one ASO is used in said method.

In some embodiments of the present disclosure, more than one ASO is used in said method, for example two ASOs, for example three ASOs, for example four ASOs, for example five ASOs, for example six ASOs.

In some embodiments of the present disclosure, the ASOs used in said method are the ASOs as set forth in SEQ ID NO: 16 and SEQ ID NO: 17.

In some embodiments of the present disclosure, the ASOs used in said method are the ASOs as set forth in SEQ ID NO: 16 and SEQ ID NO: 18.

In some embodiments of the present disclosure, the ASOs used in said method are the ASOs as set forth in SEQ ID NO: 16 and SEQ ID NO: 19.

In some embodiments of the present disclosure, the ASOs used in said method are the ASOs as set forth in SEQ ID NO: 17 and SEQ ID NO: 18.

In some embodiments of the present disclosure, the ASOs used in said method are the ASOs as set forth in SEQ ID NO: 17 and SEQ ID NO: 19.

In some embodiments of the present disclosure, the ASOs used in said method are the ASOs as set forth in SEQ ID NO: 18 and SEQ ID NO: 19.

In some embodiments of the present disclosure, the composition comprises the ASO as set forth in SEQ ID NO: 16 and SEQ ID NO: 17 and SEQ ID NO: 18.

In some embodiments of the present disclosure, the ASOs used in said method are the ASOs as set forth in SEQ ID NO: 16 and SEQ ID NO: 18 and SEQ ID NO: 19.

In some embodiments of the present disclosure, the ASOs used in said method are the ASOs as set forth in SEQ ID NO: 17 and SEQ ID NO: 18 and SEQ ID NO: 19.

In some embodiments of the present disclosure, the ASOs used in said method are the ASOs as set forth in SEQ ID NO: 16 and SEQ ID NO: 17 and SEQ ID NO: 18 and SEQ ID NO: 19.

In some embodiments of the present disclosure, the ASOs used in said method are the ASOs as set forth in SEQ ID NO: 24 and SEQ ID NO: 25.

In some embodiments of the present disclosure, the ASOs used in said method are the ASOs as set forth in SEQ ID NO: 24 and SEQ ID NO: 26.

In some embodiments of the present disclosure, the ASOs used in said method are the ASOs as set forth in SEQ ID NO: 24 and SEQ ID NO: 27.

In some embodiments of the present disclosure, the ASOs used in said method are the ASOs as set forth in SEQ ID NO: 25 and SEQ ID NO: 26.

In some embodiments of the present disclosure, the ASOs used in said method are the ASOs as set forth in SEQ ID NO: 25 and SEQ ID NO: 27.

In some embodiments of the present disclosure, the ASOs used in said method are the ASOs as set forth in SEQ ID NO: 26 and SEQ ID NO: 27.

In some embodiments of the present disclosure, the ASOs used in said method are the ASOs as set forth in SEQ ID NO: 24 and SEQ ID NO: 25 and SEQ ID NO: 26.

In some embodiments of the present disclosure, the ASOs used in said method are the ASOs as set forth in SEQ ID NO: 24 and SEQ ID NO: 26 and SEQ ID NO: 27.

In some embodiments of the present disclosure, the ASOs used in said method are the ASOs as set forth in SEQ ID NO: 25 and SEQ ID NO: 26 and SEQ ID NO: 27.

In some embodiments of the present disclosure, the ASOs used in said method are the ASOs as set forth in SEQ ID NO: 24 and SEQ ID NO: 25 and SEQ ID NO: 26 and SEQ ID NO: 27.

In some embodiments of the present disclosure, the ASOs used in said method are the ASOs as set forth in SEQ ID NO: 32 and SEQ ID NO: 33.

In some embodiments of the present disclosure, the ASOs used in said method are the ASOs as set forth in SEQ ID NO: 32 and SEQ ID NO: 34.

In some embodiments of the present disclosure, the ASOs used in said method are the ASOs as set forth in SEQ ID NO: 32 and SEQ ID NO: 35.

In some embodiments of the present disclosure, the ASOs used in said method are the ASOs as set forth in SEQ ID NO: 33 and SEQ ID NO: 34.

In some embodiments of the present disclosure, the ASOs used in said method are the ASOs as set forth in SEQ ID NO: 33 and SEQ ID NO: 35.

In some embodiments of the present disclosure, the ASOs used in said method are the ASOs as set forth in SEQ ID NO: 34 and SEQ ID NO: 35.

In some embodiments of the present disclosure, the ASOs used in said method are the ASOs as set forth in SEQ ID NO: 32 and SEQ ID NO: 33 and SEQ ID NO: 34.

In some embodiments of the present disclosure, the ASOs used in said method are the ASOs as set forth in SEQ ID NO: 32 and SEQ ID NO: 34 and SEQ ID NO: 35.

In some embodiments of the present disclosure, the ASOs used in said method are the ASOs as set forth in SEQ ID NO: 33 and SEQ ID NO: 34 and SEQ ID NO: 35.

In some embodiments of the present disclosure, the ASOs used in said method are the ASOs as set forth in SEQ ID NO: 32 and SEQ ID NO: 33 and SEQ ID NO: 34 and SEQ ID NO: 35.

In some embodiments of the present disclosure, exon skipping of one exon is mediated by said method.

In some embodiments of the present disclosure, exon skipping of more than one exon is mediated by said method, for example two exons, for example three exons, for example four exons.

In an alternative further aspect, the present disclosure is directed to a method of determining the efficiency of ASO mediated SORL1 exon skipping in a subject, the method comprising the following steps:
a) analyzing the levels of shed SORLA in a first sample comprising cerebrospinal fluid derived from a patient, obtained before treatment with an ASO,
b) analyzing the levels of shed SORLA in a second sample comprising cerebrospinal fluid derived from the same patient as in a), obtained after treatment with an ASO,
c) comparing the level of shed SORLA in the samples of a) and b), and
thereby determining exon skipping if the level of shed SORLA in b) is higher than in a).

In an alternative aspect, the present disclosure is directed to a method of determining the efficiency of ASO mediated SORL1 exon skipping in a subject, the method comprising the following steps:
a) analyzing the levels of shed SORLA in a first sample comprising cerebrospinal fluid derived from a patient, obtained before treatment with an ASO,
b) analyzing the levels of shed SORLA in a second sample comprising cerebrospinal fluid derived from the same patient as in a), obtained after treatment with an ASO,
c) comparing the level of shed SORLA in the samples of a) and b), and
d) determining exon skipping if the level of shed SORLA in b) is higher than in a).

The person skilled in the art will understand, as also described in the Examples disclosed herein, that the level of shed SORLA may be used as an indicator of functional SORLA that can be physiologically processed in the cell and thus shed according to physiological processes. Non-functional SORLA which is, for example, misfolded, for example due to mutations in one or more CR-domains, will not be physiologically processed and will not be shed or will be shed to a lower degree. When functionality is restored, e.g. by the herein disclosed exon skipping approach using ASOs, thus omitting the mutated exon, processing of SORLA and shedding will be restored.

In some embodiments of the present disclosure, said method optionally comprises the step of obtaining sample b) at several time points after treatment with an ASO, thus monitoring the efficiency of ASO mediated exon skipping over time.

In a further aspect, the present disclosure is directed to a method for testing if a patient identified with a SORL1 mutation will benefit from treatment with an ASO mediating exon skipping, the method comprising the steps of:
a) identifying a mutation in any one of exon 23 to 33 of SORL1,
b) introducing the SORL1 mutation identified in the patient into a cell line,
c) selecting one or more ASO that targets the exon carrying the identified mutation,
d) contacting a first aliquot of cells of with medium comprising the selected one or more ASO, and contacting a second aliquot of cells of with medium not comprising any ASO,
e) analyzing the levels of shed SORLA in the first and the second aliquot,
f) comparing the level of shed SORLA in the first and the second aliquot,
thereby determining that the patient will benefit from treatment with the one or more ASO if the level of shed SORLA in the first aliquot is higher than in the second aliquot.

In some embodiments of the present disclosure, said method is an in-vitro method.

In a further aspect, the present disclosure is directed to a method of producing an ASO suitable for treatment of a patient with Alzheimer's disease (AD), wherein the patient carries a mutation in an exon encoding a complement-type repeat (CR) domain of SORLA, the method comprising the following steps:
a) identifying, for example in silico identifying, an ASO according to any one of claims 1 to 21,
b) determining if the target site of the ASO comprises the mutation, or if the target size of the ASO does not comprise the mutation, and
   thereby determining that the ASO that binds to a target site not comprising the mutation is suitable for treatment of a patient with AD.

The person skilled in the art will appreciate that an a mutation in a ASO target site may compromise effective binding of the ASO.

In some embodiments of the present disclosure, said mutation is a calcium-cage-mutation or an odd-numbered cysteines-mutation.

In some embodiments of the present disclosure, said mutation is a substitution of arginine to cysteine at position 1080 (R1080C) of human SORL1, or wherein the mutation is a substitution of aspartic acid to histidine at position 1105 (C1105H) of human SORL1

### EXAMPLES

### Example 1: Developing SORL1 Specific Exon-Skipping ASOs

### Aim:

To provide proof-of-concept we identified individual ASOs that induce or are candidates for inducing exon skipping of SORL1 exon 23, of SORL1 exon 27, and of SORL1 exon 33.

### Background:

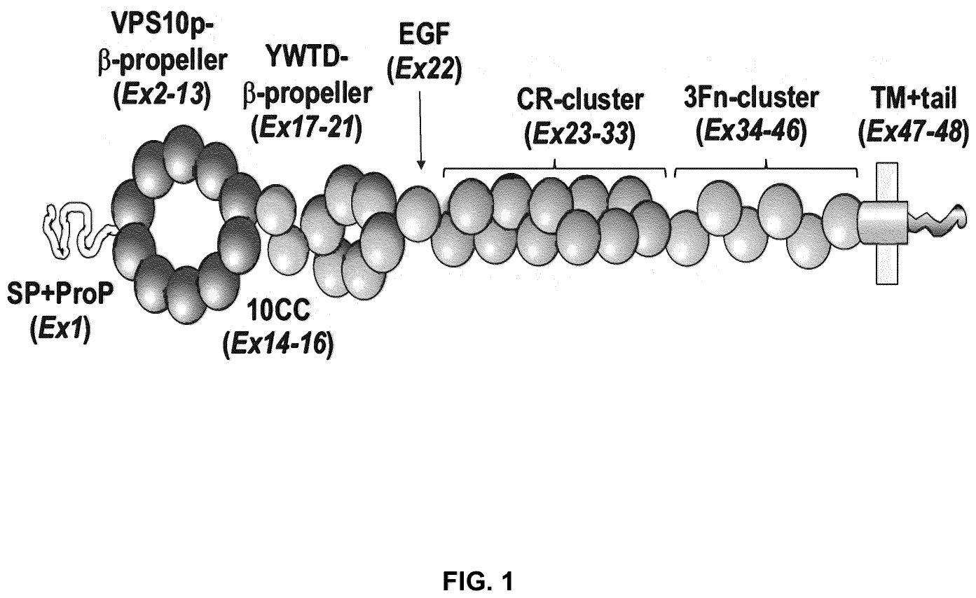

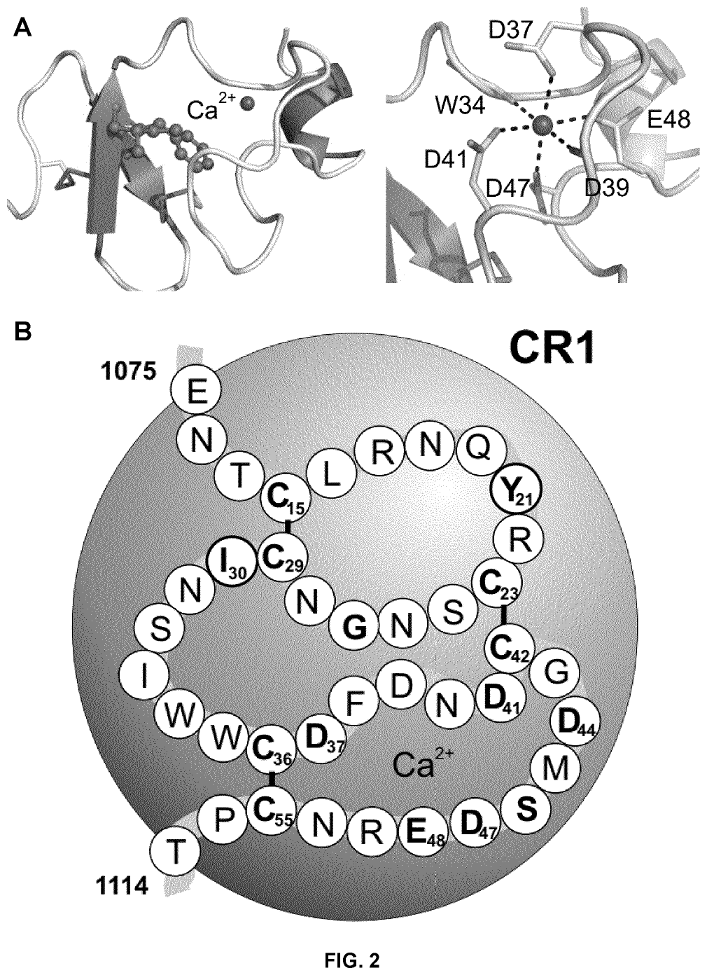

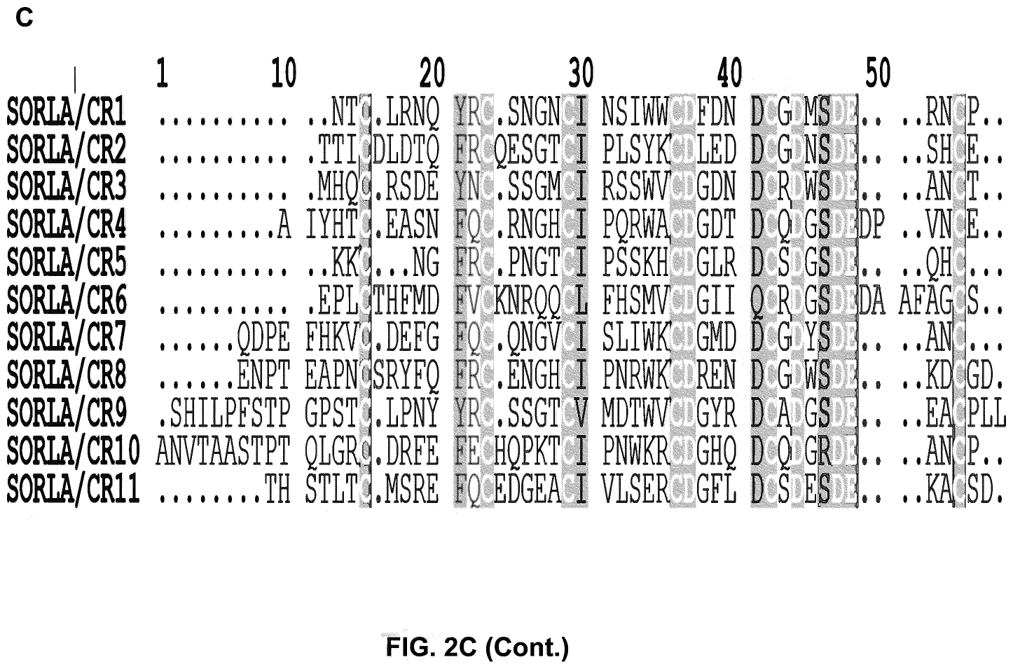

The human SORLA polypeptide contains 2214 amino acids that folds into a number of protein domains, including a VPS10p-domain, a YWTD-b-propeller-domain connected with an EGF-domain, eleven CR-domains, six 3Fn-domains, a transmembrane domain, and a cytoplasmic tail domain (FIG. 1). The CR-domains are encoded by Exons 23-33). CR-domain sequences contain approximately 40 amino acids including six strictly conserved cysteines that form three intradomain disulfides (FIGS. 2A and B). Four residues with acidic side chains are also conserved and they function in octahedral coordination of a calcium ion. FIG. 2C depicts an alignment of the eleven CR-domain sequences of SORLA, with domain boundaries following their individual exon structures (i.e. exons 23-33).

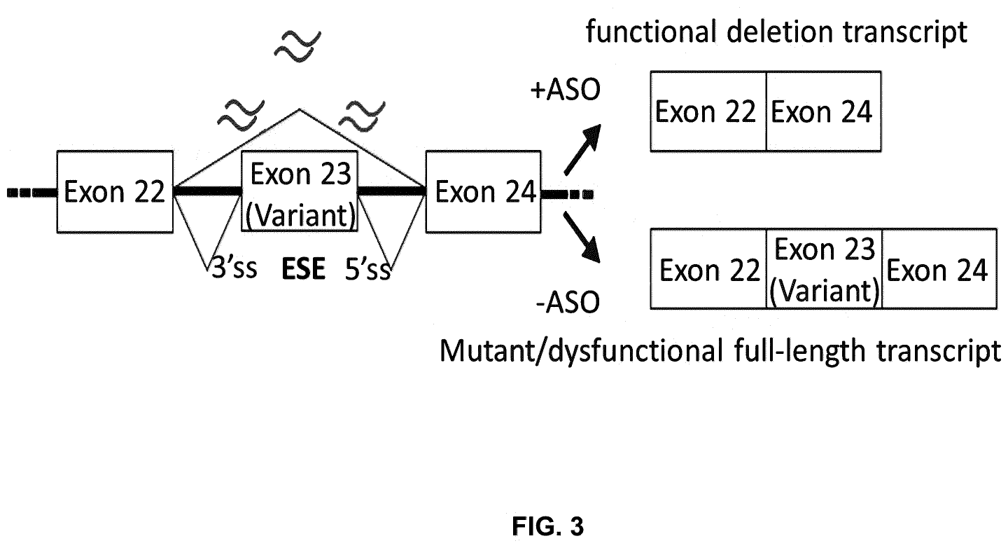

FIG. 3 is a schematics showing how antisense oligonucleotides (ASO) can be used for exon skipping. Each of the eleven CR-domains is coded by its own exon (exons 23-33). Each of these exons contains multiples of three nucleotides, and skipping of an exon does therefore not affect the reading frame of downstream exons. ASO for individual exons, targeting the 3′ splice site, the 5′ splice site and/or one or more exonic splice enhancer sites (ESE), can accordingly be used to cure Alzheimer's disease by removing the mutated exon from SORL1 transcripts, as here exemplified by exon 23.

Interestingly, SORL1 variants from patients with Alzheimer's disease spread across the entire SORL1 gene, and thus >25% of all variants locate to the genomic region encoding the eleven CR-domains (Holstege 2020).

We have recently analysed the distribution of ONC (odd-numbered cysteines) and CC (calcium cage) variants in CR-domain sequences (data not shown). We mapped SORL1 variants identified in AD patients of recently published exome sequencing data from AD patients and non-demented controls. Each of the eleven CR-domains harbour mutations from AD patients.

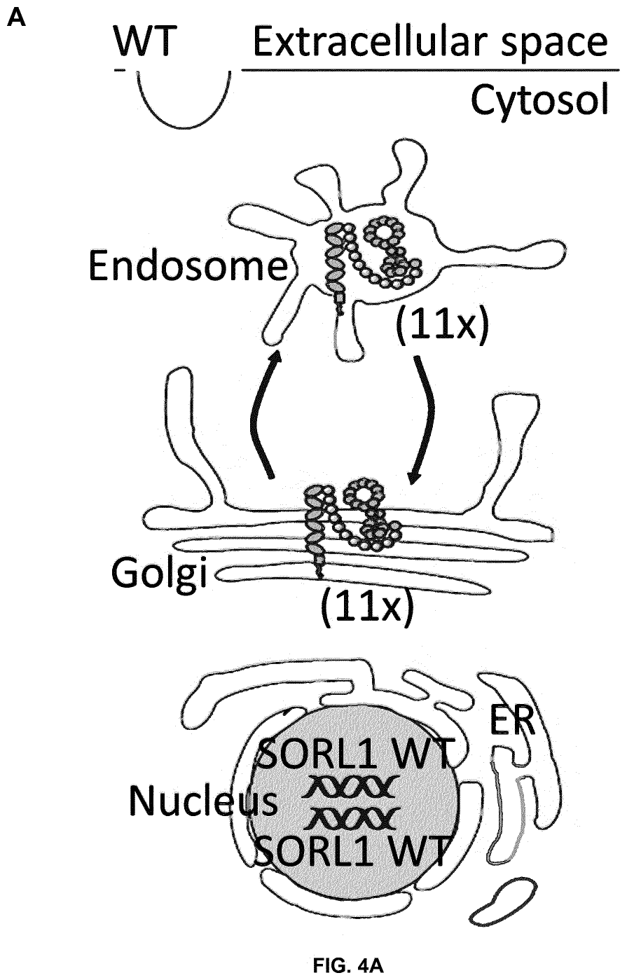

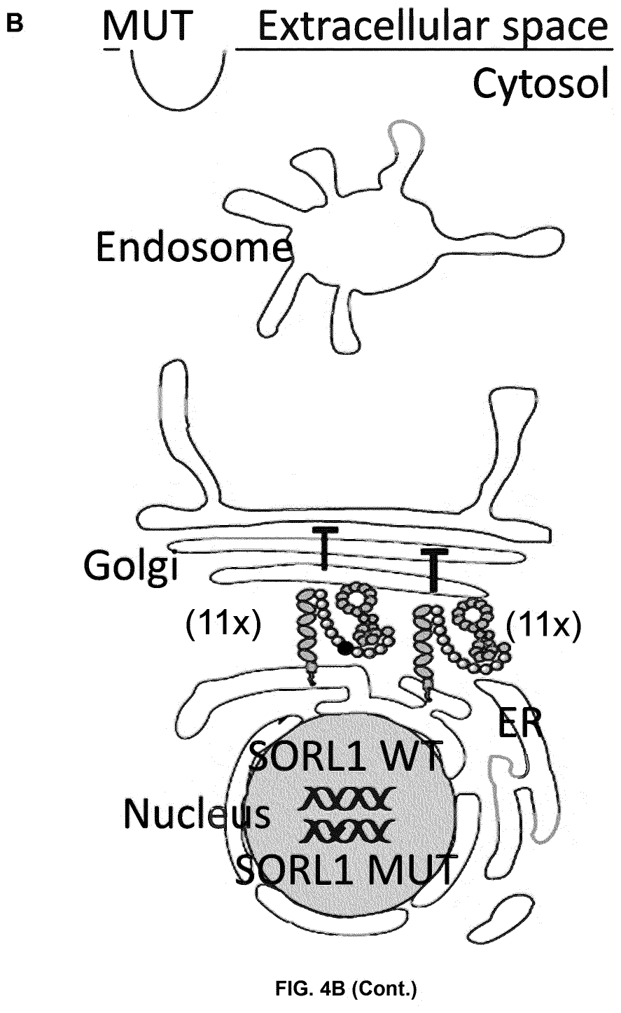

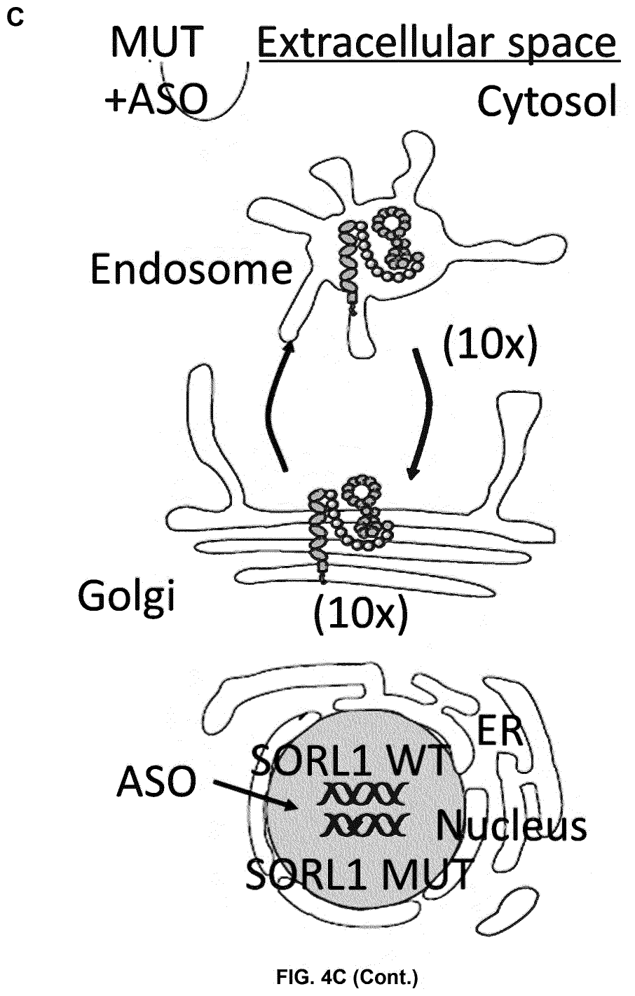

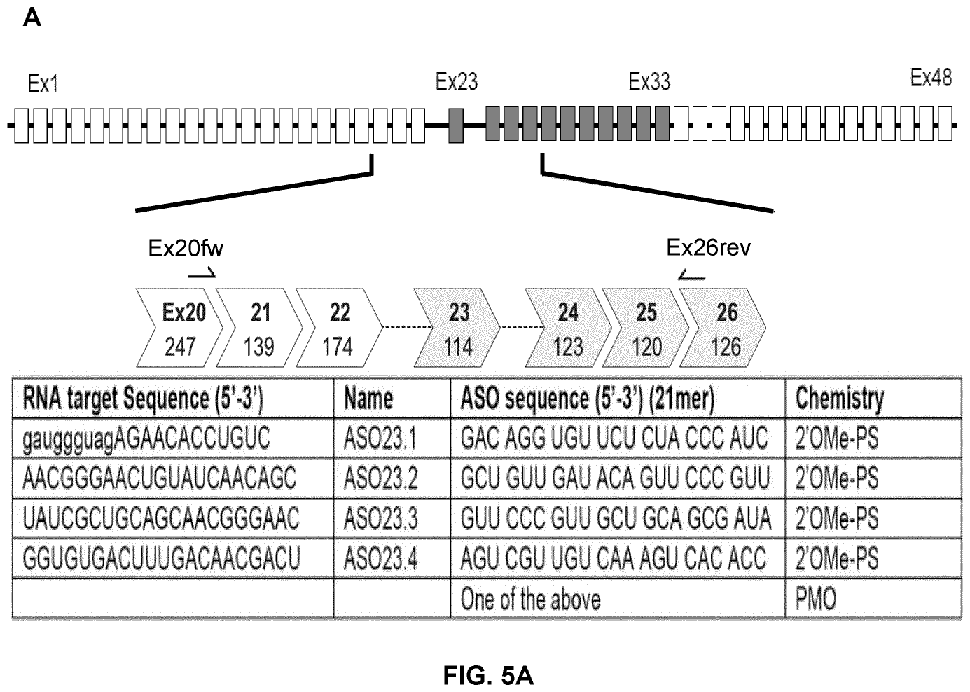

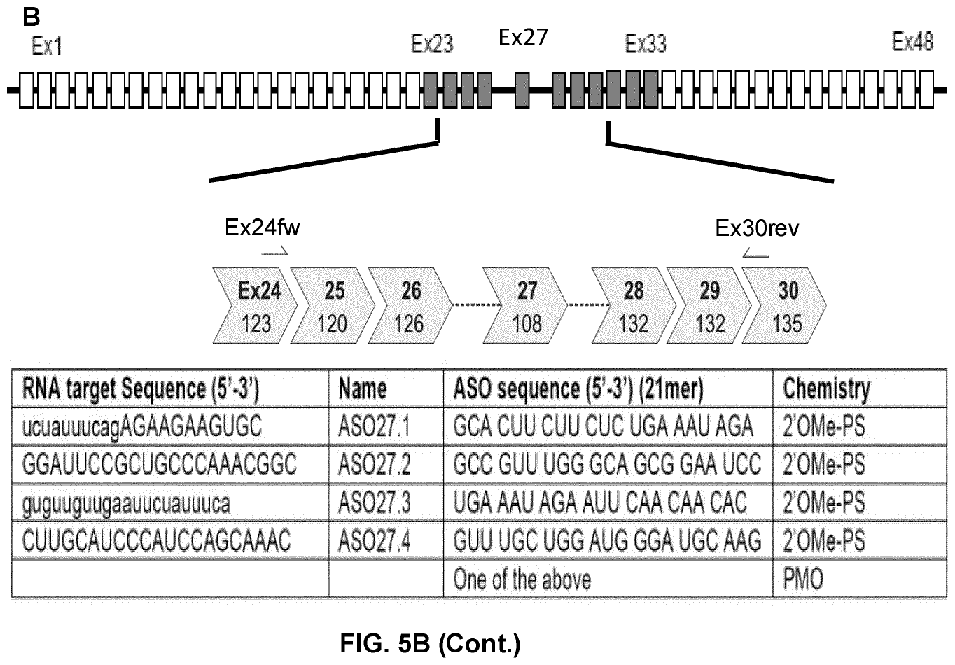

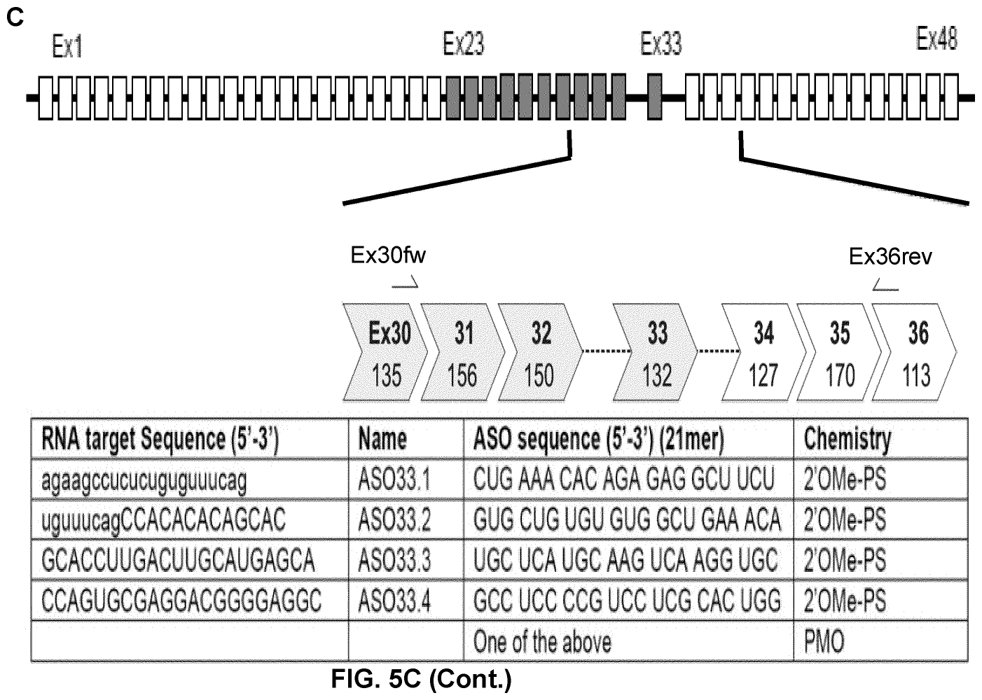

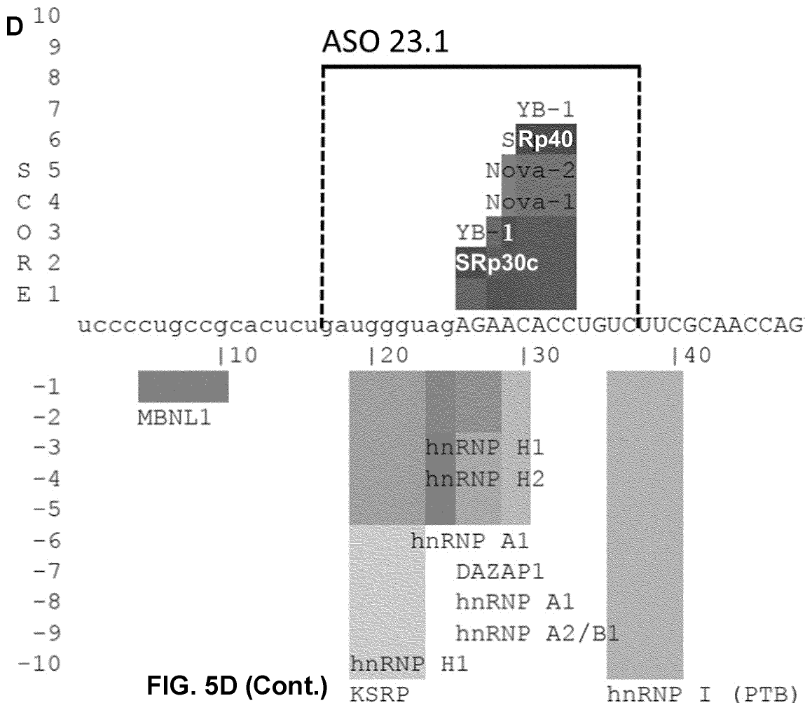

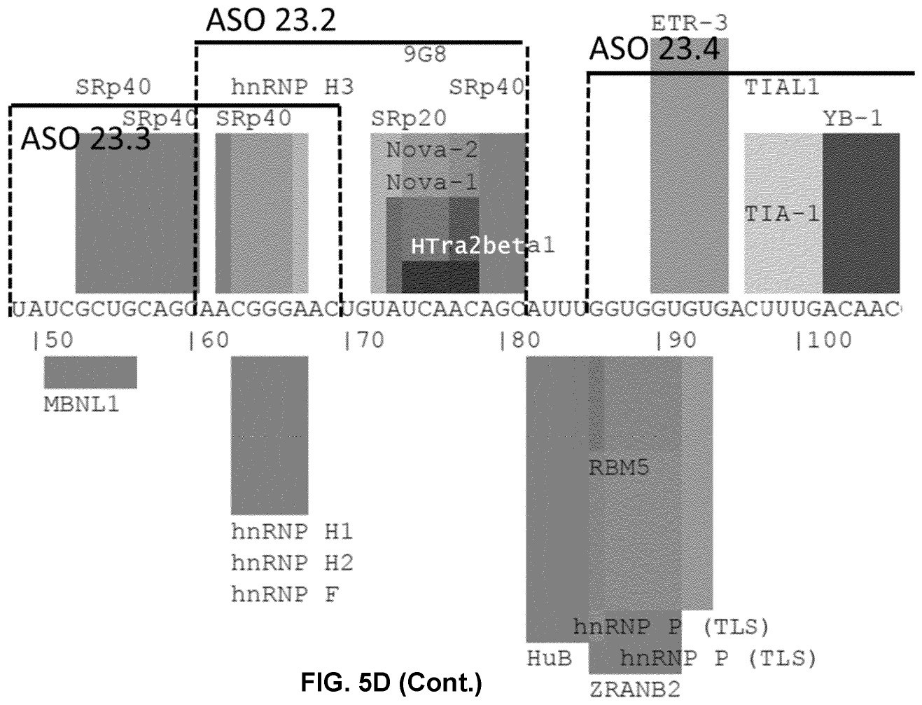

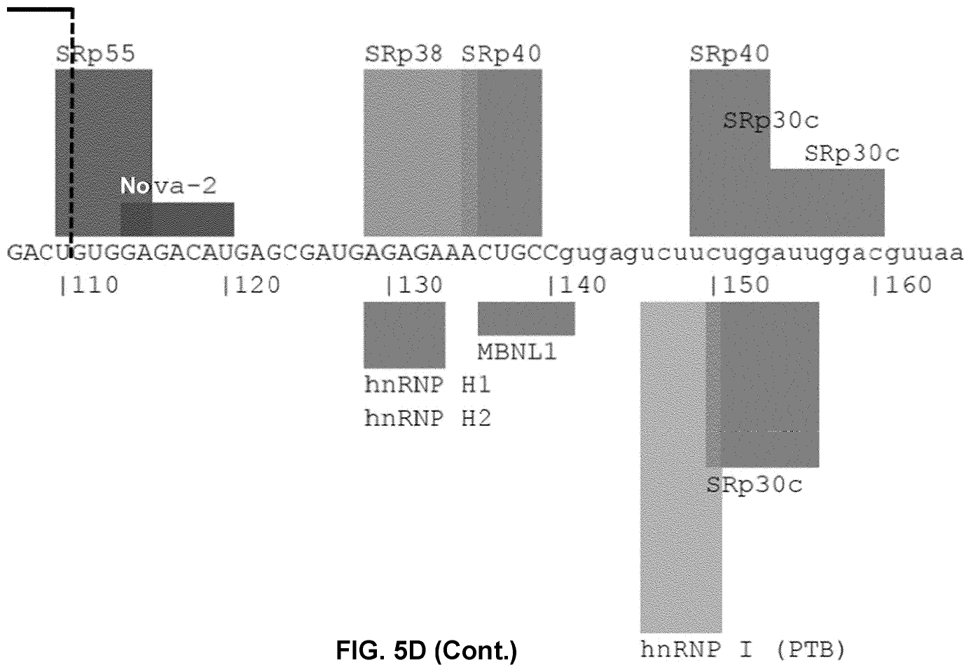

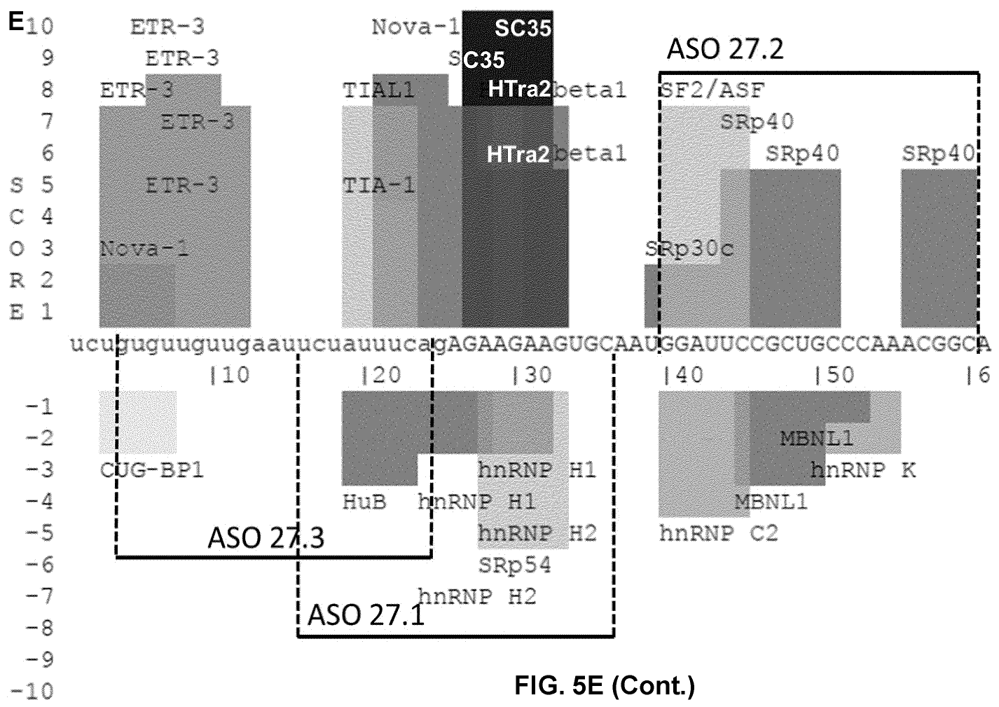

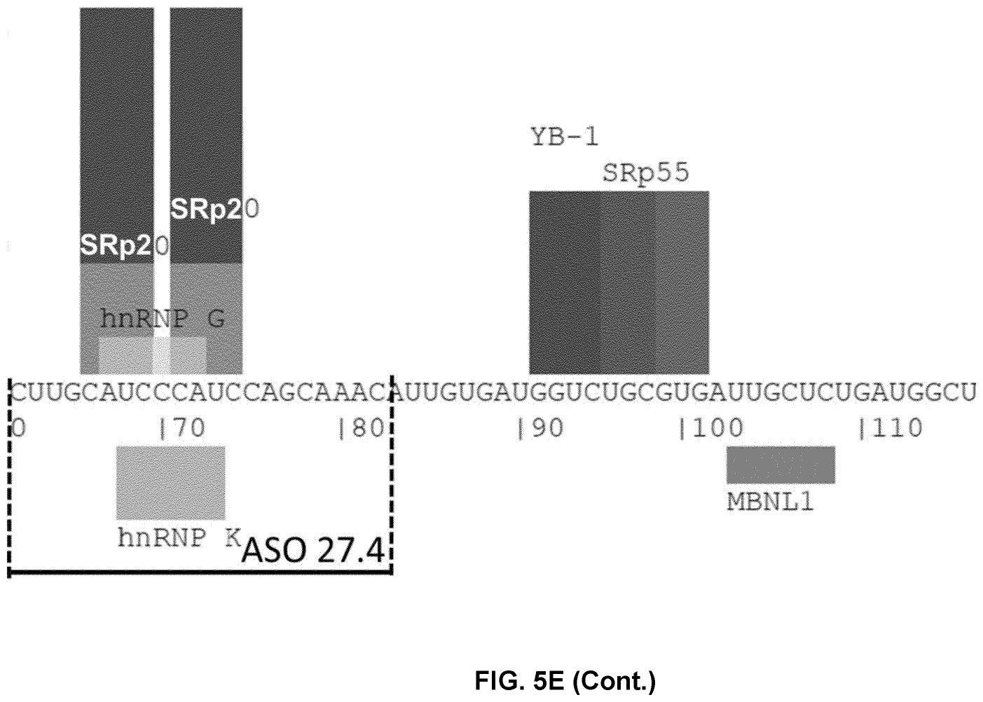

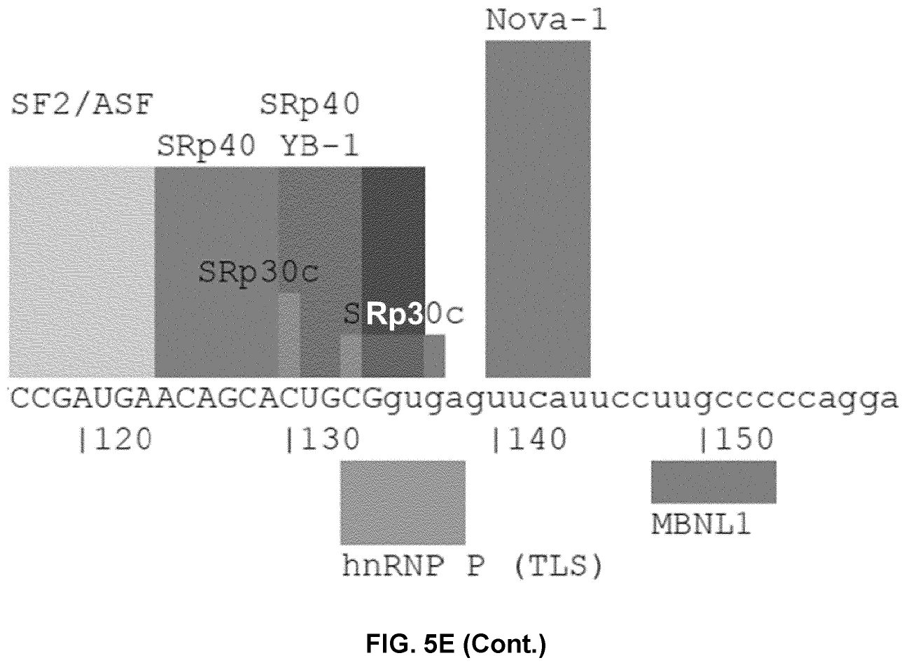

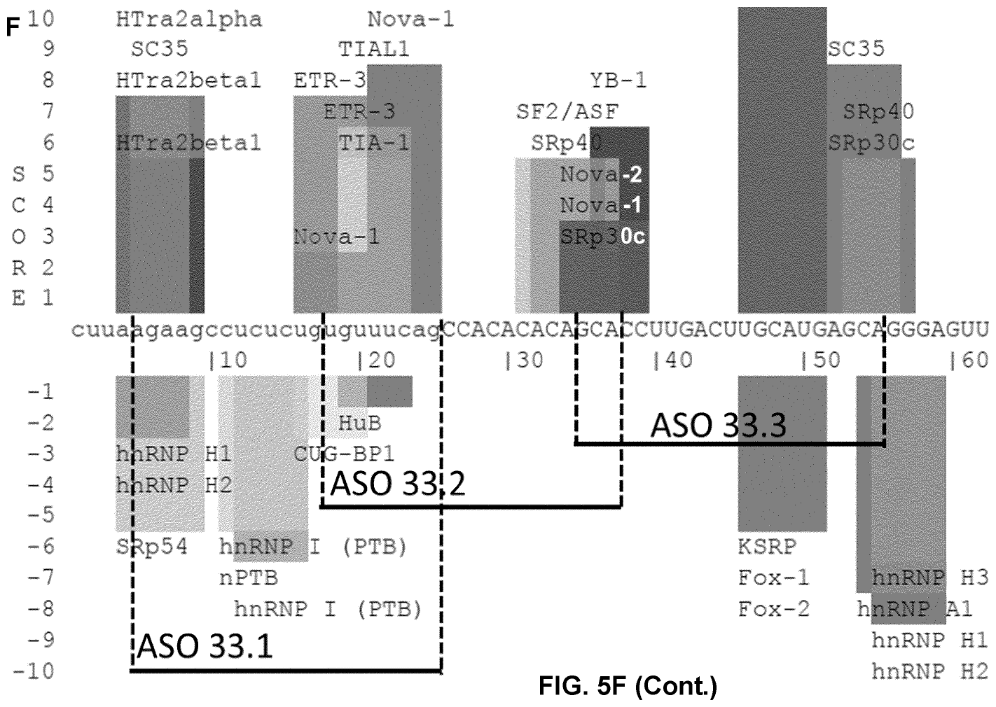

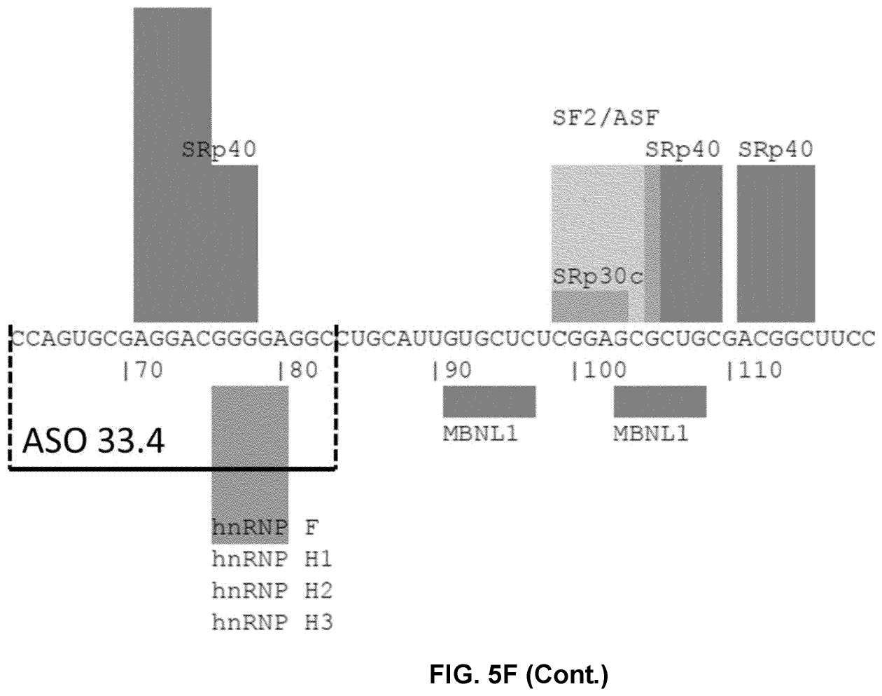

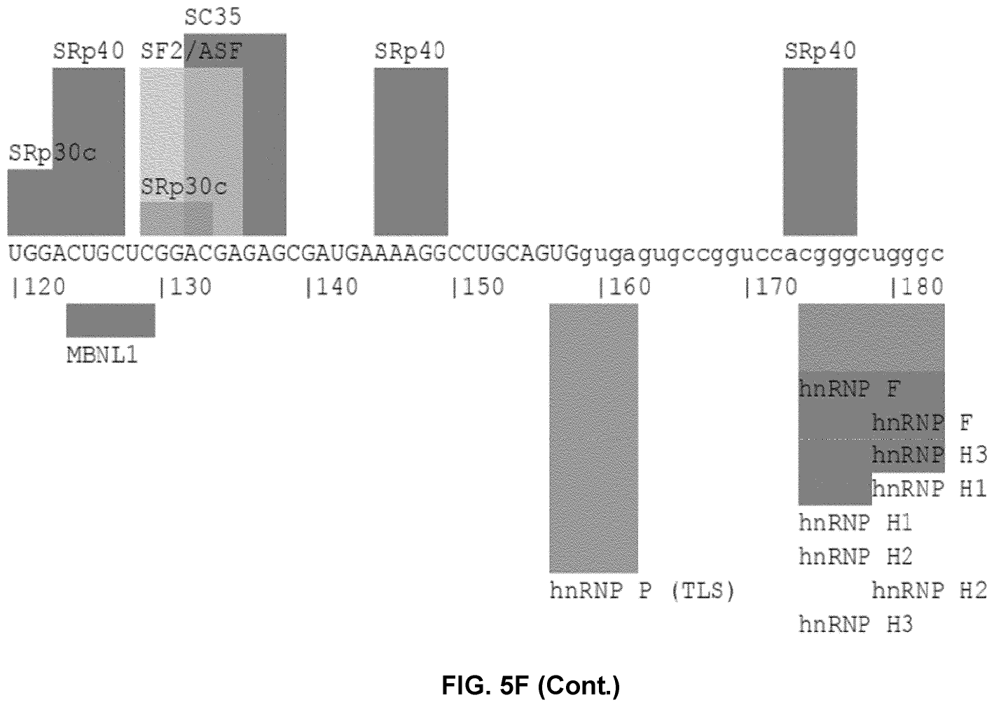

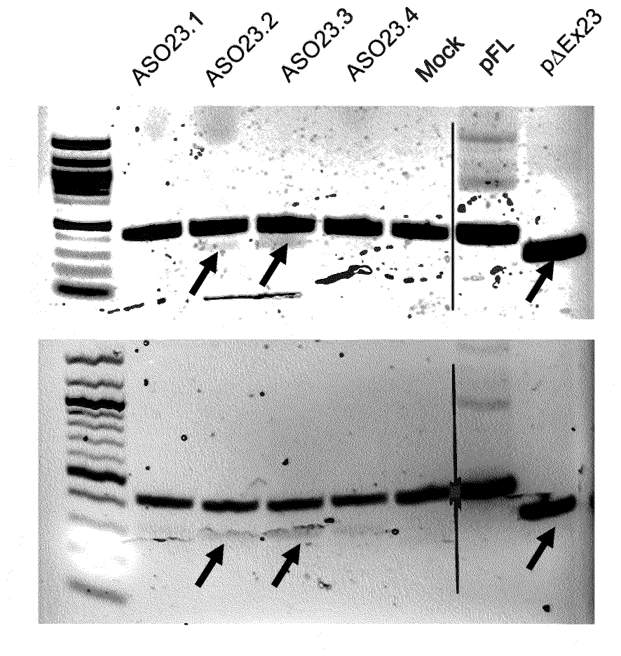

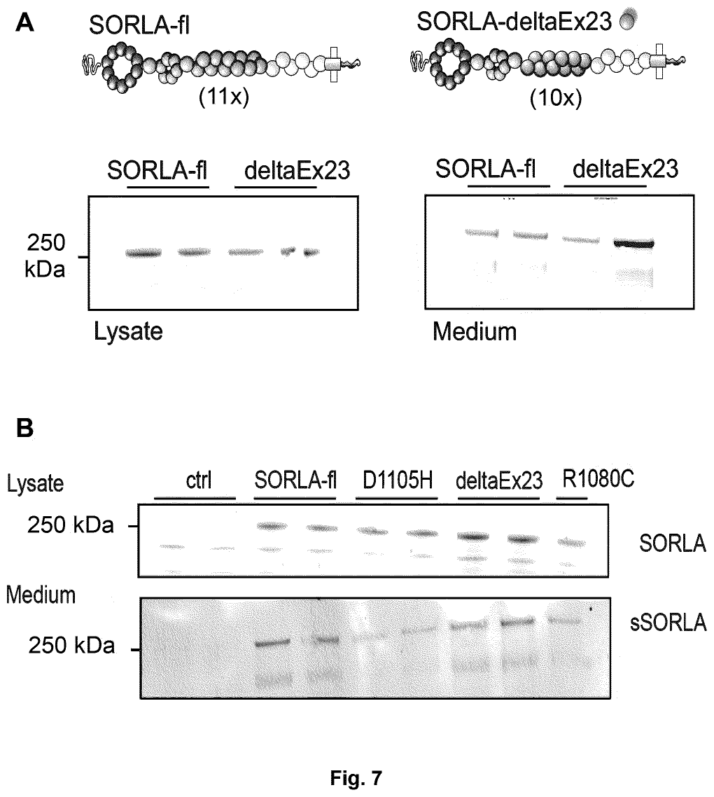

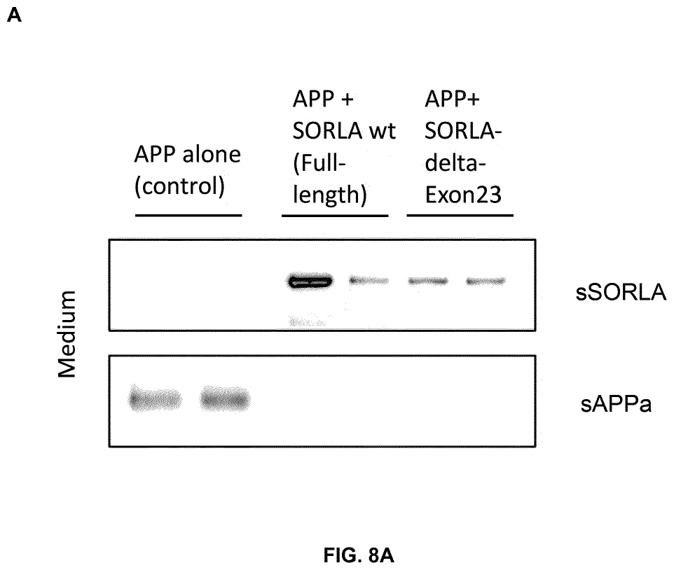

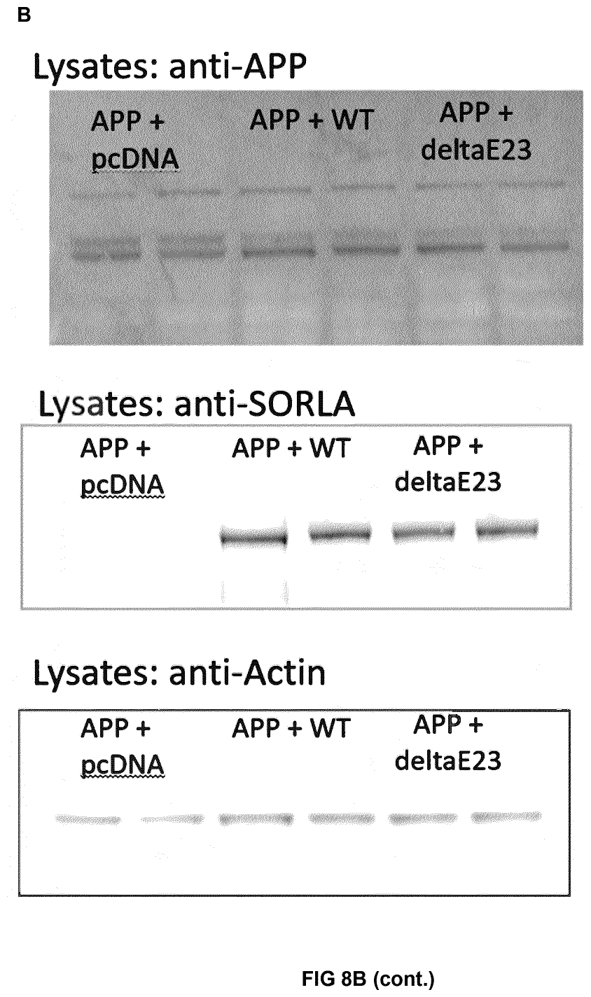

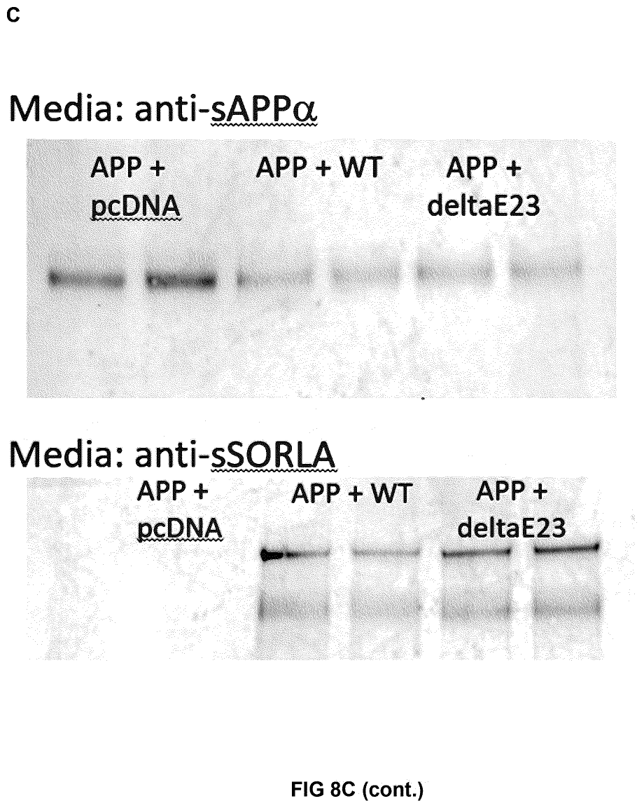

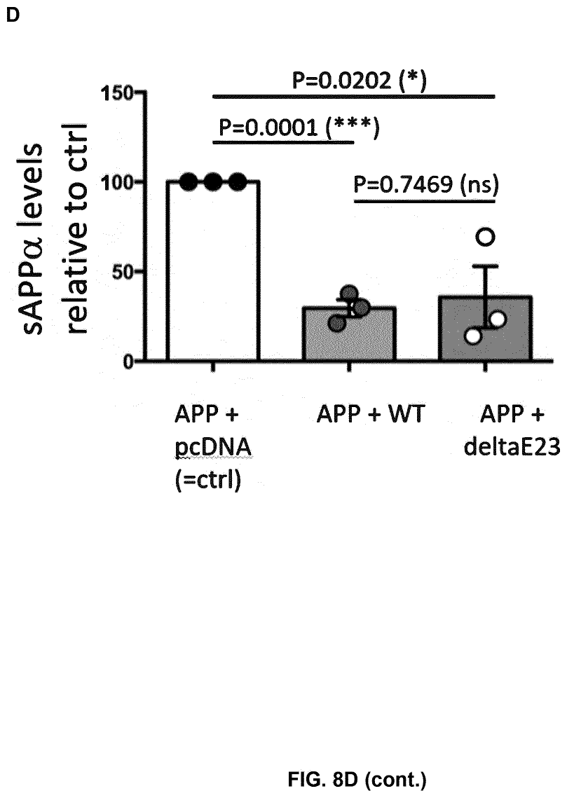

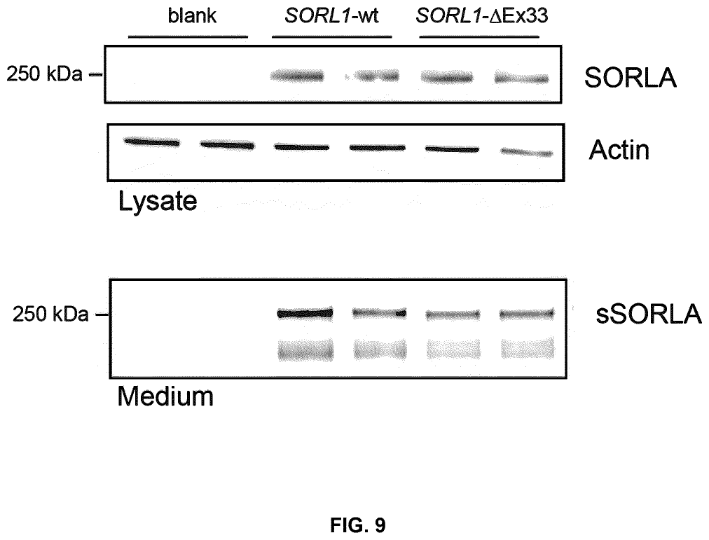

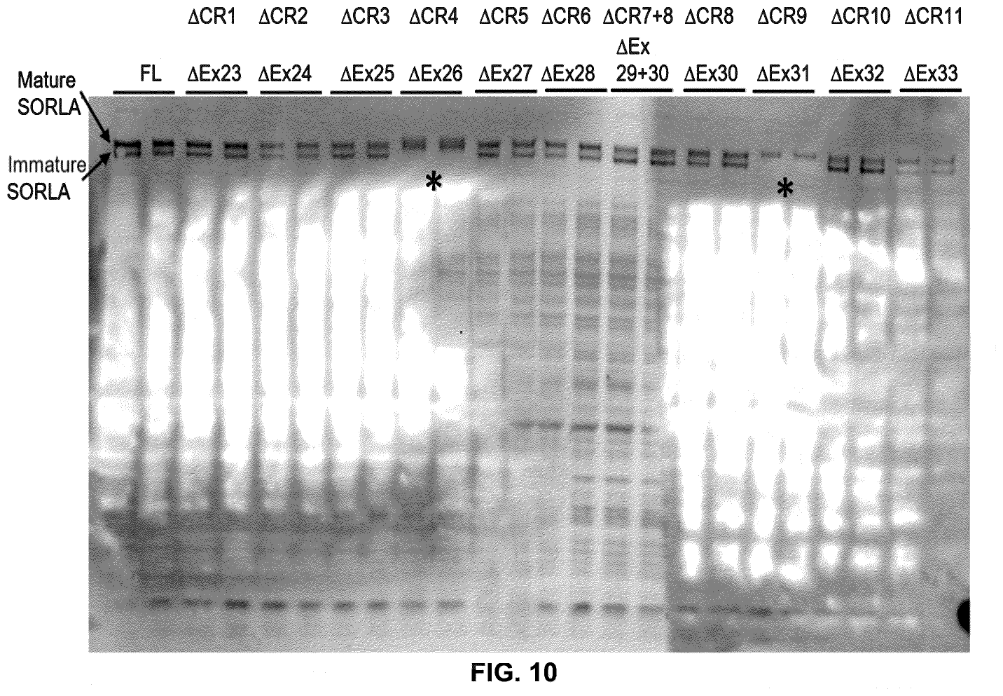

FIG. 4 illustrates the rationale for ASO treatment and the subsequent effects on SORLA activity. FIG. 4A illustrates gene expression of wild type (WT) SORL1, leading to normal protein function and endosomal processing. SORLA expressed from wildtype alleles has eleven CR-domains (11×) and functions in endosomal cargo recycling. FIG. 4B illustrates SORLA expressed from an allele with an AD variant (indicated as black CR domain) of the ONC or CC type leading to receptor misfolding and ER-retention, which potentially also affects the translation product from the wildtype allele. Consequently, mutated SORLA cannot undergo normal endosomal processing, and cannot prevent APP from amyloidogenic processing, thus leading to AD. FIG. 4C illustrates SORLA from disease-alleles treated with exon-skipping ASO, which will contain ten functional CR-domains (10×) and functions indistinguishable from the full-length SORLA protein, i.e. normal protein function and endosomal processing. Consequently, AD symptoms will be reduced, ideally AD will be cured.

### Identification of ASO Candidates Targeting Exon 23, Exon 27 or Exon 33

### Materials and Methods:

Based on predicted ESE (exonic splice enhancer) elements in exons 23, 27, and 33, we identified ASO candidates targeting exon 23 (ASO 23.1, 23.2, 23.3, 23.4 as described below), exon 27 (ASO 27.1, 27.2, 27.3, 27.4) and exon 33 (ASO 33.1, 33.2, 33.3, 33.4 as described below. In a first set of experiments we purchased standard backbone phosphorothioate (PS) ASOs (ASO 23.1, 23.2, 23.3, 23.4) with 2′-O-Methyl (2′OMe) ribose modifications as these molecules are suitable for initial cell experiments and tested these in cell culture experiments. ASOs candidates targeting exons 27 and 33 will be also be purchased and tested in follow-up experiments.

To enhance the skipping efficiency, we will systematically define the optimal sequence, and explore effects of different backbone and ribose chemistry, and we will in additional experiments design a new series of ASOs based on the result from the initial screen selecting the ASO targeting the ESE with the highest effect on exon skipping.

For the ribose chemistry we plan to test effect on 2′-O-methoxyethyl modifications. In particular, we will test ASOs with modified backbone chemistry, including phosphorodiamidate morpholine oligomers (PMO), peptide nucleic acids (PNA), and locked nucleic acids (LNAs) (as outlined in Dhuri 2020).

### Results:

We performed in silico analysis to identify (predicted) strong exonic spice enhancers (ESE), using the online-tool SpliceAid (Piva 2009). Due to the high sequence similarity among SORLA CR-domains (14 of 40 positions contain conserved amino acids among the 11 CR-domains), we decided to prioritize sequences that target the 3′ss boundary. A further advantage of targeting the transcript at the 3′ss is the distance to the sequence that encode residues that are part of the Calcium cage, as an ASO should be able to target exons containing disease-variants that affect the calcium cage—and therefore in the optimal situation not being part of the ASO sequence, which would result in a mismatch for the disease-allele. In further experiments, other options will be explored, such as targeting the, 3′ splice site (3′ss) boundary, the 5′ splice site (5′ss) boundary, and/or one or more exonic splice enhancer (ESE) sites in relation to any one of exons 23 to 33.

For each of the three exons 23, 27, and 33, we selected a total of four sequences, targeting the ESE with the highest positive scores which predict the presence of a splicing enhancing sequence (negative score predicts a splice repressor sequence): ASO23.1, ASO23.2, ASO23.3, ASO23.4 (for Ex23, see FIG. 5A, D), ASO27.1, ASO27.2, ASO27.3, ASO27.4 (for Ex27, see FIG. 5B, E), and ASO33.1, ASO33.2, ASO33.3, and ASO33.4 (Ex33 see FIG. 5C, F) as outlined in FIG. 5 and Tables 1-3).

In FIG. 5D-F, bars in different shades of grey indicate binding sites for splice factors. If an ASO targets these sites, it is likely to induce exon skipping, as the ASO blocks the access for the splice factors to their target sequences, and consequently splicing is hampered and the exon is skipped.

### Exon 23

TABLE 1
ASOs targeting Exon 23

| RNA target | | ASO sequence | |
|---|---|---|---|
| Sequence | | (5′-3′) | |
| (5′-3′) | Name | (21mer) | Chemistry |
| gauggguagAG | ASO23.1 | GACAGGUGUUCU | 2′OMe-PS |
| AACACCUGUC | | CUACCCAUC | |
| AACGGGAACUG | ASO23.2 | GCUGUUGAUACA | 2′OMe-PS |
| UAUCAACAGC | | GUUCCCGUU | |
| UAUCGCUGCAG | ASO23.3 | GUUCCCGUUGCU | 2′OMe-PS |
| CAACGGGAAC | | GCAGCGAUA | |
| GGUGUGACUUU | ASO23.4 | AGUCGUUGUCAA | 2′OMe-PS |
| GACAACGACU | | AGUCACACC | |
| | | One of the | PMO |
| | | above | |

The following sequence (5′-3′, sense strand) shows Exon 23 (capital letters), as well as 5′ and 3′ flanking intron sequences (small letters):

tcccctgccgcactctgatgggtagAGAACACCTGTCTTCGCAACCAGTA
TCGCTGCAGCAACGGGAACTGTATCAACAGCATTTGGTGGTGTGACTTTG
ACAACGACTGTGGAGACATGAGCGATGAGAGAAACTGCCgtgagtcttct
ggattggacgttaa

The respective (partial) precursor mRNA transcript including Exon 23 is (underlined: target sites of ASO23.1-ASO23.4):

uccccugccgcacucugauggguagAGAACACCUGUCUUCGCAACCAGUA
UCGCUGCAGCAACGGGAACUGUAUCAACAGCAUUUGGUGGUGUGACUUUG
ACAACGACUGUGGAGACAUGAGCGAUGAGAGAAACUGCCgugagucuucu
ggauuggacguuaa

### Exon 27

TABLE 2
ASOs targeting Exon 27

| RNA targe | | ASO sequence | |
|---|---|---|---|
| Sequence | | (5′-3′) | |
| (5′-3′) | Name | (21mer) | Chemistry |
| ucuauuucagA | ASO27.1 | GCACUUCUUCUC | 2′OMe-PS |
| GAAGACAGUG | | UGAAAUAGA | |
| GGAUUCCGCUG | ASO27.2 | GCCGUUUGGGCA | 2′OMe-PS |
| CCCAAACGGC | | GCGGAAUCC | |
| guguuguugaa | ASO27.3 | UGAAAUAGAAUU | 2′OMe-PS |
| uucuauuuca | | CAACAACAC | |
| CUUGCAUCCCA | ASO27.4 | GUUUGCUGGAUG | 2′OMe-PS |
| UCCAGCAAAC | | GGAUGCAAG | |
| | | One of the | PMO |
| | | above | |

The following sequence (5′-3′, sense strand) shows Exon 27 (capital letters), as well as 5′ and 3′ flanking intron sequences (small letters):

tctgtgttgttgaattctatttcagAGAAGAAGTGCAATGGATTCCGCTG
CCCAAACGGCACTTGCATCCCATCCAGCAAACATTGTGATGGTCTGCGTG
ATTGCTCTGATGGCTCCGATGAACAGCACTGCGgtgagttcattccttgc
ccccagga

The respective (partial) precursor mRNA transcript including Exon 27 is (underlined: target sites of ASO27.1-ASO27.4):

ucuguguuguugaauucuauuucagAGAAGAAGUGCAAUGGAUUCCGCUG
CCCAAACGGCACUUGCAUCCCAUCCAGCAAACAUUGUGAUGGUCUGCGUG
AUUGCUCUGAUGGCUCCGAUGAACAGCACUGCGgugaguucauuccuugc
ccccagga

### Exon 33

TABLE 3
ASOs targeting Exon 33

| RNA target | | ASO sequence | |
|---|---|---|---|
| Sequence | | (5′-3′) | |
| (5′-3′) | Name | (21mer) | Chemistry |
| agaagccucuc | ASO33.1 | CUGAAACACAGA | 2′OMe-PS |
| uguguuucag | | GAGGCUUCU | |
| uguuucagCCA | ASO33.2 | GUGCUGUGUGUG | 2′OMe-PS |
| CACACAGCAC | | GCUGAAACA | |
| GCACCUUGACU | ASO33.3 | UGCUCAUGCAAG | 2′OMe-PS |
| UGCAUGAGCA | | UCAAGGUGC | |
| CCAGUGCGAGG | ASO33.4 | GCCUCCCCGUCC | 2′OMe-PS |
| ACGGGGAGGC | | UCGCACUGG | |
| | | One of the | PMO |
| | | above | |

The following sequence (5′-3′, sense strand) shows Exon 33 (capital letters), as well as 5′ and 3′ flanking intron sequences (small letters):

cttaagaagcctctctgtgtttcagCCACACACAGCACCTTGACTTGCAT
GAGCAGGGAGTTCCAGTGCGAGGACGGGGAGGCCTGCATTGTGCTCTCGG
AGCGCTGCGACGGCTTCCTGGACTGCTCGGACGAGAGCGATGAAAAGGCC
TGCAGTGgtgagtgccggtccacgggctgggc

The respective (partial) precursor mRNA transcript including Exon 33 is (underlined: target sites of ASO33.1-ASO33.4):

cuuaagaagccucucuguguuucagCCACACACAGCACCUUGACUUGCAU
GAGCAGGGAGUUCCAGUGCGAGGACGGGGAGGCCUGCAUUGUGCUCUCGG
AGCGCUGCGACGGCUUCCUGGACUGCUCGGACGAGAGCGAUGAAAAGGCC
UGCAGUGgugagugccgguccacgggcugggc

To evaluate the effect on the intended exon skipping, we designed a set of primers that span the targeted exons, which allowed amplification of SORL1 transcripts in cells transfected with increasing amounts of the different ASOs (20, 50, 100, and 200 nanomolar). The skipping of an exon leads to a smaller-sized amplicon with 114 bp, 108 bp, or 132 bp less for each of exons 23, 27, and 33, respectively.

The affinity, specificity, efficiency, stability and tolerance of an ASO can be increased by chemical modifications of the structures within the monomers and the backbone. In a final set of experiments, we will therefore repeat the experiment using the most effectful ASO sequence with a number of chemical modifications intended to increase effect on exon skipping. We will also optimize the sequence—where possible without loosing the exon specificity—in order of length (shorter to 18mer or longer to 25mer), and GC-content (aiming for 45-55%), RNA secondary structure (aiming for an optimal melting point), and prediction of dimeric conformations preferred over multimers.

Additionally, Table 4 shows intron-exon boundaries and 3′ splice acceptor or 5′ splice donor sites that can be used to target any one of exons 23 to 24 using ASO's and a procedures as described in this example. Underlined nucleotides represent exons, non-underlined nucleotides represent parts of the neighboring introns. Bold nucleotides represent the nucleotides at the boundary between an intron and an exon, i.e. the ends of an intron (ag/gt).

TABLE 4
Intron-exon boundaries and potential
splice acceptor/donor sites

| | Exon | | | |
|---|---|---|---|---|
| Exon | size | Chromosome | | |
| no. | (bp) | position | 3′ Splice acceptor | 5′ Splice donor |
| 23 | 114 | 121.570.157- | tcccctgccgcactctga | gacatgagcgatgagagaaactg |
| | | 121.570.270 | tgggtagagaacacctgt | ccgtgagtcttctggattggacg |
| | | | cttcgcaaccagta | ttaa |
| 24 | 123 | 121.574.241- | atgttgtctttctatccc | gacaacagtgatgaaagtcattg |
| | | 121.574.363 | attttagctaccaccatc | tggtaaggggacatcacatcctg |
| | | | tgtgacctggacac | aaac |
| 25 | 120 | 121.577.281- | tctctgtttatggtctca | tggtctgatgaagccaactgtac |
| | | 121.577.400 | cctgcagaaatgcaccag | cggtcagtacttcctggactcag |
| | | | tgccggagtgacga | ttga |
| 26 | 126 | 121.583.458- | actgtgtctctcttctct | tccgatgaggatccagtcaactg |
| | | 121.583.583 | gttacagccatctatcac | tggtaaatgcaaattccccagct |
| | | | acctgtgaggcctc | ccct |
| 27 | 108 | 121.586.222- | tctgtgttgttgaattct | gatggctccgatgaacagcactg |
| | | 121.586.329 | atttcagagaagaagtgc | cggtgagttcattccttgccccc |
| | | | aatggattccgctg | agga |
| 28 | 132 | 121.588.020- | cttcccttctctggatcc | gaggatgcggcgtttgcaggatg |
| | | 121.588.151 | cttacagagcccctctgt | ctgtgagttggggcaggcagggg |
| | | | acgcacttcatgga | aggt |
| 29 | 132 | 121.589.259- | tcatggatgtttgttccc | gattattctgatgaagccaactg |
| | | 121.589.390 | tctgtagcccaagatcct | cggtaagatgtccagtctgcctc |
| | | | gagttccacaaggt | ctca |
| 30 | 135 | 121.590.040- | ctgatgatgtggttgatc | tggtctgatgagaaggattgtgg |
| | | 121.590.174 | tctgcagaaaaccccaca | aggtaagaggcccctggggcctg |
| | | | gaagccccaaactg | ggtt |
| 31 | 156 | 121.591.001- | tttccgtgcttggtgttt | gacgaggaagcctgccccttgct |
| | | 121.591.156 | ttctcagattcacatatt | tggtgagttctggcccaggtcct |
| | | | cttcccttctcgac | ctcC |
| 32 | 150 | 121.595.623- | aaattttaaaaatctttt | gatggccgggacgaggccaattg |
| | | 121.595.772 | attttagcaaacgtcact | ccgtgagtagtcagagagccctt |
| | | | gctgcctccactcc | cacc |
| 33 | 132 | 121.604.193- | cttaagaagcctctctgt | gagagcgatgaaaaggcctgcag |
| | | 121.604.324 | gtttcagccacacacagc | tggtgagtgccggtccacgggct |
| | | | accttgacttgcat | gggc |

### ASO Mediated Axon Skipping

### Material and Methods;

A human lymphoblastoid cell line (LCL) with wild-type SORL1 genotype will be transfected with an ASO using standard transfection reagents, e.g. Lipofectamine. Either 24 hrs of 48 hrs after transfection cells will be harvested, and total RNA extracted using RNAeasy isolation kit. Transcripts will be amplified using one-step SuperScript RT with total RNA as template.

To assess exon 23 skipping, SORL1 transcripts will be amplified using Ex20fw (fw=forward) and Ex26rev (rev=reverse) primers and standard PCR conditions. For assessment of exon 27 skipping, SORL1 transcripts will be amplified using Ex24fw and Ex30rev primer pair and for assessment of exon 33 skipping, SORL1 transcripts will be amplified using Ex30fw and Ex36rev primers. The PCR amplicons will be fractionated on 2% agarose gels in Tris-Acetate-EDTA buffer. Relative exon skipping efficiency will be estimated through densitometric analysis of images using ImageJ imaging software.

In another experiment, human HEK293 cells will be transfected with the ASO and 24 hours after transfection, cells and medium will be harvested.

In another experiment iPSC-derived neurons will be used for transfection, and RT-PCR will be done as described above.

In case of the above described ASO candidates targeting exon 23, exon skipping has been tested in vitro. For ASO treatment, HEK cells were seeded on 4-well dish at a density of 1×105 cells per well. The following day, cells were transfected with ASO targeting exon 23 splicing at a final concentration of 250 nM, using Oligofectamine Transfection Reagent (Thermo Fischer, #12255011) diluted in serum-free medium according to manufacturer's protocol. Cells were harvested 48 hours after transfection, followed by RNA extraction using the RNeasy Kit according to manufacturer's protocol (Qiagen), and cDNA was then prepared with the High-Capacity RNA to cDNA kit (Applied Biosystems, #4387406). Exon 23 skipping was verified by RT-PCR using the following primers: Forward, 5′-ACACTGGAAGCAATGCCTGT-3′ (SEQ ID NO: 47); Reverse: 5′-CGGCACTGGTGCATTTCAC-3′ (SEQ ID NO: 48).

### Results:

We tested 4 ASOs (ASO23.1, ASO23.2, ASO23.3, and ASO23.4) directed against exon splice enhancer (ESE) elements of SORL1 exon 23, by transfection of HEK293 cells, harvesting endogenous SORL1 mRNA and did RT-PCR using a primer pair spanning the region around exon 23. PCR product were separated by agarose gel electrophoresis, and data from two independent experiments are shown (FIG. 6, upper and lower figure). In both experiments we observed clear evidence that ASO23.2 and ASO23.3 induced skipping of exon 23, as demonstrated by the presence of a shorter PCR-product (see arrow) that migrates identical to products generated using a plasmid encoding the exon 23-deleted fragment (pΔEx23, a recombinantly produced variant lacking exon 23 used as control) as template. pFL served as control template to identify PCR-products corresponding to fragments that have exon 23 included. Potentially, also ASO 23.1 and ASO 23.4 have a certain exon skipping efficiency, as faint bands of a shorter PCR product are visible as well.

### Conclusion:

Our results show that ASO-mediated exon-skipping can be achieved, as exemplified by the skipping of exon 23 by at least two of our candidate ASOS. We will further identify ASOs that can induce skipping of exon 27 or exon 33 of the human SORL1 transcript, as well as of other exons encoding CR domains. This approach can be used to target and remove any one, or several, of exons 23 to 33.

### Example 2: Retained Functionality of the Modified SORLA Protein (Lacking One or More CR-Domains)

### Aim:

The aim is to determine if a CR-domain, exemplified by CR-domain 1 (corresponding to omitted exon 23), is dispensable for SORLA.

### Material and Methods:

Using specific primers and an expression construct for full-length SORL1, fragments that lead to deletion of exon 23 was generated by PCR. These fragments were joined using Gibson assembly.

N2a cells were transfected with plasmids encoding either the full-length or exon 23-deleted SORLA protein. Western blot of lysates and conditioned medium from transfected cells were performed as described. Note that in N2A cells SORLA shows as one band at around 250 kDa in Western Blots, in contrast to the characteristic double band in HEK cells.

Generation of SORL1 cDNA Constructs

Deletion of individual exons in SORL1 cDNA was performed by Gibson Assembly technology using SORL1-wt cDNA in pcDNA 3.1/zeo vector as template (Jacobsen et al. 2001). Vector and SORL1 fragments were amplified using Herculase II Fusion DNA Polymerase (Agilent) with specific primer sets (Table 5). PCR products were first digested with DpnI enzyme (New England Biolabs) to remove the methylated DNA template, followed by purification using the PCR purification kit (Qiagen, #28104). Purified fragments were ligated using Gibson Assembly kit (NEB, #E5510S) and NEB 5α competent E. coli were transformed with the ligation products according to manufacturer's protocol (NEB, #C29871). Correct deletion of exons in each plasmid was verified by Sanger sequencing (Eurofins).

TABLE 5
PCR primers used for amplification of SORL1
fragments (unless otherwise indicated)

| | SORL1 | Forward primer | Reverse primer |
|---|---|---|---|
| Construct | fragments | (5′-3′) | (5′-3′) |
| SORL1- | E1-22 | AGTAGCGTTCGCCCGAACA | GGTCACAGATGGTGGTAGCTTCT |
| ΔE23 | | TGGCGACACGGAGCAGC | TTGACACAGGTATTGTTCTTGAG |
| | | | C |
| | E24-48 | TACCTGTGTCAAAGAAGCT | CTAGTGAGGAAAGCTCTTTCAGG |
| | | ACCACCATCTGTGACC | CTATCACCATGGG |
| SORL1- | E1-23 | AGTAGCGTTCGCCCGAACA | TCCGGCACTGGTGCATTTGGCAG |
| ΔE24 | | TGGCGACACGGAGCAGC | TTTCTCTCATCGCTCATG |
| | E25-48 | CGATGAGAGAAACTGCCAA | CTAGTGAGGAAAGCTCTTTCAGG |
| | | ATGCACCAGTGCCGGAG | CTATCACCATGGG |
| SORL1- | E1-24 | AGTAGCGTTCGCCCGAACA | CACAGGTGTGATAGATGGCACAA |
| ΔE25 | | TGGCGACACGGAGCAGC | TGACTTTCATCACTGTTGTCTCC |
| | E26-48 | TGATGAAAGTCATTGTGCC | CTAGTGAGGAAAGCTCTTTCAGG |
| | | ATCTATCACACCTGTG | CTATCACCATGGG |
| SORL1- | E1-25 | AGTAGCGTTCGCCCGAACA | ATCCATTGCACTTCTTCTCGGTA |
| ΔE26 | | TGGCGACACGGAGCAGC | CAGTTGGCTTCATCAGAC |
| | E27-48 | TGAAGCCAACTGTACCGAG | CTAGTGAGGAAAGCTCTTTCAGG |
| | | AAGAAGTGCAATGGATTC | CTATCACCATGGG |
| SORL1- | E1-26 | AGTAGCGTTCGCCCGAACA | AGTGCGTACAGAGGGGCTCACAG |
| ΔE27 | | TGGCGACACGGAGCAGC | TTGACTGGATCCTCATCG |
| | E28-48 | GGATCCAGTCAACTGTGAG | CTAGTGAGGAAAGCTCTTTCAGG |
| | | CCCCTCTGTACGCACTTC | CTATCACCATGGGG |
| SORL1- | E1-27 | AGTAGCGTTCGCCCGAACA | GGAACTCAGGATCTTGGGCGCAG |
| ΔE28 | | TGGCGACACGGAGCAGC | TGCTGTTCATCGG |
| | E29-48 | CGATGAACAGCACTGCGCC | CTAGTGAGGAAAGCTCTTTCAGG |
| | | CAAGATCCTGAGTTCC | CTATCACCATGGG |
| SORL1- | E1-28 | GCAGTAGCGTTCGCCCGAA | GAAGGGAAGAATATGTGAATAGC |
| ΔE29Ε30 | | CATGGCGACACGGAGCAGC | ATCCTGCAAACGCCG |
| | E31-48 | TGCGGCGTTTGCAGGATGC | TTCTAGTGAGGAAAGCTCTTTCA |
| | | TATTCACATATTCTTCCCT | GGCTATCACCATGGG |
| | | TCTC | |
| SORL1- | E1-29 | AGTAGCGTTCGCCCGAACA | AGGGAAGAATATGTGAATCGCAG |
| ΔE30 | | TGGCGACACGGAGCAGC | TTGGCTTCATCAGAATAATCG |
| | E31-48 | TGATGAAGCCAACTGCGAT | TAGTGAGGAAAGCTCTTTCAGGC |
| | | TCACATATTCTTCCCTTCT | TATCACCATGGG |
| | | C | |
| SORL1- | E1-30 | AGTAGCGTTCGCCCGAACA | AGGCAGCAGTGACGTTTGCTCCA |
| ΔE31 | | TGGCGACACGGAGCAGC | CAATCCTTCTCATCAGACC |
| | E32-48 | TGAGAAGGATTGTGGAGCA | TAGTGAGGAAAGCTCTTTCAGGC |
| | | AACGTCACTGCTGCCTC | TATCACCATGGG |
| SORL1- | E1-31 | AGTAGCGTTCGCCCGAACA | TCAAGGTGCTGTGTGTGGCAAGC |
| ΔE32 | | TGGCGACACGGAGCAGC | AAGGGGCAGGCTTC |
| | E33-48 | AGCCTGCCCCTTGCTTGCC | TAGTGAGGAAAGCTCTTTCAGGC |
| | | ACACACAGCACCTTGAC | TATCACCATGGG |
| SORL1- | E1-32 | AGTAGCGTTCGCCCGAACA | TGTACACAGTCAACTCATGGCAA |
| ΔE33 | | TGGCGACACGGAGCAGC | TTGGCCTCGTCCC |
| | E34-48 | GGACGAGGCCAATTGCCAT | TAGTGAGGAAAGCTCTTTCAGGC |
| | | GAGTTGACTGTGTACAAAG | TATCACCATGGG |

Transfection of Cells with SORL1 Exon Deletion Constructs

For APP processing and SORLA shedding analyses, N2a cells were cultivated in DMEM supplemented with 10% FBS and penicillin/streptomycin, and the day before transfection 5×10⁵ cells per well were seeded in a 6-well plate. Cells were then transiently co-transfected with a myc-flagged construct encoding APP and either SORL1-wt or exon deletion constructs using Fugene HD Transfection Reagent according to manufacturer's protocol (Promega). After 48 hs recovery, culture medium was changed to conditional serum-free medium, and collection of lysates and media was performed after further 48 hs.

### Western Blotting

Equal amount of proteins from lysates and media of N2a transfected cells were loaded on Nupage 4-12% Bis-Tris gels (Invitrogen, #NP0321BOX) and transferred onto nitrocellulose membranes using iBlot 2 Gel Transfer Device (Life Technologies). Membranes were probed over night at 4° C. with the following primary antibodies anti-myc (1:1000, Invitrogen), anti-APP (WO2, 1:1000, Sigma, MABN10), LR11 (1:500, BD Transduction Laboratories), anti-solSORLA (1:1000, IgG 5387, Jacobsen et al. 2001), and anti-actin (1:5000, Sigma, A2066). The following day, membranes were washed and incubated with HRP-conjugated secondary antibodies (anti-mouse, anti-rabbit; 1:1500) for 1 hr at room temperature. Proteins were detected with SuperSignal West Femto Maximum Sensitivity Substrate (Thermo Fischer Scientific) using the iBright Imaging system (Thermo Fischer).

### Results:

We prepared a cDNA encoding human SORLA deleted of exon 23 encoding the first CR-domain of the SORLA CR-cluster. Transfected cells showed indistinguishable expression level in lysates (FIG. 7A, left panel). The amount of shed SORLA (sSORLA) from the cell surface into the medium was indistinguishable for cells transfected with SORLA-wt or with SORLA-delta-Exon23 (FIG. 7A, right panel), consequently the SORLA variant lacking the CR domain corresponding to exon23 seems to undergoes similar trafficking and maturation as wt SORLA, and is functional.

This is due to the fact that, to be able to undergo endosomal trafficking and maturation, SORLA needs to interact with its respective ligands. Since shed SORLA lacking the CR domain corresponding to exon 23 can be detected at similar levels than full length SORLA, this interaction seems to be functional

### Conclusion:

We conclude that SORLA deleted of its first CR-domain can be expressed and sorted within the cell in a manner indistinguishable for wild-type SORLA.

### Example 3: Retained Functionality of SORLA Protein Lacking One CR-Domain (Corresponding to Skipped Exon 23) Compared to Mutant Protein Corresponding to Pathogenic SORL1 Variants Locating to the Same CR-Domain

### Aim:

The aim is to compare activity for CR-mutant protein versus CR-deleted protein exemplified by the most N-terminal CR-domain or SORLA.

### Material and Methods:

N2a cells were transfected with plasmids encoding either the full-length SORLA, full-length SORLA with mutation D1105H, full-length SORLA with mutation R1080C, or SORLA deleted of CR1 (by removal of the sequence encoded by exon 23). Western blot analyses of lysates and conditioned medium from transfected cells were performed with the commercially available antibody LR11 (anti-SORLA antibody, e.g. Sigma Aldrich, SAB2500979).

Further details regarding material and methods used are described in Example 2.

### Results:

We prepared a cDNA encoding human SORLA deleted of exon 23 encoding the first CR-domain of the SORLA CR-cluster. Using standard site-directed mutagenesis we prepared mutant constructs corresponding to variants D1105H or R1080C. The variant R1080C corresponds to the group of identified variants with odd-numbered cysteines and considered pathogenic. The variant D1105H affects a residue important to form an Asx-turn in CR-domains, and also considered strongly pathogenic to an extend similar as variants that affect the Calcium-cage.

Transfected cells showed indistinguishable expression level in lysates (FIG. 7B, upper panel). The amount of shed SORLA (sSORLA) from the cell surface into the medium was indistinguishable for cells transfected with SORLA-wt or with SORLA-delta-Exon23, whereas it was strongly reduced in cells that express pathogenic SORL1 variants (as here represented by mutations D1105H and R1080C) (FIG. 7B, lower panel).

### Conclusion:

We conclude that SORLA with pathogenic variants in CR1 (exon 23) show strongly reduced shedding and thus production of sSORLA, whereas SORLA deleted of its first CR-domain is shed in a manner indistinguishable for wild-type SORLA.

We further conclude that the production of sSORLA is compromised in cells that express pathogenic SORL1 variants, and that monitoring sSORLA is a very efficient method for evaluation of ASO-induced exon skipping efficiency. This further implies that patients that have been treated with an ASO to correct SORL1 variants would be accompanied by an increase of their cerebrospinal fluid (CSF) sSORLA, since only the SORLA variant lacking the CR domain corresponding to the mutated exon would get shed, while full length SORLA with a CR domain mutation is likely to get retained in the ER and is not able to undergo endosomal processing. This data supports the herein disclosed exon skipping strategy to cure SORL1-associated AD.

### Example 4: Indications for AD Therapeutic Effect of ASO-Mediated Exon-Skipping of Exon 23

### Aim:

The aim is to demonstrate that SORLA deleted of the CR-domain encoded by exon 23 is able to protect APP from (endosomal) processing.

### Material and Methods:

N2a cells were transfected with APP alone (control) or with APP in combination with either SORLA-wt (full length), or SORLA-delta-Exon23 (deletion of exon 23 by a cDNA cloning strategy. Lysates and conditioned medium from cells were analysed by Western blotting using antibodies for APP, SORLA or Actin (in lysate samples) or shed APPα (sAPPα) or shed SORLA (sSORLA) (in medium samples).

Further details regarding material and methods used are described in Example 2.

### Results:

Experiment 1: In this setting, N2a cells express no or negligible amounts of endogenous SORLA, i.e. in Western Blot endogenous SORLA cannot be detected compared to exogenous SORLA (overexpression via transfection). Lysates (data not shown) and conditioned medium from the transfected cells were analysed by Western blotting using antibodies for the SORLA ectodomain (5387) or for APP (anti-myc for cellular form or WO2 for shed sAPPa).

Cells with no exogenous SORLA overexpression (control) show a strong signal for sAPP in the medium. In contrast, medium from cells transfected with either full-length SORLA or the SORLA construct with deletion of exon 23 show strongly reduced levels of sAPP (FIG. 8A).

### Experiment 2:

Again, we observed that the level of shed SORLA (sSORLA) is similar between SORLA-WT and SORLA-ΔEx23, showing that deletion of CR1 (encoded by Exon 23) has no observable impact on receptor biology.

We also again observed that the two SORLA variants (full-length and SORLA-delta-Exon23) protein have indistinguishable effect on lowering sAPPα production by decreasing APP proteolysis (FIG. 8B-C).

Blots from three independent replicates were quantified, and data presented as means of duplicate samples with levels relative to cells with no exogenous SORLA (FIG. 8D). The quantification further shows that SORLA ΔEx23 is as effective as WT to decrease shed APPa (sAPPa).

### Conclusion:

SORLA deleted of CR1 (encoded by exon 23) is as efficient in reducing APP processing as is the full-length SORLA receptor. This demonstrates that targeted deletion of CR1 by an ASO to induce skipping of a mutated exon 23 will lead to a functional receptor.

### Example 5: Retained Functionality of the Modified SORLA Protein Lacking CR-Domain 11

### Aim:

The aim is to determine if a CR-domain, exemplified by CR-domain 11 (corresponding to omitted exon 33), is dispensable for SORLA.

### Material and Methods:

N2a cells were transfected with constructs for either SORL1-WT or SORL1-ΔEx33 and lysates and conditioned medium from cells were analysed by Western blotting using antibodies for SORLA or Actin (in lysate samples) or shed SORLA (sSORLA) (in medium samples).

Further details regarding material and methods used are described in Example 2.

### Results:

We observed that the level of shed SORLA (sSORLA) is similar between SORLA-WT and SORLA-ΔEx33, showing that deletion of CR11 (encoded by Exon 33) has no observable impact on SORLA receptor biology, i.e. even SORLA lacking CR11 is processed and shed similarly to SORLA-WT (FIG. 9; medium samples).

### Conclusions:

We conclude that SORLA deleted of CR-domain 11 can be expressed and sorted within the cell in a manner indistinguishable for wild-type SORLA. Our data indicates that the deletion of a certain CR-domain which might comprise a pathologic mutation, here exemplified by CR11, does not compromise SORLA processing and function, and as such exon skipping of the respective CR domain is a feasible treatment strategy.

### Example 6: Expanding Technology to Additional CR-Domain Exons

### Aim:

We wished to determine which SORL1 exons are suitable for our ASO-induced exon skipping therapeutic strategy.

### Material and Methods:

Examples 3 to 5 indicate that certain SORLA exons may be skipped while still retaining SORLA function (e.g. cellular processing, shedding, impact on APP processing). We went on to investigate the deletion of further exons and performed a systematic deletion of Exon 23-33 (recombinantly produced constructs).

We generated plasmids encoding SORLA deleted of single CR-domains (except CR7 that was included in a tandem deletion for CRF7+8; ΔEx29+30).

HEK293 cells were transfected with constructs engineered to generate SORLA proteins deleted for individual CR-domains, i.e. CR1 (ΔEx23), CR2 (ΔEx24), CR3 (ΔEx25), CR4 (ΔEx26), CR5 (ΔEx27), CR6 (ΔEx28), CR7+8 (ΔEx29+30), CR8 (ΔEx30), CR9 (ΔE31), CR10 (ΔE32) or CR11 (ΔE33). Lysates were prepared from cells harvested 72 hours post-transfection, proteins separated by 26-lane SDS-PAGE NuPAGE system, and analysed by Western blotting analysis with a polyclonal SORLA serum from rabbit raised against the SORLA extracellular fragment (sol-SORLA).

Further details regarding material and methods used are described in Example 2.

### Results:

The western blot of FIG. 10 shows the results of systematic deletion of each of exons 23-33 corresponding to SORLA CR domains 1-11. SORLA blots are known to show double bands when expressing SORLA in HEK cells, with an upper band (running slower in the gel due to a larger molecular size) representing mature SORLA, and a lower band (running faster in the gel due to a smaller molecular size). This can, for example, be seen in the double band indicated for full-length (FL) SORLA in lanes 1 and 2.

Each of the construct led to expression of a specific, CR-domain-deleted SORLA receptor. Interestingly, some deletions showed a surprising result suggesting that potentially not all CR-domains can be deleted without disturbance of receptor function, i.e. CR4 (ΔE26) and CR9 (ΔEx31) where the SORLA double band pattern is disturbed (*). We will analyse this further in follow-up experiments. Similar results were obtained when the experiment was repeated. Challenges regarding deletion of exon 29 are discussed below in Example 7. Skipping of exon 29 generates an unintended stop-codon in the novel exon-exon boundary between exon 28 and exon 30. Accordingly, targeting of variants in exon 29 needs concomitant deletion of a neighbouring CR-domain.

### Conclusion:

We conclude that several exons of SORL1 are suitable for the herein disclosed ASO-induced exon-skipping therapy against AD for carriers of pathogenic SORL1 variants in these exons. Our results further indicate that certain CR domains may be more suitable for deletion, e.g. via exon skipping, as others.

### Example 7: Challenge for Deletion of Exon 29: Double-Deleting ASO Treatment

### Aim:

Skipping of exon 29 generates an unintended stop-codon in the novel exon-exon boundary between exon 28 and exon 30. Accordingly, targeting of variants in exon 29 needs concomitant deletion of a neighbouring CR-domain.

### Material and Methods:

We will establish exon skipping of SORLA exon 29 and a neighbouring CR-domain, for example exon 28 or exon 30. To delete 2 exons, two ASOs targeting the respective target sites for exon 28 and exon 29, or exon 29 and exon 30, will be needed. Further details regarding material and methods used are described in Example 2.

### Results:

We will prepare cDNA for SORLA deleted of exons 28+29 and for SORLA deleted of exons 29+30.

### Conclusion:

Feasibility of ASO mediated exon-skipping of more than one exon will be shown.

### Example 8: ASO Rescue Experiment

### Aim:

We will provide evidence that an ASO targeting a mutated SORL1 exon 23/27/33 will increase activity of the treated allele.

### Material and Methods:

A patient-derived cell line (i.e. lymphoblast) will be immortalized using EBV. In parallel control cells from healthy carriers will be obtained and immortalized in parallel. Cells of both origins will be treated with ASO for 48 hrs or left untreated for control. Cell lysates and conditioned medium will be analysed using Western Blot and an antibody for SORLA.

### Results:

Untreated cells from healthy carriers will show two clear bands in lysates corresponding to mature and immature full-length SORLA, whereas untreated cells from patients with a SORLA CR-domain pathogenic variant will predominantly display only the immature receptor variant.

Cells treated with the ASO will both show strong signals for the mature protein in lysates. WB analysis of the medium samples will show a similar pattern with high levels of sSORLA from cells having mature intracellular SORLA protein.

### Conclusion:

We will conclude that an ASO can correct the production of a pathogenic, misfolded SORLA protein (with 11 CR-domains of which one is pathologically mutated), and ASO treatment will lead to skipping of the exon harbouring the pathogenic variant, and these cells express functional protein (with 10 CR-domains omitting the mutated CR domain) as evidenced by the presence of mature SORLA in lysates and the presence of sSORLA in the medium.

### Example 9: Optimization of ASO23.2 and ASO23.3

### Aim:

The aim is to optimize lead ASOs targeting SORL1 Exon 23, for example based on the results as described in Example 1.

### Material and Methods:

Based on our described observations that ASO23.2 and ASO23.3 showed the most efficient exclusion of SORL1 exon 23 in our preliminary studies using transfected HEK293 cells (Example 1), we will prepare new variants of these two ASOs aiming to optimize their efficiency.

First, we will trim the sequence by moving one-base-at-the-time towards the 5′ as well as towards the 3′ of the target sequence, also by trying to either expand the length of the ASO or by trying to shorten the sequence. Next, we will optimize each ASO regarding its backbone chemistry, testing for example phosphorodiamidate morpholine oligomers (PMO), peptide nucleic acids (PNA), and locked nucleic acids (LNAs) (as outlined in Dhuri 2020 et al.).

To analyse the exon skipping efficiency, we will transfect human cell lines (HEK293 and SH-SY5Y) and harvest RNA 48 hours after transfection using standard RNA extraction protocols. After preparation of cDNA, we will perform RT-PCR analysis using primers specific for exons flanking Ex23, and then quantify the level of transcript for which successful exclusion of Ex23 has occurred.

### Results:

We will identify the one or more ASOs optimized to apply to a human cell for skipping SORL1 Ex 23.

### Conclusion:

The identified one or more ASOs will be used in studies to test clinical efficacy.

### Example 10: Preparation of SORL1-Associated Alzheimer's Disease (SAAD) Cell Model (iPSC)

### Aim:

We aim to generate induced pluripotent stem cell (iPSC) line with genetic engineered variants of SORL1 Ex23.

### Material and Methods:

Amyloid beta (Aβ) peptides, pathological hallmarks of Alzheimers disease, will be determined using mesoscale discovery assays and endosome size using immunocytochemistry applying a Rab5 antibody and quantification of Rab5-positive structures from confocal images using ImageJ plugin.

### Results:

Based on our studies in N2a cells with SORL1 variants D1105H and R1080C (e.g. in Example 3), where we provide evidence that these are pathogenic variants, we will introduce these two mutations individually following standard protocols for guide-RNA and CRIPS-Cas9 methodologies. The introduction of mutations will be validated using sequencing, and clones expressing mutant protein selected.

Each cell line will be used to generated human neurons following published differentiation protocols, and subsequent be ‘phenotyped’ by assays for measurements of Amyloid beta secretion and endosome swelling, following standard protocols.

### Conclusion:

We will generate two iPSC models each carrying a pathogenic SORL1 variants that reside in Ex23, and establish disease phenotypes based on Rab5-positive endosome structures and endosomal processing of Amyloid precursor protein into the Amyloid beta peptide. These cell models will be used for efficacy experiments.

### Example 11: Preparation of Lymphoblasts Disease Model

### Aim:

We aim to generate lymphoblast cells derived from AD patients and carriers of SORL1 Ex23 pathogenic variants

### Material and Methods:

The SORLA maturation will be determined using WB analysis of cell lysates comparing cells from carriers of pathogenic variants to control cells.

### Results:

Pathogenic variants of SORL1 lead to maturation defective proteins. Western blot analysis of lysates of lymphoblasts from control individuals with wild-type SORL1 will show both mature and immature SORLA protein, whereas the lysates of cells isolated from carriers of a pathogenic SORL1 variant will have relatively more immature and less mature SORLA protein.

Moreover, as shedding that produces the shed sSORLA fragment only occurs for mature SORLA, the level of sSORLA in media from these cells will show low levels of sSORLA from lymphoblasts from SORL1 variant carriers in comparison to cells from wild-type SORL1 persons.

### Conclusion:

We will generate patient-derived lymphoblast cell lines from individuals that carry a pathogenic SORL1 variant in Ex23, and establish a disease phenotype based on SORLA maturation and secretion.

### Example 12: Establish Efficacy Levels Needed to Rescue AD Phenotypes of iPSC Disease Models

### Aim:

The aim is to determine the level of Ex23 skipping needed to revert a cellular disease phenotype to become indistinguishable for control cells with wild-type SORL1.

### Material and Methods:

We will prepare neurons from iPSC cells with SORL1 Ex23 pathogenic mutation and isogenic control cells, and then treat cells with our lead ASO(s) that induce skipping of the SORL1 Ex23. We will perform a titration of our ASO(s) using concentrations in the range from 2 nM to 500 nM, as well as treatments for a number of variable time courses.

### Results:

In cells with mutant SORL1, we will see a gradual rescue of the disease phenotype, with the largest effect for cells with the highest degree of obtained exon skipping. The level of Amyloid beta secretion and the size of Rab5-positive endosomes will be used as parameters of disease phenotypes. The degree of exon skipping will be evaluated using a qPCR assay that detect the Ex23 deleted transcript.

### Conclusion:

From these experiments we will be able to correlate the effect on disease phenotype and the level of induced SORL1 exon skipping. We will identify the efficacy we need of an ASO that induces skipping of a mutated SORL1 Ex23 to revert the cellular phenotype of an iPSC model to become similar to non-disease cells.

### Example 13: Establish Efficacy Levels Needed to Rescue AD Phenotypes of Lymphoblast Disease Models

### Aim:

The aim is to determine the level of Ex23 skipping needed to revert a cellular disease phenotype to become indistinguishable for control cells with wild-type SORL1.

### Material and Methods:

We will treat lymphoblast cells (from carriers and non-carriers) with our lead ASO(s) that induce skipping of the SORL1 Ex23. We will perform a titration of our ASO(s) using concentration in the range from 2 nM to 500 nM, as well as treatments for a number of variable time courses. In cells with mutant SORL1, we will see a gradual rescue of the disease phenotype, with the largest effect for cells with the highest degree of obtained exon skipping. The level of SORLA maturation and sSORLA secretion will be used as parameters of disease phenotypes. The degree of exon skipping will be evaluated using a qPCR assay that detect the Ex23 deleted transcript.

### Results:

From these experiments we will be able to correlate the effect on disease phenotype and the level of induced SORL1 exon skipping.

### Conclusion:

We will identify the efficacy we need of an ASO that induces skipping of a mutated SORL1 Ex23 to revert the cellular phenotype of a lymphoblast model to become similar to non-diseased cells.

### Sequences

TABLE 6
Sequences

| SEQ ID | | |
|---|---|---|
| NO | Name | Sequence |
| 1 | Exon 23 | agaacacctgtcttcgcaaccagtatcgctgcagcaacgggaa |
| | SORL1 | ctgtatcaacagcatttggtggtgtgactttgacaacgactgt |
| | | ggagacatgagcgatgagagaaactgcc |
| 2 | Exon 24 | ctaccaccatctgtgacctggacacccagtttcgttgccagga |
| | SORL1 | gtctgggacttgtatcccactgtcctataaatgtgaccttgag |
| | | gatgactgtggagacaacagtgatgaaagtcattgtg |
| 3 | Exon 25 | aaatgcaccagtgccggagtgacgagtacaactgcagttccgg |
| | SORL1 | catgtgcatccgctcctcctgggtatgtgacggggacaacgac |
| | | tgcagggactggtctgatgaagccaactgtaccg |
| 4 | Exon 26 | ccatctatcacacctgtgaggcctccaacttccagtgccgaaa |
| | SORL1 | cgggcactgcatcccccagcggtgggcgtgtgacggggatacg |
| | | gactgccaggatggttccgatgaggatccagtcaactgtg |
| 5 | Exon 27 | agaagaagtgcaatggattccgctgcccaaacggcacttgcat |
| | SORL1 | cccatccagcaaacattgtgatggtctgcgtgattgctctgat |
| | | ggctccgatgaacagcactgcg |
| 6 | Exon 28 | agcccctctgtacgcacttcatggactttgtgtgtaagaaccg |
| | SORL1 | ccagcagtgcctgttccactccatggtctgtgacggaatcatc |
| | | cagtgccgcgacgggtccgatgaggatgcggcgtttgcaggat |
| | | gct |
| 7 | Exon 29 | cccaagatcctgagttccacaaggtatgtgatgagttcggttt |
| | SORL1 | ccagtgtcagaatggagtgtgcatcagtttgatttggaagtgc |
| | | gacgggatggatgattgcggcgattattctgatgaagccaact |
| | | gcg |
| 8 | Exon 30 | aaaaccccacagaagccccaaactgctcccgctacttccagtt |
| | SORL1 | tcggtgtgagaatggccactgcatccccaacagatggaaatgt |
| | | gacagggagaacgactgtggggactggtctgatgagaaggatt |
| | | gtggag |
| | Exon 31 | attcacatattcttcccttctcgactcctgggccctccacgtg |
| | SORL1 | tctgcccaattactaccgctgcagcagtgggacctgcgtgatg |
| | | gacacctgggtgtgcgacgggtaccgagattgtgcagatggct |
| | | ctgacgaggaagcctgccccttgcttg |
| 10 | Exon 32 | caaacgtcactgctgcctccactcccacccaacttgggcgatg |
| | SORL1 | tgaccgatttgagttcgaatgccaccaaccgaagacgtgtatt |
| | | cccaactggaagcgctgtgacggccaccaagattgccaggatg |
| | | gccgggacgaggccaattgcc |
| 11 | Exon 33 | ccacacacagcaccttgacttgcatgagcagggagttccagtg |
| | SORL1 | cgaggacggggaggcctgcattgtgctctcggagcgctgcgac |
| | | ggcttcctggactgctcggacgagagcgatgaaaaggcctgca |
| | | gtg |
| 12 | RNA target | gauggguagAGAACACCUGUC |
| | sequence | |
| | ASO23.1 | |
| 13 | RNA target | AACGGGAACUGUAUCAACAGC |
| | sequence | |
| | ASO23.2 | |
| 14 | RNA target | UAUCGCUGCAGCAACGGGAAC |
| | sequence | |
| | ASO23.3 | |
| 15 | RNA target | GGUGUGACUUUGACAACGACU |
| | sequence | |
| | ASO23.4 | |
| 16 | ASO | GACAGGUGUUCUCUACCCAUC |
| | sequence | |
| | ASO23.1 | |
| 17 | ASO | GCUGUUGAUACAGUUCCCGUU |
| | sequence | |
| | ASO23.2 | |
| 18 | ASO | GUUCCCGUUGCUGCAGCGAUA |
| | sequence | |
| | ASO23.3 | |
| 19 | ASO | AGUCGUUGUCAAAGUCACACC |
| | sequence | |
| | ASO23.4 | |
| 20 | RNA target | ucuauuucagAGAAGAAGUGC |
| | sequence | |
| | ASO27.1 | |
| 21 | RNA target | GGAUUCCGCUGCCCAAACGGC |
| | sequence | |
| | ASO27.2 | |
| 22 | RNA target | guguuguugaauucuauuuca |
| | sequence | |
| | ASO27.3 | |
| 23 | RNA target | CUUGCAUCCCAUCCAGCAAAC |
| | sequence | |
| | ASO27.4 | |
| 24 | ASO | GCACUUCUUCUCUGAAAUAGA |
| | sequence | |
| | ASO27.1 | |
| 25 | ASO | GCCGUUUGGGCAGCGGAAUCC |
| | sequence | |
| | ASO27.2 | |
| 26 | ASO | UGAAAUAGAAUUCAACAACAC |
| | sequence | |
| | ASO27.3 | |
| 27 | ASO | GUUUGCUGGAUGGGAUGCAAG |
| | sequence | |
| | ASO27.4 | |
| 28 | RNA target | agaagccucucuguguuucag |
| | sequence | |
| | ASO33.1 | |
| 29 | RNA target | uguuucagCCACACACAGCAC |
| | sequence | |
| | ASO33.2 | |
| 30 | RNA target | GCACCUUGACUUGCAUGAGCA |
| | sequence | |
| | ASO33.3 | |
| 31 | RNA target | CCAGUGCGAGGACGGGGAGGC |
| | sequence | |
| | ASO33.4 | |
| 32 | ASO | CUGAAACACAGAGAGGCUUCU |
| | sequence | |
| | ASO33.1 | |
| 33 | ASO | GUGCUGUGUGUGGCUGAAACA |
| | sequence | |
| | ASO33.2 | |
| 34 | ASO | UGCUCAUGCAAGUCAAGGUGC |
| | sequence | |
| | ASO33.3 | |
| 35 | ASO | GCCUCCCCGUCCUCGCACUGG |
| | sequence | |
| | ASO33.4 | |
| 36 | Exon 23 | cgauaguuac uaaagggagg gaaacucaaa agagaaagac |
| | transcript | cuguggucca gcaguaagaa uaauauuggu uucauuuccu |
| | with 100+ | ccccugccgc acucugaugg guagagaaca ccugucuucg |
| | bp | caaccaguau cgcugcagca acgggaacug uaucaacagc |
| | flanking | auuugguggu gugacuuuga caacgacugu ggagacauga |
| | intron | gcgaugagag aaacugccgu gagucuucug gauuggacgu |
| | | uaagcacuua ccauuacuca gaagccuggu uggcucuucc |
| | | caggcugagg gccuaagguc uagggcgagg gccacccaug |
| 37 | Exon 24 | uuaauaccug cuuucuucua uuuuuuaauu gguuuuccag |
| | transcript | uaggauguuu acauuugugg gaaaucaauu guaccuaagg |
| | with 100+ | aauauguugu cuuucuaucc cauuuuagcu accaccaucu |
| | bp | gugaccugga cacccaguuu cguugccagg agucugggac |
| | flanking | uuguauccca cuguccuaua aaugugaccu ugaggaugac |
| | intron | uguggagaca acagugauga aagucauugu gguaagggga |
| | | caucacaucc ugaaacccug cucuggagag gggggucauu |
| | | gugcucccuc acagggcugu acugguaugu acugguuuuc |
| | | ugauagccgu cucaggauuu |
| 38 | Exon 25 | auuggugauc acggguccau cuccauccuu uaugagagcu |
| | transcript | uugacaccag agacaaaauu cugaacaagc uuuuguccuc |
| | with 100+ | accucucugu uuauggucuc accugcagaa augcaccagu |
| | bp | gccggaguga cgaguacaac ugcaguuccg gcaugugcau |
| | flanking | ccgcuccucc uggguaugug acggggacaa cgacugcagg |
| | intron | gacuggucug augaagccaa cuguaccggu caguacuucc |
| | | uggacucagu ugacagcacu cauccguuca ugcagugguu |
| | | aacauuauag cuuuaaacga ucggaaaauc uaggcucuga |
| | | gaauuagcuu |
| 39 | Exon 26 | ccuuauuccu accucaugca aaccucuuuu cagcccaccc |
| | transcript | ccuuggacag uugguggggg auggagauga ggggugugcg |
| | with 100+ | acugugucuc ucuucucugu uacagccauc uaucacaccu |
| | bp | gugaggccuc caacuuccag ugccgaaacg ggcacugcau |
| | flanking | cccccagcgg ugggcgugug acggggauac ggacugccag |
| | intron | gaugguuccg augaggaucc agucaacugu gguaaaugca |
| | | aauuccccag cucccucccu gagccucccc agugucugcu |
| | | guucaggaag agaggccaca gagagccagg ggaugaaaug |
| | | cuguucuccu augccuguau |
| 40 | Exon 27 | aauugcccuc cccaguggug guaccagugu guacucccgc |
| | transcript | cagcacugug ugugagugcc ugccuguuuc cuccucguca |
| | with 100+ | uucuucugug uuguugaauu cuauuucaga gaagaagugc |
| | bp | aauggauucc gcugcccaaa cggcacuugc aucccaucca |
| | flanking | gcaaacauug ugauggucug cgugauugcu cugauggcuc |
| | intron | cgaugaacag cacugcggug aguucauucc uugcccccag |
| | | gaagcacuca ggccuaguga uuaugaguga agagcguaua |
| | | uuggacuaaa uccuuucauu uccucaguac cugcuugugg |
| 41 | Exon 28 | uguugcuucc ugaagccaca ucuguacucg ugugcacuug |
| | transcript | cccagggcua gagggcccag ccagccgcag ugcucauggc |
| | with 100+ | cucuucccuu cucuggaucc cuuacagagc cccucuguac |
| | bp | gcacuucaug gacuuugugu guaagaaccg ccagcagugc |
| | flanking | cuguuccacu ccauggucug ugacggaauc auccagugcc |
| | intron | gcgacggguc cgaugaggau gcggcguuug caggaugcug |
| | | ugaguugggg caggcagggg aggugacuca cggucacuaa |
| | | agaacuugca uggggguuug gccacccugg gguugugucu |
| | | cuauuuaaua acugacaggu cuugguugag |
| 42 | Exon 29 | uuugccagaa cucacugcug uucccccuug cuucugggca |
| | transcript | ccacugcugc uucgaccccu cagcauggaa guuccuggac |
| | with 100+ | uucauggaug uuuguucccu cuguagccca agauccugag |
| | bp | uuccacaagg uaugugauga guucgguuuc cagugucaga |
| | flanking | auggagugug caucaguuug auuuggaagu gcgacgggau |
| | intron | ggaugauugc ggcgauuauu cugaugaagc caacugcggu |
| | | aagaugucca gucugccucc ucacauccua auccucuagu |
| | | uaacagugau gccugcaguu acagggacac ucaccggcaa |
| | | ccccacugca gacuuggccu |
| 43 | Exon 30 | guguggguug cccuggaacu ugggucugga ggaggaugcc |
| | transcript | uagaguugga gcccuguguu cacacugaug cagggaaagc |
| | with 100+ | aacugaugau gugguugauc ucugcagaaa accccacaga |
| | bp | agccccaaac ugcucccgcu acuuccaguu ucggugugag |
| | flanking | aauggccacu gcauccccaa cagauggaaa ugugacaggg |
| | intron | agaacgacug uggggacugg ucugaugaga aggauugugg |
| | | agguaagagg ccccuggggc cuggguuagc cccauaacca |
| | | agacacacca gaauaguucc accagcuaug caggaugugc |
| | | cuggcugauu ccguguuuug gggggcauau |
| 44 | Exon 31 | agcagguaca gcuaaaacug ggacauccgc acuagguuug |
| | transcript | ggacauauuu ggucuaagau caagcugcuu cuaauguuuu |
| | with 100+ | ggguuuccgu gcuugguguu uuucucagau ucacauauuc |
| | bp | uucccuucuc gacuccuggg cccuccacgu gucugcccaa |
| | flanking | uuacuaccgc ugcagcagug ggaccugcgu gauggacacc |
| | intron | ugggugugcg acggquaccg agauugugca gauggcucug |
| | | acgaggaagc cugccccuug cuuggugagu ucuggcccag |
| | | guccucuccu gugguaccug cuauugugga gaggaccuuc |
| | | ugggggugug cucugggcac aauccaaccg ggccccaugc |
| | | ccugcugucu |
| 45 | Exon 32 | ucccauugua auuucuaaag caccguaauc uccuagcaua |
| | transcript | uugauugguu gcuguuauug gccagcuccc ucaauauuaa |
| | with 100+ | aaaguaaauu uuaaaaaucu uuuauuuuag caaacgucac |
| | bp | ugcugccucc acucccaccc aacuugggcg augugaccga |
| | flanking | uuugaguucg aaugccacca accgaagacg uguauuccca |
| | intron | acuggaagcg cugugacggc caccaagauu gccaggaugg |
| | | ccgggacgag gccaauugcc gugaguaguc agagagcccu |
| | | ucacccccug ggcacguuuc ugcagagagc acaguucugg |
| | | ugcuucugcu ucauuucugg aucagcacac gccugugugu |
| | | gagucugugu |
| 46 | Exon 33 | uugaagcaga agccaauuag uacuuugccu aaaugggaau |
| | transcript | aaacuugggc ccagccuuua guaccauacg gccccucccc |
| | with 100+ | guggacuuaa gaagccucuc uguguuucag ccacacacag |
| | bp | caccuugacu ugcaugagca gggaguucca gugcgaggac |
| | flanking | ggggaggccu gcauugugcu cucggagcgc ugcgacggcu |
| | intron | uccuggacug cucggacgag agcgaugaaa aggccugcag |
| | | uggugagugc cgguccacgg gcugggcugg gcugggcugg |
| | | gcugggaggc ucgcuuaccc cagggccccu ccuguguaga |
| | | ccuugagcua ggacccuuuu gaacuguugc |
| 47 | Forward - | ACACTGGAAGCAATGCCTGT |
| | Primer | |
| | Ex23 | |
| 48 | Reverse - | CGGCACTGGTGCATTTCAC |
| | Primer | |
| | Ex23 | |
| 49 | Forward - | AGTAGCGTTCGCCCGAACATGGCGACACGGAGCAGC |
| | SORL1 E1 | |
| | ΔE23-Δ28, | |
| | and | |
| | ΔE30-ΔE33 | |
| 50 | Reverse - | CTAGTGAGGAAAGCTCTTTCAGGCTATCACCATGGG |
| | SORL1 | |
| | E48 - for | |
| | AE23-A27 | |
| 51 | Reverse - | TTCTAGTGAGGAAAGCTCTTTCAGGCTATCACCATGGG |
| | SORL1 | |
| | E48 - for | |
| | ΔE29Δ30 | |
| 52 | Reverse - | TAGTGAGGAAAGCTCTTTCAGGCTATCACCATGGG |
| | SORL1 | |
| | E48 - for | |
| | ΔE28, and | |
| | ΔE30-Δ33 | |
| 53 | Reverse - | GGTCACAGATGGTGGTAGCTTCTTTGACACAGGTATTGTTCTT |
| | SORL1- | GAGC |
| | ΔE23 - | |
| | E1-22 | |
| 54 | Forward - | TACCTGTGTCAAAGAAGCTACCACCATCTGTGACC |
| | SORL1- | |
| | ΔE23 - | |
| | E24-48 | |
| 55 | Reverse - | TCCGGCACTGGTGCATTTGGCAGTTTCTCTCATCGCTCATG |
| | SORL1- | |
| | AE24 - | |
| | E1-23 | |
| 56 | Forward - | CGATGAGAGAAACTGCCAAATGCACCAGTGCCGGAG |
| | SORL1- | |
| | ΔE24 - | |
| | E25-48 | |
| 57 | Reverse - | CACAGGTGTGATAGATGGCACAATGACTTTCATCACTGTTGTC |
| | SORL1- | TCC |
| | ΔE25 - | |
| | E1-24 | |
| 58 | Forward - | TGATGAAAGTCATTGTGCCATCTATCACACCTGTG |
| | SORL1- | |
| | ΔE25 - | |
| | E26-48 | |
| 59 | Reverse - | ATCCATTGCACTTCTTCTCGGTACAGTTGGCTTCATCAGAC |
| | SORL1- | |
| | ΔE26 - | |
| | E1-25 | |
| 60 | Forward - | TGAAGCCAACTGTACCGAGAAGAAGTGCAATGGATTC |
| | SORL1- | |
| | ΔE26 - | |
| | E27-48 | |
| 61 | Reverse - | AGTGCGTACAGAGGGGCTCACAGTTGACTGGATCCTCATCG |
| | SORL1- | |
| | ΔE27 - | |
| | E1-26 | |
| 62 | Forward - | GGATCCAGTCAACTGTGAGCCCCTCTGTACGCACTTC |
| | SORL1- | |
| | ΔE27 - | |
| | E28-48 | |
| 63 | Reverse - | GGAACTCAGGATCTTGGGCGCAGTGCTGTTCATCGG |
| | SORL1- | |
| | ΔE28 - | |
| | E1-27 | |
| 64 | Forward - | CGATGAACAGCACTGCGCCCAAGATCCTGAGTTCC |
| | SORL1- | |
| | ΔE28 - | |
| | E29-48 | |
| 65 | Reverse - | GAAGGGAAGAATATGTGAATAGCATCCTGCAAACGCC |
| | SORL1- | G |
| | ΔE29 - | |
| | E1-28 | |
| 66 | Forward - | TGCGGCGTTTGCAGGATGCTATTCACATATTCTTCCCT |
| | SORL1- | TCTC |
| | ΔE29 - | |
| | E30-48 | |
| 67 | Reverse - | AGGGAAGAATATGTGAATCGCAGTTGGCTTCATCAGAA |
| | SORL1- | TAATCG |
| | ΔE30 - | |
| | E1-29 | |
| 68 | Forward - | TGATGAAGCCAACTGCGATTCACATATTCTTCCCTTCTC |
| | SORL1- | |
| | ΔE30 - | |
| | E31-48 | |
| 69 | Reverse - | AGGCAGCAGTGACGTTTGCTCCACAATCCTTCTCATCA |
| | SORL1- | GACC |
| | ΔE31 - | |
| | E1-30 | |
| 70 | Forward - | TGAGAAGGATTGTGGAGCAAACGTCACTGCTGCCTC |
| | SORL1- | |
| | ΔE31 - | |
| | E32-48 | |
| 71 | Reverse - | TCAAGGTGCTGTGTGTGGCAAGCAAGGGGCAGGCTTC |
| | SORL1- | |
| | AE32 - | |
| | E1-31 | |
| 72 | Forward - | AGCCTGCCCCTTGCTTGCCACACACAGCACCTTGAC |
| | SORL1- | |
| | AE32 - | |
| | E33-48 | |
| 73 | Reverse - | TGTACACAGTCAACTCATGGCAATTGGCCTCGTCCC |
| | SORL1- | |
| | AE33 - | |
| | E1-32 | |
| 74 | Forward - | GGACGAGGCCAATTGCCATGAGTTGACTGTGTACAAA |
| | SORL1- | G |
| | AE33 - | |
| | E34-48 | |
| 75 | Forward - | GCAGTAGCGTTCGCCCGAACATGGCGACACGGAGCAG |
| | SORL1- | C |
| | ΔE29Ε30 | |
| | E1-28 | |
| 76 | E23-3′ | tcccctgccgcactctgatgggtagagaacacctgtcttcgca |
| | Splice | accagta |
| | acceptor | |
| 77 | E23-5′ | gacatgagcgatgagagaaactgccgtgagtcttctggattgg |
| | Splice | acgttaa |
| | acceptor | |
| 78 | E24-3′ | atgttgtctttctatcccattttagctaccaccatctgtgacc |
| | Splice | tggacac |
| | acceptor | |
| 79 | E24-5′ | gacaacagtgatgaaagtcattgtggtaaggggacatcacatc |
| | Splice | ctgaaac |
| | acceptor | |
| 80 | E25-3′ | tctctgtttatggtctcacctgcagaaatgcaccagtgccgga |
| | Splice | gtgacga |
| | acceptor | |
| 81 | E25-5′ | tggtctgatgaagccaactgtaccggtcagtacttcctggact |
| | Splice | cagttga |
| | acceptor | |
| 82 | E26-3′ | actgtgtctctcttctctgttacagccatctatcacacctgtg |
| | Splice | aggcctc |
| | acceptor | |
| 83 | E26-5′ | tccgatgaggatccagtcaactgtggtaaatgcaaattcccca |
| | Splice | gctccct |
| | acceptor | |
| 84 | E27-3′ | tctgtgttgttgaattctatttcagagaagaagtgcaatggat |
| | Splice | tccgctg |
| | acceptor | |
| 85 | E27-5′ | gatggctccgatgaacagcactgcggtgagttcattccttgcc |
| | Splice | cccagga |
| | acceptor | |
| 86 | E28-3′ | cttcccttctctggatcccttacagagcccctctgtacgcact |
| | Splice | tcatgga |
| | acceptor | |
| 87 | E28-5′ | gaggatgcggcgtttgcaggatgctgtgagttggggcaggcag |
| | Splice | gggaggt |
| | acceptor | |
| 88 | E29-3′ | tcatggatgtttgttccctctgtagcccaagatcctgagttcc |
| | Splice | acaaggt |
| | acceptor | |
| 89 | E29-5′ | gattattctgatgaagccaactgcggtaagatgtccagtctgc |
| | Splice | ctcctca |
| | acceptor | |
| 90 | E30-3′ | ctgatgatgtggttgatctctgcagaaaaccccacagaagccc |
| | Splice | caaactg |
| | acceptor | |
| 91 | E30-5′ | tggtctgatgagaaggattgtggaggtaagaggcccctggggc |
| | Splice | ctgggtt |
| | acceptor | |
| 92 | E31-3′ | tttccgtgcttggtgtttttctcagattcacatattcttccct |
| | Splice | tctcgac |
| | acceptor | |
| 93 | E31-5′ | gacgaggaagcctgccccttgcttggtgagttctggcccaggt |
| | Splice | cctctcc |
| | acceptor | |
| 94 | E32-3′ | aaattttaaaaatcttttattttagcaaacgtcactgctgcct |
| | Splice | ccactcc |
| | acceptor | |
| 95 | E32-5′ | gatggccgggacgaggccaattgccgtgagtagtcagagagcc |
| | Splice | cttcacc |
| | acceptor | |
| 96 | E33-3′ | cttaagaagcctctctgtgtttcagccacacacagcaccttga |
| | Splice | cttgcat |
| | acceptor | |
| 97 | E33-5′ | gagagcgatgaaaaggcctgcagtggtgagtgccggtccacgg |
| | Splice | gctgggc |
| | acceptor | |

SEQ ID NO: 36 to SEQ ID NO: 46: Underlined nucleotides represent exons, non-underlined nucleotides represent part of the neighboring introns. Bold nucleotides represent the nucleotides at the boundary between an intron and an exon, i.e. the ends of an intron (ag/gt).

SEQ ID NO: 49 to SEQ ID NO: 75: PCR primers used for amplification of SORL1 fragments (from Example 2, Table 5). The following are explanatory examples of the naming used in the table above:
“Forward—SORL1 E1 ΔE23-Δ28, and ΔE30-ΔE33” means that the sequence is the Forward primer (5′-3′) for SORL1-ΔE23 (E1-22 PCR product), SORL1-ΔE24 (E1-23), SORL1-ΔE25 (E1-24), SORL1-ΔE26 (E1-25), SORL1-ΔE27 (E1-26), SORL1-ΔE28 (E1-27), SORL1-ΔE30 (E1-29), SORL1-ΔE31 (E1-30), SORL1-ΔE32 (E1-31), SORL1-ΔE33 (E1-32).
“Reverse—SORL1-ΔE23-E1-22” means that the sequence is the Reverse primer (5′-3′) for SORL1-ΔE23 (E1-22 PCR product).

### REFERENCES

Dhuri 2020, Antisense Oligonucleotides: An Emerging Area in Drug Discovery and Development, J Clin Med. 2020 Jun. 26; 9(6):2004. doi: 10.3390/jcm9062004.
Holstege 2020, Exome sequencing identifies novel AD-associated genes. 24 2020 Jul. 24, doi.org/10.1101/2020.07.22.2015925
Jacobsen 2001, Activation and functional characterization of the mosaic receptor SorLA/LR11, J Biol Chem. 2001 Jun. 22; 276(25):22788-96. doi: 10.1074/jbc.M100857200. Epub 2001 Apr. 9.
Piva 2009, SpliceAid: a database of experimental RNA target motifs bound by splicing proteins in humans, Bioinformatics. 2009 May 1; 25(9):1211-3. doi: 10.1093/bioinformatics/btp124. Epub 2009 Mar. 4.

1. An antisense oligonucleotide (ASO) that binds to and/or is complementary to a target site on the pre-mRNA of SORL1, wherein the nucleotide sequence of the target site is comprised in a nucleotide sequence selected from the group consisting of
SEQ ID NO: 36, SEQ ID NO: 37, SEQ ID NO: 38, SEQ ID NO: 39,
SEQ ID NO: 40, SEQ ID NO: 41, SEQ ID NO: 42, SEQ ID NO: 43,
SEQ ID NO: 44, SEQ ID NO: 45 and SEQ ID NO: 46,
or a nucleotide sequence having at least 80% sequence identity or homology to a nucleotide sequence selected from the group consisting of
SEQ ID NO: 36, SEQ ID NO: 37, SEQ ID NO: 38, SEQ ID NO: 39,
SEQ ID NO: 40, SEQ ID NO: 41, SEQ ID NO: 42, SEQ ID NO: 43,
SEQ ID NO: 44, SEQ ID NO: 45 and SEQ ID NO: 46,
for example at least 80%, 85%, 90%, 95%, 98% or 99% sequence identity or homology thereto.

2. The oligonucleotide according to claim 1, wherein the oligonucleotide binding to and/or complementary to a target site causes exon skipping of an exon encoding a complement-type repeat (CR) domain of SORLA, wherein the exon comprises or consists of a nucleotide sequence selected from the group consisting of
exon 23 having a sequence of SEQ ID NO: 1,
exon 24 having a sequence of SEQ ID NO: 2,
exon 25 having a sequence of SEQ ID NO: 3,
exon 26 having a sequence of SEQ ID NO: 4,
exon 27 having a sequence of SEQ ID NO: 5,
exon 28 having a sequence of SEQ ID NO: 6,
exon 29 having a sequence of SEQ ID NO: 7,
exon 30 having a sequence of SEQ ID NO: 8,
exon 31 having a sequence of SEQ ID NO: 9,
exon 32 having a sequence of SEQ ID NO: 10, and
exon 33 having a sequence of SEQ ID NO: 11,
or a nucleotide sequence having at least 80% sequence identity or homology to any one of SEQ ID NO: 1 to SEQ ID NO: 11, for example at least 80%, 85%, 90%, 95%, 98% or 99% sequence identity or homology thereto.

3. The oligonucleotide according to any one of the preceding claims, wherein the oligonucleotide is between 10 to 30 nucleotides in length, such as 10 to 13 nucleotides, such as 10 to 16 nucleotides, such as 10 to 19 nucleotides, such as 10 to 22 nucleotides, such as 10 to 23 nucleotides, such as 10 to 26 nucleotides, such as 10 to 29 nucleotides, such as 10 to 30 nucleotides.

4. The oligonucleotide according to any one of the preceding claims, wherein the oligonucleotide is at least 10 nucleotides long, such as at least 12 nucleotides, and/or at least 14 nucleotides, and/or at least 16 nucleotides and/or at least 18 nucleotides, and/or at least 20, and/or at least 22 nucleotides, and/or at least 24 nucleotides, and/or at least 26 nucleotides, and/or at least 28 nucleotides, and/or at least 30 nucleotides long.

5. The oligonucleotide according to any one of the preceding claims, wherein the oligonucleotide is 21 nucleotides long.

6. The oligonucleotide according to any one of the preceding claims, wherein the oligonucleotide has a GC-content of 40 to 60%, such as 45 to 55%.

7. The oligonucleotide according to any one of the preceding claims, wherein the oligonucleotide comprises a backbone comprising of phosphorothioate (PS).

8. The oligonucleotide according to any one of the preceding claims, wherein the oligonucleotide further comprises modifications at at least one nucleotide position, or at each nucleotide position.

9. The oligonucleotide according to any one of the preceding claims, wherein the modification is a modification of the nucleic acid backbone, the nucleobase, the ribose sugar and/or 2′-ribose substitutions.

10. The oligonucleotide according to any one of the preceding claims, wherein the oligonucleotide comprises a 2′-O-methoxyethyl sugar modification.

11. The oligonucleotide according to any one of the preceding claims, wherein the oligonucleotide comprises a 2′-O-methy ribose modification.

12. The oligonucleotide according to any one of the preceding claims, wherein the target site is at the 3′ splice site boundary, at the 5′ splice site boundary and/or at an exonic splice enhancer (ESE) site.

13. The oligonucleotide according to any one of the preceding claims, wherein the target site consists or comprises of a nucleotide sequence selected from the group consisting of
SEQ ID NO: 12, SEQ ID NO: 13, SEQ ID NO: 14, SEQ ID NO: 15,
SEQ ID NO: 20, SEQ ID NO: 21, SEQ ID NO: 22, SEQ ID NO: 23,
SEQ ID NO: 28, SEQ ID NO: 29, SEQ ID NO: 30, SEQ ID NO: 31,
SEQ ID NO: 36, SEQ ID NO: 37, SEQ ID NO: 38, SEQ ID NO: 39,
SEQ ID NO: 40, SEQ ID NO: 41, SEQ ID NO: 42, SEQ ID NO: 43,
SEQ ID NO: 44, SEQ ID NO: 45 and SEQ ID NO: 46,
or a nucleotide sequence having at least 80% sequence identity or homology to a nucleotide sequence selected from the group consisting of
SEQ ID NO: 12, SEQ ID NO: 13, SEQ ID NO: 14, SEQ ID NO: 15,
SEQ ID NO: 20, SEQ ID NO: 21, SEQ ID NO: 22, SEQ ID NO: 23,
SEQ ID NO: 28, SEQ ID NO: 29, SEQ ID NO: 30, SEQ ID NO: 31,
SEQ ID NO: 36, SEQ ID NO: 37, SEQ ID NO: 38, SEQ ID NO: 39,
SEQ ID NO: 40, SEQ ID NO: 41, SEQ ID NO: 42, SEQ ID NO: 43,
SEQ ID NO: 44, SEQ ID NO: 45 and SEQ ID NO: 46,
for example at least 80%, 85%, 90%, 95%, 98% or 99% sequence identity or homology thereto.

14. The oligonucleotide according to any one of the preceding claims, wherein the oligonucleotide consists or comprises of a nucleotide sequence selected from the group consisting of
SEQ ID NO: 16, SEQ ID NO: 17, SEQ ID NO: 18, SEQ ID NO: 19,
SEQ ID NO: 24, SEQ ID NO: 25, SEQ ID NO: 26, SEQ ID NO: 27,
SEQ ID NO: 32, SEQ ID NO: 33, SEQ ID NO: 34 and SEQ ID NO: 35
or a nucleotide sequence having at least 80% sequence identity or homology to a nucleotide sequence selected from the group consisting of
SEQ ID NO: 16, SEQ ID NO: 17, SEQ ID NO: 18, SEQ ID NO: 19,
SEQ ID NO: 24, SEQ ID NO: 25, SEQ ID NO: 26, SEQ ID NO: 27,
SEQ ID NO: 32, SEQ ID NO: 33, SEQ ID NO: 34 and SEQ ID NO: 35,
for example at least 80%, 85%, 90%, 95%, 98% or 99% sequence identity or homology thereto.

15. The oligonucleotide according to any one of the preceding claims, wherein the oligonucleotide hybridizes specifically under high stringency solution hybridization conditions to target site.

16. The oligonucleotide according to any one of the preceding claims, wherein upon binding to the target site, the oligonucleotide prevents splicing factors from binding.

17. The oligonucleotide according to any one of the preceding claims, wherein the oligonucleotide is conjugated to a moiety or to a nanoparticle formulation.

18. The oligonucleotide according to any one of the preceding claims, wherein the moiety is a cell-targeting moiety and/or a cell-penetrating moiety.

19. The oligonucleotide according to any one of the preceding claims, wherein the oligonucleotide is conjugated to Triantennary N-acetylgalactosamine (GalNAc) moiety and/or a peptide.

20. The oligonucleotide according to any one of the preceding claims, wherein the oligonucleotide is targeted to a 5′ splice site, a 3′ splice site and/or an exonic splice enhancer site (ESE).

21. The oligonucleotide according to any one of the preceding claims, wherein the oligonucleotide further comprises at least one additional nucleotide, at least two additional nucleotides, at least three additional nucleotides, at one or both ends of the oligonucleotide.

22. A composition comprising the oligonucleotide according to any one of the preceding claims.

23. The composition according to claim 22, wherein the composition is a pharmaceutical composition.

24. The composition according to any one of claims 22 to 23, wherein the composition further comprises a pharmaceutically acceptable carrier.

25. The composition according to claim 22 comprising one or more of said oligonucleotides.

26. An antisense oligonucleotide (ASO) according to any one of claims 1 to 21 and/or the composition according to any one of claims 22 to 25, for use in medicine.

27. An antisense oligonucleotide (ASO) according to any one of claims 1 to 21 and/or the composition according to any one of claims 22 to 25, for use in the prevention, treatment and/or alleviation of Alzheimer's Disease (AD), or a disease or disorder associated with Alzheimer's Disease.

28. The antisense oligonucleotide according to any one of claims 26 to 27, wherein an effective amount of the oligonucleotide is administered to the eye, to the spinal cord, to cerebrospinal fluid, to the brain and/or to the liver.

29. The antisense oligonucleotide according to any one of claims 27 to 28, wherein an effective amount of the oligonucleotide is administered to an individual when a relative of said individual is diagnosed with Alzheimer's disease.

30. Use of an antisense oligonucleotide according to any one of claims 1 to 21 or of a composition according to any one of claims 22 to 25 in the manufacture of a medicament for the treatment of Alzheimer's Disease (AD), or a disease or disorder associated with Alzheimer's Disease.

31. A method for mediating exon skipping in SORL1 transcripts in a cell, tissue or organ using the antisense oligonucleotide (ASO) according to any one of claims 1 to 21 and/or the composition according to any one of claims 22 to 25,
where the exon is selected from the group consisting of exon 23, exon 24, exon 25, exon 26, exon 27, exon 28, exon 29, exon 30, exon 31, exon 32 and exon 33 of SORL1.

32. The method according to claim 31, wherein one ASO is used.

33. The method according to any one of claims 31 to 32, wherein more than one ASO is used, for example two ASOs, for example three ASOs, for example five ASOs, for example six ASOs.

34. The method according to any one of claims 31 to 33, wherein exon skipping of one exon is mediated.

35. The method according to any one of claims 31 to 34, wherein exon skipping of more than one exon is mediated, for example two exons, for example three exons, for example four exons.

36. A method of determining the efficiency of ASO mediated SORL1 exon skipping in a subject, the method comprising the following steps:
a) analyzing the levels of shed SORLA in a first sample comprising cerebrospinal fluid derived from a patient, obtained before treatment with an ASO,
b) analyzing the levels of shed SORLA in a second sample comprising cerebrospinal fluid derived from the same patient as in a), obtained after treatment with an ASO,
c) comparing the level of shed SORLA in the samples of a) and b),
thereby determining exon skipping if the level of shed SORLA in b) is higher than in a).

37. The method according to claim 36, optionally comprising the step of obtaining sample b) at several time points after treatment with an ASO, thus monitoring the efficiency of ASO mediated exon skipping over time.

38. A method for testing if a patient identified with a SORL1 mutation will benefit from treatment with an ASO mediating exon skipping, the method comprising the steps of:
a) Identifying a mutation in any one of exon 23 to 33 of SORL1,
b) Introducing the SORL1 mutation identified in the patient into a cell line,
c) Selecting one or more ASO that targets the exon carrying the identified mutation,
d) Contacting a first aliquot of cells of with medium comprising the selected one or more ASO, and contacting a second aliquot of cells of with medium not comprising any ASO,
e) Analyzing the levels of shed SORLA in the first and the second aliquot,
f) Comparing the level of shed SORLA in the first and the second aliquot,
thereby determining that the patient will benefit from treatment with the one or more ASO if the level of shed SORLA in the first aliquot is higher than in the second aliquot.

39. The method according to any one of claims 36 to 38, wherein the method is an in-vitro method.

40. A method of producing an ASO suitable for treatment of a patient with Alzheimer's disease (AD), wherein the patient carries a mutation in an exon encoding a complement-type repeat (CR) domain of SORLA, the method comprising the following steps:
a) identifying, for example in silico identifying, an ASO according to any one of claims 1 to 21,
b) determining if the target site of the ASO comprises the mutation, or if the target size of the ASO does not comprise the mutation, and
thereby determining that the ASO that binds to a target site not comprising the mutation is suitable for treatment of a patient with AD.

41. The method according to claim 40, wherein the mutation is a calcium-cage-mutation or an odd-numbered cysteines-mutation.

42. The method according any one of claims 40 to 41, wherein the mutation is a substitution of arginine to cysteine at position 1080 (R1080C) of human SORL1, or wherein the mutation is a substitution of aspartic acid to histidine at position 1105 (C1105H) of human SORL1.


SEQUENCE LISTING (97 sequences)

SEQ ID NO: 1 - DNA, 114 bp, synthetic construct
  source 1..114: mol_type = other DNA
agaacacctgtcttcgcaaccagtatcgctgcagcaacgggaactgtatcaacagcatttggtggtgtgactttgacaacgactgtggagacatgagcgatgagagaaactgcc

SEQ ID NO: 2 - DNA, 123 bp, synthetic construct
  source 1..123: mol_type = other DNA
ctaccaccatctgtgacctggacacccagtttcgttgccaggagtctgggacttgtatcccactgtcctataaatgtgaccttgaggatgactgtggagacaacagtgatgaaagtcattgtg

SEQ ID NO: 3 - DNA, 120 bp, synthetic construct
  source 1..120: mol_type = other DNA
aaatgcaccagtgccggagtgacgagtacaactgcagttccggcatgtgcatccgctcctcctgggtatgtgacggggacaacgactgcagggactggtctgatgaagccaactgtaccg

SEQ ID NO: 4 - DNA, 126 bp, synthetic construct
  source 1..126: mol_type = other DNA
ccatctatcacacctgtgaggcctccaacttccagtgccgaaacgggcactgcatcccccagcggtgggcgtgtgacggggatacggactgccaggatggttccgatgaggatccagtcaactgtg

SEQ ID NO: 5 - DNA, 108 bp, synthetic construct
  source 1..108: mol_type = other DNA
agaagaagtgcaatggattccgctgcccaaacggcacttgcatcccatccagcaaacattgtgatggtctgcgtgattgctctgatggctccgatgaacagcactgcg

SEQ ID NO: 6 - DNA, 132 bp, synthetic construct
  source 1..132: mol_type = other DNA
agcccctctgtacgcacttcatggactttgtgtgtaagaaccgccagcagtgcctgttccactccatggtctgtgacggaatcatccagtgccgcgacgggtccgatgaggatgcggcgtttgcaggatgct

SEQ ID NO: 7 - DNA, 132 bp, synthetic construct
  source 1..132: mol_type = other DNA
cccaagatcctgagttccacaaggtatgtgatgagttcggtttccagtgtcagaatggagtgtgcatcagtttgatttggaagtgcgacgggatggatgattgcggcgattattctgatgaagccaactgcg

SEQ ID NO: 8 - DNA, 135 bp, synthetic construct
  source 1..135: mol_type = other DNA
aaaaccccacagaagccccaaactgctcccgctacttccagtttcggtgtgagaatggccactgcatccccaacagatggaaatgtgacagggagaacgactgtggggactggtctgatgagaaggattgtggag

SEQ ID NO: 9 - DNA, 156 bp, synthetic construct
  source 1..156: mol_type = other DNA
attcacatattcttcccttctcgactcctgggccctccacgtgtctgcccaattactaccgctgcagcagtgggacctgcgtgatggacacctgggtgtgcgacgggtaccgagattgtgcagatggctctgacgaggaagcctgccccttgcttg

SEQ ID NO: 10 - DNA, 150 bp, synthetic construct
  source 1..150: mol_type = other DNA
caaacgtcactgctgcctccactcccacccaacttgggcgatgtgaccgatttgagttcgaatgccaccaaccgaagacgtgtattcccaactggaagcgctgtgacggccaccaagattgccaggatggccgggacgaggccaattgcc

SEQ ID NO: 11 - DNA, 132 bp, synthetic construct
  source 1..132: mol_type = other DNA
ccacacacagcaccttgacttgcatgagcagggagttccagtgcgaggacggggaggcctgcattgtgctctcggagcgctgcgacggcttcctggactgctcggacgagagcgatgaaaaggcctgcagtg

SEQ ID NO: 12 - RNA, 21 nt, synthetic construct
  source 1..21: mol_type = other RNA
gatgggtagagaacacctgtc

SEQ ID NO: 13 - RNA, 21 nt, synthetic construct
  source 1..21: mol_type = other RNA
aacgggaactgtatcaacagc

SEQ ID NO: 14 - RNA, 21 nt, synthetic construct
  source 1..21: mol_type = other RNA
tatcgctgcagcaacgggaac

SEQ ID NO: 15 - RNA, 21 nt, synthetic construct
  source 1..21: mol_type = other RNA
ggtgtgactttgacaacgact

SEQ ID NO: 16 - RNA, 21 nt, synthetic construct
  source 1..21: mol_type = other RNA
gacaggtgttctctacccatc

SEQ ID NO: 17 - RNA, 21 nt, synthetic construct
  source 1..21: mol_type = other RNA
gctgttgatacagttcccgtt

SEQ ID NO: 18 - RNA, 21 nt, synthetic construct
  source 1..21: mol_type = other RNA
gttcccgttgctgcagcgata

SEQ ID NO: 19 - RNA, 21 nt, synthetic construct
  source 1..21: mol_type = other RNA
agtcgttgtcaaagtcacacc

SEQ ID NO: 20 - RNA, 21 nt, synthetic construct
  source 1..21: mol_type = other RNA
tctatttcagagaagaagtgc

SEQ ID NO: 21 - RNA, 21 nt, synthetic construct
  source 1..21: mol_type = other RNA
ggattccgctgcccaaacggc

SEQ ID NO: 22 - RNA, 21 nt, synthetic construct
  source 1..21: mol_type = other RNA
gtgttgttgaattctatttca

SEQ ID NO: 23 - RNA, 21 nt, synthetic construct
  source 1..21: mol_type = other RNA
cttgcatcccatccagcaaac

SEQ ID NO: 24 - RNA, 21 nt, synthetic construct
  source 1..21: mol_type = other RNA
gcacttcttctctgaaataga

SEQ ID NO: 25 - RNA, 21 nt, synthetic construct
  source 1..21: mol_type = other RNA
gccgtttgggcagcggaatcc

SEQ ID NO: 26 - RNA, 21 nt, synthetic construct
  source 1..21: mol_type = other RNA
tgaaatagaattcaacaacac

SEQ ID NO: 27 - RNA, 21 nt, synthetic construct
  source 1..21: mol_type = other RNA
gtttgctggatgggatgcaag

SEQ ID NO: 28 - RNA, 21 nt, synthetic construct
  source 1..21: mol_type = other RNA
agaagcctctctgtgtttcag

SEQ ID NO: 29 - RNA, 21 nt, synthetic construct
  source 1..21: mol_type = other RNA
tgtttcagccacacacagcac

SEQ ID NO: 30 - RNA, 21 nt, synthetic construct
  source 1..21: mol_type = other RNA
gcaccttgacttgcatgagca

SEQ ID NO: 31 - RNA, 21 nt, synthetic construct
  source 1..21: mol_type = other RNA
ccagtgcgaggacggggaggc

SEQ ID NO: 32 - RNA, 21 nt, synthetic construct
  source 1..21: mol_type = other RNA
ctgaaacacagagaggcttct

SEQ ID NO: 33 - RNA, 21 nt, synthetic construct
  source 1..21: mol_type = other RNA
gtgctgtgtgtggctgaaaca

SEQ ID NO: 34 - RNA, 21 nt, synthetic construct
  source 1..21: mol_type = other RNA
tgctcatgcaagtcaaggtgc

SEQ ID NO: 35 - RNA, 21 nt, synthetic construct
  source 1..21: mol_type = other RNA
gcctccccgtcctcgcactgg

SEQ ID NO: 36 - RNA, 320 nt, synthetic construct
  source 1..320: mol_type = other RNA
cgatagttactaaagggagggaaactcaaaagagaaagacctgtggtccagcagtaagaataatattggtttcatttcctcccctgccgcactctgatgggtagagaacacctgtcttcgcaaccagtatcgctgcagcaacgggaactgtatcaacagcatttggtggtgtgactttgacaacgactgtggagacatgagcgatgagagaaactgccgtgagtcttctggattggacgttaagcacttaccattactcagaagcctggttggctcttcccaggctgagggcctaaggtctagggcgagggccacccatg

SEQ ID NO: 37 - RNA, 340 nt, synthetic construct
  source 1..340: mol_type = other RNA
ttaatacctgctttcttctattttttaattggttttccagtaggatgtttacatttgtgggaaatcaattgtacctaaggaatatgttgtctttctatcccattttagctaccaccatctgtgacctggacacccagtttcgttgccaggagtctgggacttgtatcccactgtcctataaatgtgaccttgaggatgactgtggagacaacagtgatgaaagtcattgtggtaaggggacatcacatcctgaaaccctgctctggagaggggggtcattgtgctccctcacagggctgtactggtatgtactggttttctgatagccgtctcaggattt

SEQ ID NO: 38 - RNA, 330 nt, synthetic construct
  source 1..330: mol_type = other RNA
attggtgatcacgggtccatctccatcctttatgagagctttgacaccagagacaaaattctgaacaagcttttgtcctcacctctctgtttatggtctcacctgcagaaatgcaccagtgccggagtgacgagtacaactgcagttccggcatgtgcatccgctcctcctgggtatgtgacggggacaacgactgcagggactggtctgatgaagccaactgtaccggtcagtacttcctggactcagttgacagcactcatccgttcatgcagtggttaacattatagctttaaacgatcggaaaatctaggctctgagaattagctt

SEQ ID NO: 39 - RNA, 340 nt, synthetic construct
  source 1..340: mol_type = other RNA
ccttattcctacctcatgcaaacctcttttcagcccacccccttggacagttggtgggggatggagatgaggggtgtgcgactgtgtctctcttctctgttacagccatctatcacacctgtgaggcctccaacttccagtgccgaaacgggcactgcatcccccagcggtgggcgtgtgacggggatacggactgccaggatggttccgatgaggatccagtcaactgtggtaaatgcaaattccccagctccctccctgagcctccccagtgtctgctgttcaggaagagaggccacagagagccaggggatgaaatgctgttctcctatgcctgtat

SEQ ID NO: 40 - RNA, 320 nt, synthetic construct [sequence omitted]

SEQ ID NO: 41 - RNA, 350 nt, synthetic construct
  source 1..350: mol_type = other RNA
tgttgcttcctgaagccacatctgtactcgtgtgcacttgcccagggctagagggcccagccagccgcagtgctcatggcctcttcccttctctggatcccttacagagcccctctgtacgcacttcatggactttgtgtgtaagaaccgccagcagtgcctgttccactccatggtctgtgacggaatcatccagtgccgcgacgggtccgatgaggatgcggcgtttgcaggatgctgtgagttggggcaggcaggggaggtgactcacggtcactaaagaacttgcatgggggtttggccaccctggggttgtgtctctatttaataactgacaggtcttggttgag

SEQ ID NO: 42 - RNA, 340 nt, synthetic construct
  source 1..340: mol_type = other RNA
tttgccagaactcactgctgttcccccttgcttctgggcaccactgctgcttcgacccctcagcatggaagttcctggacttcatggatgtttgttccctctgtagcccaagatcctgagttccacaaggtatgtgatgagttcggtttccagtgtcagaatggagtgtgcatcagtttgatttggaagtgcgacgggatggatgattgcggcgattattctgatgaagccaactgcggtaagatgtccagtctgcctcctcacatcctaatcctctagttaacagtgatgcctgcagttacagggacactcaccggcaaccccactgcagacttggcct

SEQ ID NO: 43 - RNA, 350 nt, synthetic construct
  source 1..350: mol_type = other RNA
gtgtgggttgccctggaacttgggtctggaggaggatgcctagagttggagccctgtgttcacactgatgcagggaaagcaactgatgatgtggttgatctctgcagaaaaccccacagaagccccaaactgctcccgctacttccagtttcggtgtgagaatggccactgcatccccaacagatggaaatgtgacagggagaacgactgtggggactggtctgatgagaaggattgtggaggtaagaggcccctggggcctgggttagccccataaccaagacacaccagaatagttccaccagctatgcaggatgtgcctggctgattccgtgttttggggggcatat

SEQ ID NO: 44 - RNA, 370 nt, synthetic construct
  source 1..370: mol_type = other RNA
agcaggtacagctaaaactgggacatccgcactaggtttgggacatatttggtctaagatcaagctgcttctaatgttttgggtttccgtgcttggtgtttttctcagattcacatattcttcccttctcgactcctgggccctccacgtgtctgcccaattactaccgctgcagcagtgggacctgcgtgatggacacctgggtgtgcgacgggtaccgagattgtgcagatggctctgacgaggaagcctgccccttgcttggtgagttctggcccaggtcctctcctgtggtacctgctattgtggagaggaccttctgggggtgtgctctgggcacaatccaaccgggccccatgccctgctgtct

SEQ ID NO: 45 - RNA, 370 nt, synthetic construct
  source 1..370: mol_type = other RNA
tcccattgtaatttctaaagcaccgtaatctcctagcatattgattggttgctgttattggccagctccctcaatattaaaaagtaaattttaaaaatcttttattttagcaaacgtcactgctgcctccactcccacccaacttgggcgatgtgaccgatttgagttcgaatgccaccaaccgaagacgtgtattcccaactggaagcgctgtgacggccaccaagattgccaggatggccgggacgaggccaattgccgtgagtagtcagagagcccttcaccccctgggcacgtttctgcagagagcacagttctggtgcttctgcttcatttctggatcagcacacgcctgtgtgtgagtctgtgt

SEQ ID NO: 46 - RNA, 350 nt, synthetic construct
  source 1..350: mol_type = other RNA
ttgaagcagaagccaattagtactttgcctaaatgggaataaacttgggcccagcctttagtaccatacggcccctccccgtggacttaagaagcctctctgtgtttcagccacacacagcaccttgacttgcatgagcagggagttccagtgcgaggacggggaggcctgcattgtgctctcggagcgctgcgacggcttcctggactgctcggacgagagcgatgaaaaggcctgcagtggtgagtgccggtccacgggctgggctgggctgggctgggctgggaggctcgcttaccccagggcccctcctgtgtagaccttgagctaggacccttttgaactgttgc

SEQ ID NO: 47 - DNA, 20 bp, synthetic construct
  source 1..20: mol_type = other DNA
acactggaagcaatgcctgt

SEQ ID NO: 48 - DNA, 19 bp, synthetic construct
  source 1..19: mol_type = other DNA
cggcactggtgcatttcac

SEQ ID NO: 49 - DNA, 36 bp, synthetic construct
  source 1..36: mol_type = other DNA
agtagcgttcgcccgaacatggcgacacggagcagc

SEQ ID NO: 50 - DNA, 36 bp, synthetic construct
  source 1..36: mol_type = other DNA
ctagtgaggaaagctctttcaggctatcaccatggg

SEQ ID NO: 51 - DNA, 38 bp, synthetic construct
  source 1..38: mol_type = other DNA
ttctagtgaggaaagctctttcaggctatcaccatggg

SEQ ID NO: 52 - DNA, 35 bp, synthetic construct
  source 1..35: mol_type = other DNA
tagtgaggaaagctctttcaggctatcaccatggg

SEQ ID NO: 53 - DNA, 47 bp, synthetic construct
  source 1..47: mol_type = other DNA
ggtcacagatggtggtagcttctttgacacaggtattgttcttgagc

SEQ ID NO: 54 - DNA, 35 bp, synthetic construct
  source 1..35: mol_type = other DNA
tacctgtgtcaaagaagctaccaccatctgtgacc

SEQ ID NO: 55 - DNA, 41 bp, synthetic construct
  source 1..41: mol_type = other DNA
tccggcactggtgcatttggcagtttctctcatcgctcatg

SEQ ID NO: 56 - DNA, 36 bp, synthetic construct
  source 1..36: mol_type = other DNA
cgatgagagaaactgccaaatgcaccagtgccggag

SEQ ID NO: 57 - DNA, 46 bp, synthetic construct
  source 1..46: mol_type = other DNA
cacaggtgtgatagatggcacaatgactttcatcactgttgtctcc

SEQ ID NO: 58 - DNA, 35 bp, synthetic construct
  source 1..35: mol_type = other DNA
tgatgaaagtcattgtgccatctatcacacctgtg

SEQ ID NO: 59 - DNA, 41 bp, synthetic construct
  source 1..41: mol_type = other DNA
atccattgcacttcttctcggtacagttggcttcatcagac

SEQ ID NO: 60 - DNA, 37 bp, synthetic construct
  source 1..37: mol_type = other DNA
tgaagccaactgtaccgagaagaagtgcaatggattc

SEQ ID NO: 61 - DNA, 41 bp, synthetic construct
  source 1..41: mol_type = other DNA
agtgcgtacagaggggctcacagttgactggatcctcatcg

SEQ ID NO: 62 - DNA, 37 bp, synthetic construct
  source 1..37: mol_type = other DNA
ggatccagtcaactgtgagcccctctgtacgcacttc

SEQ ID NO: 63 - DNA, 36 bp, synthetic construct
  source 1..36: mol_type = other DNA
ggaactcaggatcttgggcgcagtgctgttcatcgg

SEQ ID NO: 64 - DNA, 35 bp, synthetic construct
  source 1..35: mol_type = other DNA
cgatgaacagcactgcgcccaagatcctgagttcc

SEQ ID NO: 65 - DNA, 38 bp, synthetic construct
  source 1..38: mol_type = other DNA
gaagggaagaatatgtgaatagcatcctgcaaacgccg

SEQ ID NO: 66 - DNA, 42 bp, synthetic construct
  source 1..42: mol_type = other DNA
tgcggcgtttgcaggatgctattcacatattcttcccttctc

SEQ ID NO: 67 - DNA, 44 bp, synthetic construct
  source 1..44: mol_type = other DNA
agggaagaatatgtgaatcgcagttggcttcatcagaataatcg

SEQ ID NO: 68 - DNA, 39 bp, synthetic construct
  source 1..39: mol_type = other DNA
tgatgaagccaactgcgattcacatattcttcccttctc

SEQ ID NO: 69 - DNA, 42 bp, synthetic construct
  source 1..42: mol_type = other DNA
aggcagcagtgacgtttgctccacaatccttctcatcagacc

SEQ ID NO: 70 - DNA, 36 bp, synthetic construct
  source 1..36: mol_type = other DNA
tgagaaggattgtggagcaaacgtcactgctgcctc

SEQ ID NO: 71 - DNA, 37 bp, synthetic construct
  source 1..37: mol_type = other DNA
tcaaggtgctgtgtgtggcaagcaaggggcaggcttc

SEQ ID NO: 72 - DNA, 36 bp, synthetic construct
  source 1..36: mol_type = other DNA
agcctgccccttgcttgccacacacagcaccttgac

SEQ ID NO: 73 - DNA, 36 bp, synthetic construct
  source 1..36: mol_type = other DNA
tgtacacagtcaactcatggcaattggcctcgtccc

SEQ ID NO: 74 - DNA, 38 bp, synthetic construct
  source 1..38: mol_type = other DNA
ggacgaggccaattgccatgagttgactgtgtacaaag

SEQ ID NO: 75 - DNA, 38 bp, synthetic construct
  source 1..38: mol_type = other DNA
gcagtagcgttcgcccgaacatggcgacacggagcagc

SEQ ID NO: 76 - DNA, 50 bp, synthetic construct
  source 1..50: mol_type = other DNA
tcccctgccgcactctgatgggtagagaacacctgtcttcgcaaccagta

SEQ ID NO: 77 - DNA, 50 bp, synthetic construct
  source 1..50: mol_type = other DNA
gacatgagcgatgagagaaactgccgtgagtcttctggattggacgttaa

SEQ ID NO: 78 - DNA, 50 bp, synthetic construct
  source 1..50: mol_type = other DNA
atgttgtctttctatcccattttagctaccaccatctgtgacctggacac

SEQ ID NO: 79 - DNA, 50 bp, synthetic construct
  source 1..50: mol_type = other DNA
gacaacagtgatgaaagtcattgtggtaaggggacatcacatcctgaaac

SEQ ID NO: 80 - DNA, 50 bp, synthetic construct
  source 1..50: mol_type = other DNA
tctctgtttatggtctcacctgcagaaatgcaccagtgccggagtgacga

SEQ ID NO: 81 - DNA, 50 bp, synthetic construct
  source 1..50: mol_type = other DNA
tggtctgatgaagccaactgtaccggtcagtacttcctggactcagttga

SEQ ID NO: 82 - DNA, 50 bp, synthetic construct
  source 1..50: mol_type = other DNA
actgtgtctctcttctctgttacagccatctatcacacctgtgaggcctc

SEQ ID NO: 83 - DNA, 50 bp, synthetic construct
  source 1..50: mol_type = other DNA
tccgatgaggatccagtcaactgtggtaaatgcaaattccccagctccct

SEQ ID NO: 84 - DNA, 50 bp, synthetic construct
  source 1..50: mol_type = other DNA
tctgtgttgttgaattctatttcagagaagaagtgcaatggattccgctg

SEQ ID NO: 85 - DNA, 50 bp, synthetic construct
  source 1..50: mol_type = other DNA
gatggctccgatgaacagcactgcggtgagttcattccttgcccccagga

SEQ ID NO: 86 - DNA, 50 bp, synthetic construct
  source 1..50: mol_type = other DNA
cttcccttctctggatcccttacagagcccctctgtacgcacttcatgga

SEQ ID NO: 87 - DNA, 50 bp, synthetic construct
  source 1..50: mol_type = other DNA
gaggatgcggcgtttgcaggatgctgtgagttggggcaggcaggggaggt

SEQ ID NO: 88 - DNA, 50 bp, synthetic construct
  source 1..50: mol_type = other DNA
tcatggatgtttgttccctctgtagcccaagatcctgagttccacaaggt

SEQ ID NO: 89 - DNA, 50 bp, synthetic construct
  source 1..50: mol_type = other DNA
gattattctgatgaagccaactgcggtaagatgtccagtctgcctcctca

SEQ ID NO: 90 - DNA, 50 bp, synthetic construct
  source 1..50: mol_type = other DNA
ctgatgatgtggttgatctctgcagaaaaccccacagaagccccaaactg

SEQ ID NO: 91 - DNA, 50 bp, synthetic construct
  source 1..50: mol_type = other DNA
tggtctgatgagaaggattgtggaggtaagaggcccctggggcctgggtt

SEQ ID NO: 92 - DNA, 50 bp, synthetic construct
  source 1..50: mol_type = other DNA
tttccgtgcttggtgtttttctcagattcacatattcttcccttctcgac

SEQ ID NO: 93 - DNA, 50 bp, synthetic construct
  source 1..50: mol_type = other DNA
gacgaggaagcctgccccttgcttggtgagttctggcccaggtcctctcc

SEQ ID NO: 94 - DNA, 50 bp, synthetic construct
  source 1..50: mol_type = other DNA
aaattttaaaaatcttttattttagcaaacgtcactgctgcctccactcc

SEQ ID NO: 95 - DNA, 50 bp, synthetic construct
  source 1..50: mol_type = other DNA
gatggccgggacgaggccaattgccgtgagtagtcagagagcccttcacc

SEQ ID NO: 96 - DNA, 50 bp, synthetic construct
  source 1..50: mol_type = other DNA
cttaagaagcctctctgtgtttcagccacacacagcaccttgacttgcat

SEQ ID NO: 97 - DNA, 50 bp, synthetic construct
  source 1..50: mol_type = other DNA
gagagcgatgaaaaggcctgcagtggtgagtgccggtccacgggctgggc